(19) United States
(12) Patent Application Publication
(10) Pub. No.: US 2021/0254021 A1
(43) Pub. Date: Aug. 19, 2021

(54) INTEGRATED MANUFACTURING AND CHROMATOGRAPHIC SYSTEM FOR VIRUS PRODUCTION

(71) Applicant: THEMIS BIOSCIENCE GMBH, VIENNA (AT)

(72) Inventors: Patrick CSAR, KÖNIGSTETTEN (AT); Alois JUNGBAUER, VIENNA (AT); Alexander KORT, VIENNA (AT); Matthias MÜLLNER, PIXENDORF (AT); Dieter PALMBERGER, VIENNA (AT); Katrin RAMSAUER, VIENNA (AT); Sabrina SCHRAUF, MÖRBISCH (AT); Petra STEPPERT, SCHWADORF (AT); Erich TAUBER, MUCKENDORF (AT); David A. MAIER, VIENNA (AT); Jochen STRITZKER, KIERLING (AT)

(21) Appl. No.: 17/252,210

(22) PCT Filed: Jun. 14, 2019
(86) PCT No.: PCT/EP2019/065670

§ 371 Date: Dec. 14, 2020

(30) Foreign Application Priority Data
Jun. 15, 2018 (EP) 18178037.0

(51) Int. Cl. C12N 7/00; C12M 1/00; B01D 61/14; B01D 15/34; B01D 15/32; B01D 15/36

(52) U.S. Cl. CPC C12N 7/00; C12M 43/00; C12M 47/02; B01D 61/145; B01D 15/34; B01D 2311/2626; B01D 15/361; C12N 2760/18423; C12N 2760/18451; B01D 2315/10; B01D 2315/08; B01D 15/327

(57) ABSTRACT

Provided is a method for producing and/or purifying measles virus (MV) particles from a sample, the method comprising in sequential order the following steps loading a sample containing MV particles and one or more impurities onto a stationary phase material for carrying out flow-through chromatography to bind at least a fraction of the impurities contained in the sample and to produce a flow-through comprising at least a fraction of the MV particles contained in the sample; carrying out filtration, preferably ultrafiltration, and obtaining a retentate having an increased MV titer relative to the MV titer comprised in the flow-through. Further provided is a system for producing and/or purifying MV particles, comprising at least one bioreactor; a clarification unit, preferably a dead end filter unit, downstream to the bioreactor; a flow through chromatography unit downstream to the clarification unit; and a filtration unit, downstream to the flow through chromatography unit.

### TECHNICAL FIELD

The present invention generally relates to the field of virology and specifically relates to chromatography based purification strategies of sterically demanding, i.e. large and pleomorphic, virus particles, particularly measles viruses (MVs) and more particularly viruses having a MV scaffold to yield fractions or compositions comprising virus particles in high yield and low content of impurities such as host cell DNA contaminants and/or host cell protein contaminants.

### BACKGROUND OF THE INVENTION

Virus particles are used in a variety of different prophylactic and curative medical applications, e.g., for vaccination as well as for therapeutic purposes. Regulatory agencies demand the provision of specifically produced virus material to guarantee the safety of the respective material. The WHO and the responsible national and regional approval authorities, like the FDA or the EMEA, understandably impose high product and labelling requirements to a composition comprising a virus material used as vaccine or therapeutic to achieve the provision of safe biological products. The major hallmarks to be fulfilled by a vaccine candidate based on a virus or viral component are its safety, purity and potency. Therefore, there exists an ongoing need to provide suitable vaccines having an acceptable immunogenicity, safety and tolerability profile for vaccination.

Virus particles find also application in oncolytic virus (OV) therapy, which has been recognized as a promising and potentially break-through therapeutic approach for cancer treatment. OVs are genetically engineered or naturally occurring viruses that can selectively replicate in and kill cancer cells without, or at least to a less extent, harming benign tissues. In contrast to gene therapy where a virus is used as a mere carrier for transgene delivery, OV therapy uses the virus itself as an active ingredient. OVs implement a unique mode of action, tumor-restricted viral infection, replication, cell lysis and spread. Preclinical and clinical research has revealed their pleiotropic therapeutic activity: (i) viral tumor cell lysis triggers systemic antitumor immunity, (ii) the insertion of therapeutic genes can trigger bystander killing by different means, depending on the chosen gene, and (iii) endothelial cells specifically in tumor vessels are susceptible to OVs, resulting in vascular shut down and indirect destruction of tumor cells. Whereas numerous oncolytic viruses have been subjected to clinical trials, the common feature that is expected to play a major role in prolonging the survival of cancer patients is an induction of specific antitumor immunity in the course of tumor-specific viral replication (for a review, see Fukuhara et al., Cancer Sci. 2016 October; 107(10): 1373-1379).

In contrast to using virus particles as a vaccine, oncolytic activity as an advanced therapy medicinal product depends on high concentration of infectious particles. Product- and process-related impurities are an inherent problem when working with viruses due to the fact that a virus will have to be propagated on suitable host cell in a suitable manner. Though batch preparations meet the requirements for clinical trials, purity needs to be improved to bring an OV therapeutic product to the market. The ratio between total virus particles (vp) and infectious particles (ip) is a critical parameter for the quality of virus preparations. Although a certain vp/ip ratio is not specified by the regulatory agencies, a value of ≈50 in virus based preparations can be assumed on average. Residual DNA from host cells in the final product is one of the concerns in the manufacturing process of MV as an advanced therapy medicinal product. For viral vaccines, the standard limits adopted by various countries (U.S. Pharmacopoeia, European Pharmacopoeia and Pharmacopoeia of the People's Republic of China) for viral vaccines are mainly three grades: 10 ng/dose, 100 pg/dose and 10 pg/dose. But for the use of high doses of MV in cancer therapies (e.g., 10⁹ ip for intratumoral injections), the limit for expectedly higher amounts of residual host DNA in the final product still needs to be established (Mol. Ther. Methods Clin. Dev. 2016; 3: 16018). Thus, there is a need for providing a method for the provision of virus based preparations suitable for OV therapy, which method allows the production of virus particles in high yield and high purity, and which can be carried out aseptically under GMP conditions throughout the whole process.

Viruses are a highly heterogeneous group of infectious agents. An enormous variety of possible genomic compositions for different virus groups can be observed. The major virus groups are double-stranded (ds) DNA viruses, single-stranded (ss) DNA viruses, dsRNA viruses, (+strand) ssRNA viruses, (-strand) RNA viruses, ssRNA retroviruses and dsDNA retroviruses. In turn, viruses display a wide diversity of shapes and sizes, also called morphologies. This diversity strongly influences purification strategies, which have to be optimized individually. A complete virus particle or virion usually consists of nucleic acid (RNA or DNA) surrounded by a protective coat of protein called a capsid. Capsids are formed from identical protein subunits called capsomeres. Certain viruses can have a lipid “envelope” derived from the host cell membrane. The capsid comprises proteins encoded by the viral genome and its shape serves as the basis for morphological distinction. Viruses in the size range of between about 20 and 800 nm have been described, whereas some filoviruses may have a total length of up to 1,400 nm. A particular problem for purification approaches may be pleomorphic viruses, i.e., the appearance and shape of one and the same virus can vary within a given preparation.

Based on the nature of the different groups of viruses, the amount of residual cell-substrate DNA in a viral preparation, e.g., a vaccine, will depend on the degree to which the vaccine can be purified. For example, a protein vaccine or a subunit vaccine, such as an influenza vaccine, would usually have less contaminating DNA than an inactivated, whole virus vaccine, such as the inactivated poliovirus vaccine, and both of these would have less DNA than a live attenuated vaccine, such as the OPV (oral polio vaccine), MMR (measles—mumps—rubella), or varicella vaccines.

With regard to purification of virus particles, Nestola et al. (Biotechnology and Bioengineering, Vol. 112, No. 5, May, 2015) points out that viruses and viral vectors are complex biopharmaceutical products, which vary in size, shape, and surface structure. The isoelectric point (pi), surface hydrophobicity, presence or absence of an envelope, and particle lability can play important roles in the design of the DSP train. The virus surface defines its individual physicochemical characteristics including the charge magnitude and distribution, hydrophobic residues, and post-translational modifications (i.e., glycans) of surface proteins. Compared to classic monoclonal antibodies, further additional aspects have to be taken into consideration in virus production: the bioreaction and purification steps must maintain the immunogenicity and stability of the viruses or viral vectors throughout the entire production chain. Moreover, safety usually, biosafety level 2 or higher is required. In addition, the complexity of viruses requires refined analytical techniques for assessing the purity and quality attributes of the final product. Given this complexity, an effective and scalable purification process of the bioproduct can only be achieved through a targeted and fine-tuned combination of several unit operations which necessarily have to be modified for each different virus or virus particle.

Ultrafiltration (UF) is considered a key operation in all large-scale bioprocesses that produce large volumes of bulk, e.g., up to 2 kL for vaccines and 20 kL for mAbs, since they must be concentrated 10-100 times prior to being further purified by chromatography. The volumetric concentration and optional buffer exchange of the virus bulk is critical not only to obtain high titer stocks in the proper formulation buffer, but also to reduce the handled volume; the latter accelerates the downstream processing and keeps the scalability of the purification train at a manageable level (Nestola et al., supra).

One specific virus which is known to be difficult to purify is measles virus (MV), said virus being inherently pleomorphic. MV shows polymorphisms in both morphology and viral particle size making this virus particularly hard to purify in comparison to other viruses showing a more homogeneous size distribution and having a much smaller overall particle size. For example, Japanese encephalitis virus (JEV), a flavivirus, was shown to have a virus particle size for only 40 to 50 nm in size in infected Vero cells (Yang et al., J. Vet. Sci., (2004); 5(2):125-130, “Biophysical characterization of Japanese encephalitis virus (KV1899) isolated from pigs in Korea”). In contrast, MV particles were shown to have particle size distributions on the range from 50 to 1,000 nm with a major distribution peak with diameters of 350 to around 400 nm (Daikoku et al., Bulletin of the Osaka Medical College, 53(2):107-114, 2007, “Analysis of Morphology and Infectivity of Measles Virus Particles”). Particularly large MV particles possess the MV M and H proteins and a nucleocapsid are thus considered as “complete” virus particles. In turn, in particular these large particles are infectious, a prerequisite for designing functional vaccines or OV therapies based on modified MVs. Likewise, these large particles are inherently hard to purify in comparison to other viruses in a functional way, i.e., as infections MV particles, and simultaneously with acceptable yields.

WO 2016/156613 A1 suggests a process for MV purification. This process integrates a batch adsorption process on a chromatography resin, i.e., the material is adsorbed but fails to provide a fully integrated flow-through chromatography technique for MV purification. As such, the disclosed methods suffer from the problems associated with batch adsorption purification that not all the adsorbent in the column may be used efficiently. Further, quite large amounts of eluent are needed to elute the separated components, resulting in dilution of the products. Moreover, highly purified products may not be obtained whenever the differences in adsorption affinities of the two components for the adsorbent (i.e., selectivity) are small. Finally, batch adsorption represents a discontinuous process. These facts make batch adsorption unsuitable for large scale purifications of MV particles (i) under GMP conditions for providing (ii) high yields of a (iii) fully functional and infectious MV product.

FR 3 014 901 A1 discloses methods for purifying pseudotyped retroviruses or lentiviruses, but fails to disclose a purification strategy for an infectious MV particle. By their very nature, retroviruses are generally spherical enveloped particles with an average diameter ranging between around 100 to 200 nm only. A retrovirus may be pseudotyped which implies that foreign viral envelope proteins or glycoproteins are introduced, for example, into a retroviral or lentiviral vector. The resulting pseudotyped virus particle still has the major characteristics of the “original” virus and additionally carries the inserted elements of the foreign virus.

Despite the availability of first vaccine candidates based on MV particles, there exists an ongoing need to provide purification schemes for removing product-related impurities from virus preparations intended for therapy, particularly if derived from sterically demanding large viruses, for example, from the order Mononegavirales, particularly the family Paramyxoviridae, for example MV particles, simultaneously allowing the provision of an infectious and immunogenic virus population, which can be conducted under GMP conditions. A reasonable and preferably continuous flow-through chromatography purification scheme for MV would thus be needed to overcome the disadvantages of present MV purification strategies. Moreover, there exists an ongoing need to provide purification schemes for virus particles in high yields, in particular when the virus particles are intended to be used in OV therapy, which demands for much higher doses than vaccination. Moreover, there exists an ongoing need to provide virus particles in high purity to allow the provision of a safer medicament finding more acceptance and being faced with less concerns due to their impurities, in particular in case it has to be administered in high doses such as in OV therapy.

### SUMMARY OF THE INVENTION

An object of the present invention is thus the provision of a method suitable for the purification of MVs, in particular recombinant MV but also applicable to wild-type structures, which allows the provision of a highly pure viral composition with high yields. Another object of the present invention was to establish a method suitable for the purification of MVs, which method can be carried out as a fully-integrated process. The process should be a closed process having end-to-end functionality to comply with sterility requirements throughout the whole process. Furthermore, it was an aim to establish a method suitable for the purification of MVs, which method can be precisely controlled.

These objects have been achieved by providing, in a first aspect, a method suitable for purifying MV particles from a sample, the method comprising in sequential order the following steps: loading a sample containing MV particles and one or more impurities onto a stationary phase material and carrying out flow-through chromatography to bind at least a fraction of the one or more impurities contained in the sample and to produce a flow-through comprising at least a fraction of the MV particles contained in the sample; and carrying out filtration, preferably ultrafiltration, and obtaining a retentate having an increased MV titer relative to the MV titer comprised in the flow-through.

The objects have been further achieved by providing in a second aspect a system suitable for purifying MV particles, the system comprising or consisting of: (i) at least one bioreactor; (ii) a clarification unit, preferably a dead end filter unit, downstream to the bioreactor; (iii) a flow through chromatography unit downstream to the clarification unit; and (iv) a filtration unit, preferably a tangential flow filtration unit, downstream to the flow through chromatography unit.

In a third aspect of the disclosure, the objects have been achieved by use of a flow-through chromatography unit as defined in the second aspect of the present invention for the purification of MV particles.

The first and second aspect of the present invention are particularly useful in providing a prophylactic and/or therapeutic composition. Hence, in a fourth aspect of the present disclosure, there is provided a prophylactic or therapeutic composition produced by the method of the first aspect of the invention or the system of the second aspect of the invention, wherein infectious MV particles are comprised as an active ingredient.

Preferred prophylactic and/or therapeutic compositions include immunogenic and/or vaccine compositions, which are suitable and/or intended to be used in a method of eliciting an immune response or in a method of prophylactic treatment of a subject for protecting the subject from infection with a virus. In such method, protection is achieved by exposing the subject to the MV particles comprised by the immunogenic composition or the vaccine composition. Further preferred prophylactic and/or therapeutic compositions include compositions, which are suitable and/or intended to be used for tumor therapy such as oncolytic tumor therapy based on infectious MV particles.

Hence, in a fifth aspect of the present disclosure, there is provided a composition according to the fourth aspect of the present invention for use in a method of therapeutic and/or prophylactic treatment. In one embodiment of the fifth aspect, a subject is exposed to the MV particles comprised by the composition for protecting the subject from infection with a virus and/or an immune response is elicited by exposing the subject to the MV particles comprised in the composition. In another embodiment of this aspect, a subject is exposed to the MV particles comprised by the composition for tumor therapy such as oncolytic tumor therapy and/or an immune response is elicited by exposing the subject to the MV particles comprised in the composition.

In a sixth aspect of the present disclosure, there is provided a method of prophylactic and/or therapeutic treatment, the method comprising exposing a subject to the MV particles comprised by the composition of the fourth aspect of the present disclosure. In one embodiment of this aspect, the method is used for protecting the subject from infection with a virus. In another embodiment of this aspect, the method is used for (oncolytic) tumor therapy. In both cases, the method may comprise exposing the subject to the MV particles comprised in the composition and/or elicit an immune response by exposing the subject to the MV particles comprised in the composition.

In a seventh aspect, the present disclosure relates to use of MV particles, obtained by the method according to the first aspect or the system according to the second aspect of the invention, for the manufacture of a medicament for therapeutic and/or prophylactic treatment, in particular as a vaccine or in OV therapy.

As the various embodiments and aspects encompassed by the present disclosure all relate to possible variations of the methods of the present invention, they can be used alone or in combination with each other all forming individual embodiments according to the various aspects according to the present disclosure, where reasonable for the skilled person having knowledge of the present disclosure. Especially, all embodiments disclosed for the first aspect according to the present invention likewise apply for the second aspect of the present application, as the system of the invention is suited and intended to be used for carrying out the various embodiments of the method according to the first aspect of the invention.

Further aspects and embodiments of the present invention can be derived from the subsequent detailed description and drawings, the sequence listing, as well as the attached set of claims.

### BRIEF DESCRIPTION OF THE DRAWINGS

FIG. 1 show a process scheme of upstream manufacturing in accordance with an embodiment of the first aspect of the present invention. Arrows indicate: (A) inoculation of bioreactor; (B) infection of bioreactor; (C) harvest.

FIG. 2 shows a process scheme of downstream manufacturing in accordance with a further embodiment of the first aspect of the present invention. Arrows indicate: (A) A: 1 M MgCl2, 500 mM EDTA; B: A: 50 mM HEPES, 150 mM NaCl, pH 7.5, (B) 50 mM HEPES, 2M NaCl, pH 7.5; (C) 50 mM HEPES, 150 mM NaCl, pH 7.5; (D) 5% Sorbit (w/v); (E) 1 M NaOH, 0.1 M NaOH, WFI, 20% EtOH; (F) 1 M NaOH, WFI, 50 mM HEPES, 150 mM NaCl, pH 7.5; (G) 1 M NaOh, 0.1 M NaOH, WFI, 20% EtOH.

FIG. 3 shows attachment to and growth of host cells on microcarriers, which can be employed in certain embodiments of the first aspect of the present invention. In this specific embodiment, Vero cells and Cytodex I were used as host cells and microcarriers, respectively. FIG. 3A is a photograph taken 1 h after inoculation. As outlined above, “inoculation” means combining host cells, or a host cells containing liquid, and microcarriers, or a microcarrier containing liquid, in a common mixture under condition which allows the host cells to adhere to the microcarriers. As can be observed, the host cells readily attached to the microcarriers. FIG. 3B is a photograph taken 96 h after inoculation and demonstrates that the cells grow until confluence. At or around this point, the host cells are ready for virus infection, which time point will vary depending on the nature of the host cell.

FIG. 4 show cell growth and metabolite consumption over time following inoculation. FIG. 4A shows development of cell numbers. The solid line indicates counts of attached cells, whereas the dotted line indicates counts of suspended cells. As can be seen, upon attachment, cells immediately start to grow until confluence resulting in a cell density of up to 1.5×10⁶ cells/mL 96 h post inoculation. After a medium exchange, the culture was infected at 96 h post inoculation resulting in a growth arrest and finally leading to cell detachment (decrease of solid line; increase of dashed line) form the microcarrier and cell lysis. FIG. 4B shows the course of various metabolites following inoculation. During a first growth phase lasting until 96 h post infection, cells metabolize nutrients provided by the medium thereby producing toxic by-products. In accordance with this, glucose (dashed line) and glutamine (dotted line) were consumed in relation to cell growth, whereas lactate (solid black line) and ammonium (solid grey line) were build up. In order to improve effectiveness of infection with and production of MV, the medium was renewed after at 96 h post inoculation.

FIG. 5 shows the time-dependent increase of virus titer expressed by means of TCID₅₀ (median tissue culture infective/infectious dose) post infection. As a result of successful virus amplification, the virus titer steadily increases. A top level of 9.55×10⁵ TCID₅₀/mL is achieved at 144 h post infection. The bar on the right hand (“filt. Harvest”) illustrates the virus titer determined after clarification using depth filtration. By comparing it with the respective virus titer at 144 h post infection, one could deduce that clarification results in an increase of the virus titer. This observation could be explained in that clarification and in particular filtration might lead to a break up of virus aggregates, thereby increasing the number of infectious virus particles.

FIG. 6 shows on-line cell density measurements in accordance with an embodiment of the first aspect of the present invention. On-line signals were generated by permittivity measurements of live cells (solid line). Comparative off-line data were generated by measurement of cellular nuclei after citric acid release (dashed line). As can be seen, the on-line signal immediately reacts on the inoculation of the bioreactor and continuously increases upon confluence of the cells on the microcarrier and depletion of the medium. Off-line values are in good agreement with on-line counterparts and deviations can be explained by technical issues during off-line measurements. 96 h post inoculation the medium is exchanged, and the culture is infected with MV. This causes a stop in cell growth followed by a detachment and lysis of the cells.

FIG. 7 shows data generated during two individual purification runs using flow-through chromatography in 1 mL scale in accordance with an embodiment of the first aspect of the present invention. In a first experiment, 35 CV (column volume(s)) of filtered cell culture supernatant was directly loaded on a 0.844 mL Capto Core 700 column. In a second experiment, 44 CV of filtered and endonuclease treated cell culture supernatant was directly loaded on a 0.903 mL Capto Core 700 column. Results of the first experiment are shown in FIGS. 7A to 7D. Results of the second experiment are shown in FIGS. 7E and 7G. More specifically, FIGS. 7A and 7E show chromatograms, FIGS. 7B and 7F show Western blot analyses of elution fractions using Measles NP (3E1) antibody, FIG. 7C show a Coomassie-stained gel and FIGS. 7D and 7G show a silver-stained gel. The analyzed elution fractions are indicated in the respective chromatograms. The fractions are abbreviated as follows. L: Loading material; BH: Bulk harvest; FT: Flow-through; W: Wash; R: Regenerate. The Western blot analyses also show a sample, which was desorbed from the frit of the Tricorn column as outlined herein. This sample is indicated as F: Frit Tricorn column. It was thereby proven than the MV adsorbed to the frit, resulting in a low recovery.

FIG. 8 shows on-line data (FIGS. 8A and 8E), Western blot analysis (FIGS. 8B and 8E) as well as silver-stained (FIGS. 8D and 8H) and Coomassie-stained SDS-PAGE gels (FIGS. 8C and 8G), which were generated during further flow-through chromatography runs, upscaled to 10.5 mL (FIGS. 8A to 8D) or 188.5 mL (FIGS. 8E to 8H) Capto Core 700 column, in accordance with an embodiment of the first aspect of the present invention. Elution fractions were collected according to the chromatogram and analyzed by Western blot detecting Measles NP (3E1). The following abbreviations were used for denoting the fractions. L: Loading material; BH: Bulk harvest; FT: Flow-through; W: Wash; R: Regenerate; FH: Filtered harvest.

FIG. 9 shows ultrafiltration of MVs material that has been purified by flow-through chromatography in accordance with an embodiment of the first aspect of the present invention. The purified MVs material was concentrated by a HF module (FIGS. 9A to 9D), a RC membrane (FIGS. 9E to 9H) or SC membrane (FIGS. 9I to 9L). All membranes providing 50 cm² filtration area and 300 kDa MWCO (FIGS. 9A to 9H) or 30 kDa MWCO (FIGS. 9I to 9L). The moving average of the permeate flux Samples of the feed (indicated with letter F), the concentrate (indicated with letters C, C1, C2), and permeate (indicated with letter P) were collected during the filtration according to the labels in diagrams (FIGS. 9A, 9E and 9I) and were analyzed by Western blot detecting Measles NP (3E1) (FIGS. 9B, 9F, 9J) and SDS-PAGE analysis Coomassie-stained (FIGS. 9C, 9G, 9K) or silver stained (FIGS. 9D, 9H, 9L).

FIG. 10 shows a process scheme according to an embodiment of the method of the present invention. Recoveries (yields) in terms of TCID50/mL as well as relative recoveries calculated from TCID50/mL are indicated for each step. Furthermore, relative amounts of impurities, specifically total protein content and dsDNA content, are indicated. For example, according to this embodiment, the optional endonuclease treatment step results in an about 60% reduction in the amount of dsDNA. Notably, the depletion factors are determined after they have been correlated to the respective volume, i.e., the total amount (conc.*volume) is 100% of virus, protein or DNA, which have been used for a specific step. The % values after each step thus indicate the amount (conc.*volume) which could be recovered.

FIG. 11 shows the results of viscosity measurement of process fluid, measured after each process step.

FIG. 12 shows results obtained from chromatographic purification of GFP-MV by Capto Core 700 in accordance with an embodiment of the first aspect of the present invention. The chromatogram (FIG. 12A) shows inter alia an online GFP signal measured at Absorbance of 490 nm. Western blot analysis (FIG. 12B) were carried out by detecting Measles NP (3E1). L: Loading material; BH: Bulk harvest; FT: Flow-through.

FIG. 13 shows fluorescence microscopic pictures taken after infection of Vero host cells with MV-GFP in accordance with an embodiment of the first aspect of the present invention.

FIG. 13A: 24 h post infection; FIG. 13B: 48 h post infection; FIG. 13C: 72 h post infection; FIG. 13D: 96 h post infection; FIG. 13E: 120 h post infection; and FIG. 13F: 144 h post infection. The largest image (FIGS. 13A to F) always shows the increasing GFP expression (white/bright spots in black/and white drawings). The large image on the left is a merge of the upper image on the right and the lower image on the right. The lower image on the right shows the GFP-fluorescence channel (again, white/bright spots indicated GFP signals), whereas the upper image on the right shows the bright field image. As can be seen, the number of GFP expressing cells (bright spots in the respective image) on the microcarrier increased over time indicating success of infection and production of MV-GFP. With increasing duration of incubation following infection, spots were also visible in the culture broth as a result of cell lysis and release of MV.

### DEFINITIONS

When reference is made to “a” or “an” entity this means that one, or more than one, of that entity is meant, unless otherwise dictated by context. For example, “a nucleotide sequence” is understood to represent a first embodiment, wherein one nucleotide sequence is meant, and a second embodiment, wherein more than one nucleotide sequences are meant. As such, the terms “a” (or “an”), “one or more” and “at least one” can be used interchangeably herein.

The expression “and/or” as used herein shall be understood as specific disclosure of each of the two specified features or components with or without the other. Thus, the term “and/or” as used in a phrase such as “A and/or B” herein includes “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the respective numerical values by a variance. When the term “about” is used in conjunction with a numerical value, it modifies that value above and below the stated value by a variance. In the context of the present invention, a variance is to be taken to include maximum values of 50%, 40%, 30%, 20% or 10%.

An “adjuvant” in the field of immunology and as used herein refers to a substance or composition enhancing antigenicity of another substance. This is achieved by specifically stimulating and balancing mainly the Th1 and Th2 subsets of T-cells and thus their effector molecules. Usually, immunogenic compositions based on live-attenuated or killed viruses are highly immunogenic per se and thus there might not be the need for an additional adjuvant, whereas an additional adjuvant might still be favorable to balance the provoked immune response. Th1 cells secrete IFN-γ, which activates macrophages and thus induces the production of opsonizing antibodies by B cells. The Th1 response therefore leads mainly to a cell-mediated immunity. Th2 cells mainly secrete cytokines, including IL-4, which induces B cells to make neutralizing antibodies. Th2 cells generally induce a humoral (antibody) response critical in the defense against extracellular pathogens (helminths, extracellular microbes and toxins).

The terms “amino acid molecule/sequence”, “protein”, or “peptide” or “polypeptide” are used interchangeably herein. The term “amino acid” or “amino acid sequence” or “amino acid molecule” comprises any natural or chemically synthesized protein, peptide, or polypeptide or a modified protein, peptide, polypeptide and enzyme, wherein the term “modified” comprises any chemical or enzymatic modification of the protein, peptide, polypeptide and enzyme.

The term “antigen” as used herein and as commonly used in the field of immunology refers to an “antibody generating” molecule, i.e. a substance, which can elicit an adaptive immune response. An antigen is thus a molecule binding to an antigen-specific receptor, either a T-cell or a B-cell receptor. An antigen is usually a (poly)peptide, but it can also be a polysaccharide or a lipid, possibly combined with a protein or polysaccharide carrier molecule. For the purpose of the various aspects and embodiments of the present invention, the antigen is a (poly)peptide, i.e. an amino acid sequence. In the case of binding to a T-cell receptor, the antigen is presented to the respective T-cell receptor via an antigen-presenting cell as an antigenic peptide bound to a histocompatibility molecule on the surface of the antigen presenting cell, wherein the antigenic peptide has been processed in advance by the antigen presenting cell. Thus, an “antigen” as used herein refers to a molecule, such as a protein or a polypeptide, containing one or more epitopes that will stimulate a host's immune system to make a humoral and/or cellular antigen-specific response. The term is also used interchangeably with the term “immunogen” and concerning the effect “immunogenic”.

As used herein, the term “aseptic” denotes condition, under which contamination from living organisms prevented. In the context of the present invention, an aseptic operation results in a product having low bioburden or even being substantially sterile.

The term “at least a fraction” in the context of binding impurities includes embodiments, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or even 100% of the one or more impurities bind to a chromatography material and is thereby separated from the product of interest.

The term “at least one host cell” may generally refer to one host cell, more than one host cell and preferably a plurality of host cells (herein sometimes also denoted a “cell population”). The term host cell encompasses non-recombinant cells, i.e. cells that were not immortalized or transformed or manipulated in a purposive manner, as well as recombinant host cells or immortalized host cells (spontaneous or immortalized in a targeted manner). To be suitable for the purposes of the present invention, the host cell(s) must be able to support the MV replication cycle, i.e. the cell(s) must be susceptible to MV or MV scaffold infection and suitable for the subsequent propagation or replication cycle, including replication, translation, encapsidation of the RNA of the virus and budding from the host cell to be released as virus particle. Preferred eukaryotic host cells that fulfil these requirements are disclosed herein.

The terms “attenuation” or “attenuated” as used herein in connection with a virus or a material derived therefrom refers to a virus weakened under laboratory conditions which is less vigorous than the respective wild-type virus, for example by deleting certain genes, for example a viral accessory protein of the virus. An attenuated virus may be used to make a vaccine that is capable of stimulating an immune response and creating immunity.

“Binding” a molecule such as an impurity to a chromatography material means exposing the molecule to chromatography material under appropriate conditions (e.g. pH/conductivity) such that the molecule is reversibly retained and/or immobilized in or on the chromatography resin by virtue of ligand-molecule interactions.

The term “cDNA” stands for a complementary DNA and refers to a nucleic acid sequence/molecule obtained by reverse transcription from an RNA molecule.

The term “clarifying” according to the present disclosure refers to a step for removing large impurities from a bulk product such as a cell culture supernatant to be clarified. The term removing also encompasses a reduction in the amount (e.g. concentration) of impurities. Clarification may rely on separation by size, molecular weight or density and the like. “Large impurities” is to be taken to preferably refer to a size bigger than the MV particles to be purified.

A “carrier” according to the present disclosure is a substance that aims at improving the delivery and effectiveness of a drug composition. Carrier materials may depend on the physical state of a drug composition to be administered. Typically, immunogenic or vaccine compositions are administered as liquid solution. Suitable substances include, large, slowly metabolized macromolecules, such as proteins, polysaccharides, polylactic acids, polyglycolic acids, polymeric amino acids, amino acid copolymers, and inactive virus particles. Pharmaceutically and veterinary acceptable salts can also be used in the immunological composition, for example, mineral salts such as hydrochlorides, hydrobromides, phosphates, or sulfates, as well as the salts of organic acids such as acetates, propionates, malonates, or benzoates. Immunological compositions can also contain liquids, such as water, saline, glycerol, and ethanol, as well as substances such as wetting agents, emulsifying agents, or pH buffering agents. Furthermore, nanocarriers, including liposomes, also can be used as carriers. Depending on the nature of the therapeutic or prophylactic composition used and dependent on the immune response, which is intended to be provoked, such a composition can additionally comprise an adjuvant and further pharmaceutically and/or veterinary acceptable carriers. Furthermore, a therapeutic or prophylactic composition according to the present disclosure can comprise more than one active ingredient in the form of an antigen.

The term “chromatography” shall be understood to refer to any kind of technique (preferably a preparative technique) which separates a virus particle of interest from other molecules present in a mixture by differential partitioning between a mobile phase and a stationary phase. The stationary phase is thereby preferably held in place, while the mobile phase moves in a definite direction. Partitioning is to be understood to occur repeatedly as the sample flows with the mobile phase along or through a dimension, e.g. length, of the stationary phase. As such, the term does not encompass a method, wherein a sample is and a stationary phase material are combined, mixed and incubated together and the supernatant is then recovered after the components (sample and stationary phase) could interact with each other (“batch adsorption”). In the context of the present invention column chromatography is preferred. “Column chromatography” as used herein is a technique in which a stationary phase material is contained in a container such as a tube or column having an inlet on one side of the container and an outlet on another side, preferably the opposite side. The stationary phase material may for example be comprised of particles of a solid stationary phase material or a support coated with a liquid stationary phase. It may fill the whole inside volume of the container (packed column) or be concentrated on or along the inside tube wall leaving an open, unrestricted path for the mobile phase in the middle part of the tube (open tubular column). In the context of the present invention, a packed column chromatography is most preferred.

It is understood that wherever aspects are described herein with the language “comprising”, “having”, “containing”, “involving” and similar expressions otherwise analogous aspects described in terms of “consisting of” and/or “consisting essentially of” are also provided.

The terms “derived”, “derived from”, “derivative” or “descendant” or “progenitor” as used herein in the context of either a host cell, a cell population or a virus particle according to the present invention relates to the descendants of such a host cell or virus particle which results from natural reproductive propagation including sexual and asexual propagation and, in the context of virus nucleic acids, the propagation of the virus genetic material by the host cell machinery. Propagation can lead to the introduction of mutations into the genome of an organism resulting from natural phenomena which results in a descendant or progeny, which is genomically different to the parental host cell, however, still belongs to the same genus/species and possesses the same characteristics as the parental host cell. A virus, derivative or descendant or progenitor can thus naturally possess one or more mutations. Such derivatives or descendants resulting from natural phenomena during reproduction or propagation are thus comprised by the term host cell or cell population according to the present disclosure and the skilled person can easily define, by means of molecular biology, microscopy or the like, that the derivative or descendant is indeed derived from a parental host cell of the same genus. These terms, therefore, do not refer to any arbitrary derivative, descendant or progenitor, but rather to a derivative, or descendant or progenitor phylogenetically associated with, i.e. based on, a parent cell or virus or a molecule thereof.

As used herein, the term “diafiltration” or “DF” is used to mean a specialized class of filtration in which the retentate is diluted with a (fresh) solution or buffer and re-filtered, to reduce the concentration of soluble permeate components. Diafiltration may or may not lead to an increase in the concentration of retained components, including, virus particles. For example, in continuous diafiltration, a solvent is continuously added to the retentate at the same rate as the filtrate is generated. In this case, the retentate volume and the concentration of retained components does not change during the process. On the other hand, in discontinuous or sequential dilution diafiltration, an ultrafiltration step is followed by the addition of solvent to the retentate side; if the volume of solvent added to the retentate side is not equal or greater to the volume of filtrate generated, then the retained components will have a high concentration. Diafiltration may be used to alter the pH, ionic strength, salt composition, buffer composition, or other properties of a solution or suspension of macromolecules.

The term “direct fluid communication” as referred to herein, implies that the fluid transported, e.g., between two unit operations, does not pass a section, e.g., another unit operation, where it is treated in such a way that its composition is substantially changed. In some embodiments of the present invention, a direct fluid communication is achieved through one or more flow lines, e.g., ducts arranged between two unit operations, which may optionally comprise bends, reservoirs, valves, and the like. Similarly, the term “direct” referred to in the context of subjecting a sample, liquid, mixture or the like to a unit operation, implies that the sample, liquid, mixture or the like does not pass a section, where it is treated in such a way that its composition is substantially changed.

An “excipient” is a substance included in a drug composition, including an immunological or a vaccine composition, which is added for the purpose of long-term stabilization, bulking up solid formulations that contain active ingredients, including, for example, infectious virus particles, or to confer a therapeutic enhancement on the active ingredient in the final dosage form, e.g. for improving the absorption, modifying the viscosity or for enhancing solubility.

The terms “flow-through mode” and “flow-through chromatography”, as used interchangeably herein, refer to a separation technique in which at least one product of interest (herein a virus particle) contained in a sample is intended to pass a chromatography medium, while the contaminants (here the one or more impurities) bind to the chromatography medium. The product of interest does not or only to a little extent interact with the chromatography medium and can thus be recovered in the flow-through.

As used herein, a “flow-through chromatography unit” refers to an apparatus comprising a stationary phase material and optionally a control unit, wherein the stationary phase material under appropriate conditions is capable of binding one or more impurities but not MV particles and wherein the control unit, if present, is preferably programmed to operate the chromatographic purification in flow-through mode. The control unit may in particular include commands to recover the MV particles containing flow-through and/or isolate it from the one or more impurities bound to the stationary phase material.

The terms “genetically modified”, “recombinant”, “genetically engineered” or similar expression as used herein refer to a nucleic acid molecule or an amino acid molecule or a host cell comprising a targeted and purposive manipulation and/or modification achieved by means of molecular biology or protein engineering, e.g. by introducing a heterologous sequence into another host cell, by modifying a naturally occurring nucleic acid sequences and the like. Further modifications include, but are not limited to, one or more point mutation(s), one or more point mutation(s), e.g. for targeted protein engineering or for codon optimization, deletion(s), and one or more insertion(s) of at least one nucleic acid or amino acid molecule, modification of an amino acid sequence, or a combination thereof. The terms can also imply a sequence, which per se occurs in nature, but has been purposively treated by means of molecular biology.

Percentages of homology or identity of nucleic acid or amino acid sequences shall be understood to correspond to values determined by using the EMBOSS Water Pairwise Sequence Alignments (nucleotide) program (http://www.ebi.ac.uk/Tools/psa/emboss_water/nucleotide.html) for nucleic acids or the EMBOSS Water Pairwise Sequence Alignments (protein) program (http://www.ebi.ac.uk/Tools/psa/emboss_water/) for amino acid sequences. Those tools provided by the European Molecular Biology Laboratory (EMBL) European Bioinformatics Institute (EBI) for local sequence alignments use a modified Smith-Waterman algorithm (see http://www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

As used herein, “impurities” and “contaminants” may interchangeably refer to undesired components in the viral preparation, which components may be present at any step during the purification process and particularly in the beginning of the downstream process. In some embodiments, impurities or contaminants may be host cells or fragments thereof, including host cell DNA and/or host cell proteins; viral fragments or viral nucleic acid; enzymes, such as BENZONASE® Nuclease, salts; and components of the cell culture medium.

An “immunogenic composition” as used herein refers to a composition which is able to induce an immune response in a subject. An immunogenic composition according to the present disclosure comprises at least one vaccine composition based on MV. A vaccine per se also is an immunological composition. However, it is well known to the skilled person that for being suitable to administration to an animal, an immunogenic composition can additionally comprise suitable pharmaceutically and/or veterinary acceptable carriers.

As used herein, the term “infectious” according to the present disclosure characterizes one or more virus particles that are able to (re-)infect a cell population, host cell or subject of interest, i.e. to enter a host and replicate therein and potentially spread to further cells or tissues.

“Load” as used herein denotes the part of a sample which is directly subjected to a chromatography step, i.e. loaded onto a chromatography unit.

The term “measles virus particles” (MV particles), as used herein, is to be understood as referring to any particle comprising or derived from MV including native (wild-type) or recombinant MV, either in live, attenuated, inactivated or killed form. Also covered by said term is a virion and a virus-like particle derived from, and thus comprising structural elements of, MV.

The term “MV titer” herein denotes the number of MV particles per volume. The various assays, which can be used for virus titer quantification include, but are not limited to, plaque assays, endpoint dilution assays, protein assays and transmission electron microscopy. Plaque-based assays are the standard method used to determine virus concentration in terms of infectious dose. Viral plaque assays determine the number of plaque forming units (pfu) in a virus sample, which is one measure of virus quantity. Endpoint dilution assays report 50% Tissue culture Infective Dose (TCID50) as measure of infectious virus titer. The endpoint dilution assay quantifies the amount of virus required to kill 50% of infected hosts or to produce a cytopathic effect in 50% of inoculated tissue culture cells. Protein-based virus quantification assays quantify either the amount of all protein or the amount of a specific virus protein in the sample rather than the number of infected cells or virus particles. Quantification most commonly relies on fluorescence detection. Exemplary protein-based virus quantification methods include hemagglutination assays, bicinchoninic acid assays and single radial immunodiffusion assay. TEM is a specialized type of microscopy that utilizes a beam of electrons focused with a magnetic field to image a sample. TEM images can show individual virus particles and quantitative image analysis can be used to determine virus concentrations. These high resolution images also provide particle morphology information that most other methods cannot. Quantitative TEM results will often be greater than results from other assays as all particles, regardless of infectivity, are quantified in the reported virus-like particles per mL (vlp/mL) result. An alternative method for determining the TCID is quantitative PCR (qPCR). The TCID₅₀ as used herein refers to median tissue culture infective dose as defined above. When reference is made herein to specific values of viral titers these are preferably determined by endpoint limit dilution assay, e.g. on Vero cells, and TCID₅₀ is be calculated by using the Kärber method.

As used herein, the term “prophylactic treatment” as referred to herein in the context of vaccine compositions means a treatment which mediates a protective immune response in a subject vaccinated so that there are no or less severe symptoms, when the subject after having been vaccinated and after having developed an immune response to the vaccine composition will encounter an infection with the non-attenuated wild-type strain corresponding to the virus antigens present in the vaccine composition. Similarly, a “prophylactic composition” as referred to herein denotes a composition which, when administered, mediates a corresponding protective immune response.

The terms “protection”, “protective immunity” and “protective immune response” refer herein to the ability of serum antibodies and cellular response induced during immunization to protect (partially or totally) against a virus of interest. Thus, an animal immunized by the compositions or vaccines of the invention will afterwards experience limited growth and spread if infected with the respective naturally occurring virus.

The term “purifying” as used herein refers to increasing the degree of purity of a product of interest (herein MV particles) from a mixture, composition or sample comprising the product and one or more impurities or contaminants. In some embodiments, the degree of purity of the product of interest is increased by removing (completely or partially) one or more impurities from the mixture, composition or sample.

The term “regulatory sequence” as used herein refers to a nucleic acid sequence which can direct and/or influence the transcription and/or translation of a target nucleic acid sequence of interest. The term thus refers to promoter and terminator sequences or to polyadenylation signals and the like.

The term “sample” herein implies a complex mixture or complex composition originating from a cell-based method, which mixture or composition comprises a product of interest (herein MV particles) and contaminants (herein one or more impurities). The cell-based method preferably includes at least one step of cultivating cells and a step of infecting the cells with MV. In some embodiments of the present invention, the sample is a cell culture supernatant or cell culture lysate directly obtained from a mixture comprising said supernatant and a residue containing cells and/or cell debris. In some embodiments, a sample results from clarification and/or a treatment with an agent having nucleic acid digesting activity of a cell culture supernatant or cell culture lysate directly obtained from a mixture comprising said supernatant and a residue containing cells and/or cell debris.

“Scaffold”, “virus scaffold” and similar expressions made in the context of MV shall be understood to refer to a framework or backbone structure based on a MV sequence, in particular a recombinant, infective and/or replicative MV particle, optionally expressing one or more foreign antigens, i.e. antigens that are not naturally occurring in wildtype MV. Therefore, the basic sequence of a MV scaffold will originate of a MV, whereas the sequence encoding the MV or the MV scaffold can comprise further elements.

The terms “sequence(s)” and “molecule(s)” are used interchangeably herein when referring to nucleic acid or amino acid sequences/molecules.

The term “sequentially” herein denotes events, which occur one after another. That is, in a series of sequentially events, only one event takes place at the same time. For example, event B starts with the ending of event A. After end of event B, the series may be complete, or another event (c) or again event A may take place.

As used herein, “simultaneously” encompasses precisely simultaneous as well as nearly simultaneous events. For example, simultaneous events may begin at about the same time, end at about the same time, and/or take place over at least partially overlapping time periods.

The term “stationary phase material” is interchangeably used with the terms “chromatography material” and “chromatography medium” and refers to any kind of a substance fixed in place for chromatography procedure. It is capable of separating a virus particle of interest from other molecules, in the context of the present invention in particular the one or more impurities, present in a mixture. The stationary phase material can be classified according to its (primary) mode of interaction. Examples, without limitation, of such material comprise: size exclusion media, ion exchange media; anion exchange media; cation exchange media; hydroxyapatite media; hydrophobic interaction chromatography media; and mixed-mode media. Suitable stationary phase materials include, without limitation, a non-aqueous matrix comprising agarose, sepharose, glass, silica, polystyrene, collodion charcoal, sand, polymethacrylate, cross-linked poly(styrene-divinylbenzene), agarose with dextran surface extender or any other suitable material, which can optionally be functionalized.

As used herein, the term “ultrafiltration” or “UF” refers to any technique in which a liquid (e.g., a solution or suspension) is subjected to a semi-permeable membrane that retains macromolecules (herein MV particles) while allowing solvent and small solute molecules to pass through. Ultrafiltration may be used to increase the concentration of macromolecules in a liquid and/or to decrease the concentration of impurities, i.e. improve purity. It may employ membranes with molecular weight cut-off (MWCO) ranging from 0.5 to 1,000 kDa. The MWCO is defined as the minimum molecular weight at which 90% of the solute is retained by the membrane. The rejection profiles may be generally determined by polymer dextrans in the range of 1-2000 kDa. Ultrafiltration membranes may be composed by two main layers, e.g., a thick macroporous support that provides mechanical strength, and a thin skin layer that is responsible for membrane selectivity and permeability. UF membranes can be manufactured using different polymers, such as regenerated cellulose (RC), polysulfone (PS), modified polyethersulfone (mPES), and polyvinylidene fluoride (PVDF).

As used herein, the term “ultrafiltration/diafiltration” or “UF/DF” refer to a process, technique or combination of techniques that accomplishes ultrafiltration and diafiltration, either sequentially or simultaneously. In the context of the present invention, UF/DF preferably occurs in the same UF/DF unit.

The term “vector” or “plasmid vector” as used herein defines a system comprising at least one vector suitable for transformation, transfection or transduction of a host cell. A vector per se thus denotes a cargo for the delivery of a biomolecule into a host cell of interest, wherein the biomolecule includes a nucleic acid molecule, including DNA, RNA and cDNA, or, in the case of a transfection system as vector, an amino acid molecule, or a combination thereof. A preferred vector according to the present invention is a plasmid or expression vector. An expression vector can comprise one vector encoding at least one target molecule, preferably a nucleic acid molecule, to be introduced into a host cell. A vector of the vector system can also comprise more than one target molecules to be introduced. Alternatively, the vector system can be built from several individual vectors carrying at least one target molecule to be introduced. An expression vector additionally comprises all elements necessary for driving transcription and/or translation of a sequence of interest in a host cell, the expression vector is designed for. These elements comprise, inter alia, regulatory elements, which are involved in the regulation of transcription, including promoters and the like functional in the host cell of interest. Furthermore, an expression vector comprises an origin of replication and optionally depending on the type of vector and the intended use a selectable marker gene, a multiple cloning site, a tag to be attached to a sequence of interest, a chromosomal integration cassette and the like. The choice and possible modification of a suitable expression vector for use with a respective host cell and sequence of interest to be inserted into the expression vector is well within the capabilities of the person skilled in the art.

A “viral particle” or “virus particle” as used herein refers to a single particle derived from a viral nucleic acid, which is located outside a cell. As the viral particle may contain genetic information, it may be able to replicate, and/or propagate in a susceptible host cell. An “infectious virus particle” represent the mature and infectious form of a virus.

The term “viral vaccine” or “vaccine composition” as used herein refers to a virus particle, which is able to induce a protective immune response in a subject.

A “virion” as used herein refers to a single particle derived from a viral nucleic acid, which is located outside a cell containing nucleic acids and thus being able to replicate or to be transcribed in a suitable host cell.

A “virus-like particle” or “VLP” as used herein refers to at least one virus particle, which does not contain nucleic acid. VLPs can thus be used for vaccination or inducing an immunogenic reaction in a subject. Due to the absence of nucleic acids they will, however, not be able to replicate in a host cell and are thus non-replicative.

A “virus sample”, “virus material” or the like refers to a material comprising at least one of a (recombinant infectious) virus particle and/or a virion and/or a VLP.

The term virus stock refers to a seed stock comprising at least one recombinant infectious virus particle derived from a MV scaffold suitable to infect a host cell of interest. Given the fact that a virus should be provided in a certain amount to efficiently infect a host cell of interest, the term virus stock usually implies a stock comprising more than one infectious virus particle, as depending on the host cell of interest, the infectious virus particle of interest and the intended multiplicity of infection (MOI) used for infection. The MOI usually depends on the Tissue culture infective dose (TCID). An appropriate MOUTCID₅₀ can be determined following common tests, e.g. the Kärber method or the Reed Muench method. A virus stock as used herein preferably refers to a recombinant virus stock.

“Viscosity” is a measure of a fluid's resistance to flow and can be measured at 25° C. by DV-II+Pro viscometer (Brookfield Engineering Laboratories, Middleboro, Mass., USA). Shear stress and corresponding shear rate data can be used for evaluation of viscosity by power-law according to Ostwald-de Waele. Viscosities measured at a shear rate of 525 1/sec can be selected for comparison. Whenever the present disclosure refers to viscosity values these shall be understood to be determined in accordance with the aforementioned test unless otherwise stated.

By “recombinant virus (particle)” is meant a virus that has been genetically altered, e.g., by the addition or insertion of a heterologous nucleic acid construct into the particle. A recombinant virus does preferably not include a virus as they exist in nature. Still, obtaining a cDNA from a naturally occurring virus and making this sequence recombinantly available also shall imply a recombinant virus (particle) according to the present disclosure.

“Recovery” is interchangeably used herein with the term “yield” and refers to the percentage recovered from a unit operation, relative to amount subjected to said unit operation. For example, a recovery of 100% TCID50 obtained for flow-through chromatography indicates that the flow-through has the same infectious dose as the load.

As used herein, all numerical values or numerical ranges include integers within such ranges and fractions of the values or the integers within ranges unless the context clearly indicates otherwise. Thus, to illustrate, reference to at least 1-10% identity, includes 1%, 2%, 3%,4%, 5%, 6%, 7%, 8%, 9%, 10%, as well as 1.1%, 1.2%, 1.3% 1.4%, 1.5%, etc., 2.1%, 2.2%, 2.3%, 2.4%, 2.5%, etc., and so forth.

Reference to a number with more (greater) or less than includes any number greater or less than the reference number, respectively. Thus, for example, a reference to less than 40,000, includes 39,999, 39,998, 39,997, etc. all the way down to the number zero; and less than 100, includes 99, 98, 97, etc. all the way down to the number zero.

As used herein, all numerical values or ranges include fractions of the values and integers within such ranges and fractions of the integers within such ranges unless the context clearly indicates otherwise. Thus, to illustrate, reference to a numerical range, such as 2,000-40,000 includes 2,000; 3,000; 4, 000; 5,000, 6,000, etc. as well as 2,100; 3,100; 4,100; 5,100; 6,100; etc., and so forth. Reference to a range of 20-100 therefore includes 21, 22, 23, 24, 25, 26, 27, 28, 29, 30, 31, 32, 33, 34, 35, 36, 37, 38, 39, 40, etc., up to and including 100, as well as 21.1, 21.2, 21.3, 21.4, 21.5, etc., 22.1, 22.2, 22.3, 22.4, 22.5, etc., and so forth.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Unless defined otherwise or dictated otherwise by context, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

### DETAILED DESCRIPTION

It is a common hurdle in the field of virology and vaccination that viruses possess highly different and divergent biological and biochemical properties and therefore purification schemes must be established specifically for each virus particle. Virus genomes generally show a high degree of variability. In general, they can be composed of DNA, single or double stranded, or RNA, as plus or minus strand or ambisense, the genome can be linear, circular or segmented and there can be an envelope (composed of lipids and proteins) or not. Besides that, the genome size can vary a lot from about 1.7 kilobase (kb) (e.g. Circoviridae or Hepatitis delta virus) to about 2.5 megabase (Mb) (e.g. Pandoravirus salinus). With respect to purification, especially the size, diameter and chemical reactivity of the exposed surface of a virus particle, especially of the envelope, if present, are factors to be taken into consideration. The challenges in producing a virus particles-containing composition for pharmaceutical use not only imply the provision of a suitable vaccine vector and propagation strategies as detailed further below, but further demand the provision of improved methods for down-stream processing of the particular virus material of interest to achieve a significant increase in purity of the resulting material without a loss in functional, i.e. immunogenic, virus particles.

Clinical batches of MV may be produced in Vero cells or other suitable cell lines known to the skilled person adapted to serum-free growth in cell factory multilayer vessels, resulting in 50% of the virus in the supernatant and 50% staying associated with the cells. The supernatant is clarified by filtration and treated with Benzonase to digest contaminating nucleic acids. MV particles are then concentrated and purified using ultrafiltration and diafiltration, followed by a final passing through a clarifying filter. Especially regarding purity considerations, vaccine candidates based on MV particles suffer from the drawback that they still contain a significant amount of impurities. Though the amounts might be acceptable for vaccines, OV therapy due to a high dose and the route of administration demand much higher purities and yet high yields.

To this end, the present invention provides for a method suitable for purifying MV particles, a system for carrying out said method as well as compositions that are suitable for being used in therapy and prophylaxis obtained thereby. Moreover, various uses of the compositions in therapy are disclosed.

Accordingly, in a first aspect of the present invention, there is provided a method for purifying MV particles from a sample, the method comprising in sequential order the following steps: (i) loading a sample containing MV particles and one or more impurities onto a stationary phase material and carrying out flow-through chromatography to bind at least a fraction of the one or more impurities contained in the sample and to produce a flow-through comprising at least a fraction of the MV particles contained in the sample; and (ii) carrying out filtration, preferably ultrafiltration, and obtaining a retentate having an increased MV titer relative to the MV titer comprised in the flow-through.

According to the inventive concept disclosed herein, a concentration step is carried out after a chromatography step, wherein the chromatography step is operated specifically in flow-through mode. Since samples such as cell culture supernatants originating from virus infected host cell usually contain virus particles in large dilution—which is all the more true when producing recombinant virus particles—it has been generally thought that for an economic process one of the very first steps of downstream processing has to be a volume reduction step (Nestola et al., supra). This sequence of steps combined with chromatography to be carried out specifically in flow-through mode as defined in the first aspect of the invention thus represents a complete departure from previous practice for purifying MV particles.

A big advantage of operating the chromatography in flow-through mode is that it avoids the drawback that large viruses such as MV can often not diffuse into the pores of commercially stationary phase materials formed into a packed bed or as monoliths (e.g., for a bind-elute modus of operation). Purification therefore will lead to low recoveries, when the chromatography step is carried out in conventional bind-elute mode. Further elucidation of this observation lead to the hypothesis that binding of MV particles to the stationary phase material increases back pressure to an unacceptable extent. MV must be treated as >1 μm particles that are extremely shear sensitive, in order to maximize recoveries and retain infectivity. Without being bound by theory, it is thought that the large size of MV particles, which, when bound to a stationary phase material contained in a column, strongly increase the backpressure, even when the sample has not been concentrated before.

Due to viscosity increase, this situation would be further complicated, when the sample was concentrated as a very first step of downstream processing, i.e. before chromatography. In fact, known purification protocols of MV particles, to the best of the inventor's knowledge, do not comprise a packed bed chromatography step at all. This is a severe drawback, in particular in the context of providing highly pure and concentrated compositions for OV therapy, since chromatography is a potent purification technique, generally allowing high recoveries and high purity.

These facts and advantages of the strategy of the present invention may be further explained by the following general formula illustrating the importance of viscosity consideration in fluid mechanics and its influence on the shear stress:

τ=η×\dot{γ}

wherein τ is the shear stress, \dot{γ} is the shear rate, and η is the apparent viscosity (constant only for Newtonian fluids). In fluid mechanics, for a Newtonian fluid, the shear stress, τ, on a unit area moving parallel to itself, is generally found to be proportional to the rate of change of velocity with distance perpendicular to the unit area (see Bird et al., Transport Phenomena, Wiley, 1960).

In a column, the back pressure of the column and the shear stress are thus only correlated by the viscosity of the fluid. For example, during elution in a bind-elute mode, viscosity (and in turn the shear stress) will thus increase, as concentration increases (particularly when using monolithic columns, as packed bed settings will likely not increase that high concentrations).

In comparison to the method disclosed in WO 2017/109211, higher capacities are achieved by the flow-through chromatography step comprised in the method of the invention than in a monolithic column configuration operated in bind-elute mode. It was observed that the more virus was bound to the monolith column, the stronger the pressure increased. In some cases, the pressure increased to such extent that a “bypass” was formed in the column leading to (1) breakthrough of viruses (the theoretical capacity of the column cannot be used) and (2) widening of elution peaks, i.e. dilution of the product. In contrast, when the chromatography step is operated in flow-through mode, impurities selectively bind to the stationary phase material, while the MV particles run there through. This mode thus allows influencing viscosity and thus shear stress in a highly favorable manner during the whole chromatography step. These conditions in turn are favorable for both the purity and the infectivity of the MV particles.

Another advantage is that the various steps comprised by the method of the present invention can be carried out aseptically under GMP conditions throughout the process resulting in a purified and concentrated virus preparation that does not require sterile filtration. In the production of pharmaceutical compositions, such as vaccine compositions, for the administration to subjects, sterility and safety of the composition must be ensured. Sterility of such compositions is typically achieved by means of sterile filtration, i.e., though a 0.2 μm filter membrane, prior to administration. For compositions comprising relatively large viruses (i.e., approximately 100 nm or larger), sterile filtration may result in a substantial loss of virus due to it being retained on the membrane, reducing the viral yield from the purification process. Additionally, some viruses, such as MV are sensitive to shear stress, which pose high requirements to a purification process. Since each step of the purification process must in this case be performed under gentle conditions, alternative sterilization methods are rendered useless. Hence, it is preferred that the unit operations of the method and the various embodiments of the invention are carried out aseptically.

As a further advantage, the various steps comprised by the method of the present invention of the invention allow to be integrated and carried out continuously. In a real continuous process, a product is manufactured un-interrupted in time. In theory the mass flow is constant over time. Process disturbance change the mass flow over time, but to such an extent the process is still considered fully continuous. In a quasi or pseudo continuous process, unit operations are operated in a cyclic manner. The mass flow changes/fluctuates over time. In an integrated process the outflow of one unit operation is directly fed in the next one without any hold step in between. Sometime surge tanks are put between unit operations to compensate for different flow rates. Further advantages include relatively high flow rates and thus rapid processing via flow-through chromatography.

Suitable buffers for use in the flow-through chromatography step include HEPES, Sodium phosphate, Tris, Triethanolamine, BICINE, bis-Tris, or diethanolamine, preferably HEPES, or other comparable buffer systems known to the skilled person. Depending on the functionality of a stationary phase material used in the flow-through chromatography unit, the pH value and conductivity is set so that the at least one impurity bind to the stationary phase material, whereas the MV particles do not in order to allow separation. If the stationary phase material has/have size-exclusion functionality and/or hydrophobic interaction functionality, the pH may range between pH 6.0 and 9.0, preferably between pH 7.0 and 8.0. In the context of using Capto Core 700 stationary phase material, the load, i.e. the MV particles-containing sample subjected to flow-through chromatography, comprises HEPES (or another of the above cited buffers or buffer systems based thereon), preferably in the range of 10 to 200 mM, more preferably in the range of 10 to 150 mM and most preferably in the range of 20 to 100 mM, and optionally NaCl, which is preferably present in an amount of 20 to 300 mM, more preferably in an amount of 50 to 250 mM and most preferably in an amount of 100 to 200 mM. The pH value in this case is preferably between pH 6.0 and 9.0, more preferably between pH 6.5 and 8.5 and most preferably between pH 7.0 and 8.0.

Impurities arising from virus particle manufacturing processes include process related impurities and product related impurities, in particular including, but not restricted to, host cells, cell debris, host cell proteins, or lipids, polysaccharides, and nucleic acids stemming from the host or virus particles and virus proteins, protein contaminants, either resulting from cell culture additives or from enzymes added during cultivation and processing, e.g. bovine serum albumin, if no serum-free approach is chosen, or endonucleases added for nucleic acid digestion, microcarriers used for host cell cultivation, or foreign nucleic acids neither belonging to the host cell nor the MV particles of interest. Preferably the at least one impurity is selected from the group consisting of host cell proteins, nucleic acids and in particular both host cell proteins and nucleic acids.

To obtain a retentate having an increased MV titer relative to the MV titer comprised in the flow-through, the MV particles are retained by the filter, while the liquid, preferably along with further impurities, is allowed to pass through the filter. Therefore, in the context of the present invention, step (b) defines a filtration method, wherein the product of interest is retained in the retentate while liquid, and preferably impurities, are allowed to cross to the permeate side.

An advantage of the present inventive concept is that method according to the present invention is suited for integrated manufacturing and/or quasi, semi or fully continuous processing. This is of great importance for large virus particles such as MV because the product cannot be subjected to a sterile filtration before filling. A fully continuous process manufactures the product un-interrupted in time. In theory the mass flow is constant over time. Process disturbance change the mass flow over time, but in such the process is still considered fully continuous. In a quasi or pseudo continuous process, unit operations are operated in a cyclic manner. The mass flow changes/fluctuates over time. In an integrated process the outflow of one unit operation is directly fed in the next one without any hold steps. Sometime surge tanks are put between unit operations, e.g. to compensate for differing flow rates occurring in two unit operations being in direct fluid communication.

Further advantages of carrying out filtration step (ii) after flow-through chromatography step (i) include the possibility to increase purity to a higher degree; and decrease filter area, as compared to a corresponding method, wherein filtration is carried out first. More specifically, it was observed that tangential flow filtration (TFF) carried out with clarified and endonuclease treated cell culture supernatant was strongly affected by rapid fouling of the membrane. This results in a lower capacity, a longer duration of the process and, hence, an inefficient process. These limitations could be completely avoided by carrying out filtration after flow-through chromatography step (i). It was further found that, in stark contrast to the method according to the first aspect of the present invention, purity could not be increased by TFF, if it was carried out with clarified and endonuclease treated cell culture supernatant. Moreover, the sequence of steps enables the concentration of MV particles in the retentate, whereas this is not achieved when carrying out TFF at the beginning of the downstream process. Last but not least, filtration step (ii) can be easily implemented with a buffer exchange step. Thereby, filtration step (ii) enables to provide MV particles in a final formulation and shorten the number of process steps.

A successful MV purification platform should further contain an upstream processing which is based on defined culture media with a virus titer and a downstream processing which is suited for integrated manufacturing, because the product cannot be subjected to a sterile filtration step.

In preferred embodiments of the present invention, there is thus provided a method according to the first aspect of the invention and the various embodiments thereof, wherein the sample is obtained by a method comprising one or more of the following steps: (a) infecting at least one host cell with a virus stock comprising at least one MV particle; (b) incubating the at least one host cell infected with the virus stock to allow virus production; (c) obtaining a cell culture supernatant containing MV particles and one or more impurities; (d) clarifying the cell culture supernatant to obtain a clarified cell culture supernatant. More specifically, the sample is preferably obtained by a method comprising at least step (c) obtaining a cell culture supernatant containing MV particles and one or more impurities. More preferably, the sample is obtained by a method comprising at least steps (c) obtaining a cell culture supernatant containing MV particles and one or more impurities and (d) clarifying the cell culture supernatant to obtain a clarified cell culture supernatant. Most preferably, the method by which the sample is obtained comprises all the steps (a) to (d) as defined above.

The at least one host cell shall be understood to refer to one host cells, preferably more than one host cells and most preferably a plurality of host cells. The at least one host cell, and preferably plurality of host cells, can be obtained by cultivating one or a population of host cells, for example originating from a cell bank, to a larger population of host cells. In preferred embodiments, the at least one host cell comprises or consists of cells selected from the group consisting of a variety of established vertebrate, preferably mammalian, or invertebrate cells lines, including Vero cells (African green monkey kidney cells), chicken embryo fibroblast cells, HEK293 cells, WI-38 cells, Madin Darby canine kidney (MDCK) cells, HeLa cells, BJAB (Human Burkitt's Lymphoma), Caco-2, CAL-148, hMSC-TERT (human bone marrow derived mesenchymal stem cell), MIA-PaCa-2, SCLC-21H, RMPI-8226, and MRC5 cells, preferably Vero or MCR-5 cells, but also designer cells for viral vaccine production, including, for example, PER.C6, CAP, AGE1.CR, AGE1.CR.pIX, EB66 cells, PBS-1, QOR/2E11, SogE, MFF-8C1 cells and the like, which are publicly available.

One of the key parameters in process design for anchorage dependent cells is the availability of a robust and scalable seed train. Using classical T-flasks puts a high work load to the operators of such a process and the usage of stirred formats such as spinner flasks and seed bioreactors in combination with microcarrier bear severe problems caused by bead-to-bead transfers. To overcome these problems, in a preferred embodiment of the present invention, a seed train that is fully based on static cultures using multi-layer flasks, preferably in different formats, is used to produce a plurality of host cells, which is then infected as defined above in step (a). In this context, the expression “fully based on static cultures” is to be understood as a culture, wherein the cells for inoculation are cells cultivated, e.g., in flasks, in a static culture in contrast to cells cultivated in a stirred system.

Due to the adherent nature of Vero cells a bioreactor usually needs to provide specific surfaces for the cells to grow on. Such surfaces can be provided by way of fiber-cell matrices or different types of microcarrier. Microcarrier have in the past been used for cell culture processes and they have been shown to be suitable for the cultivation of Vero cells even at large scales. In the context of the present invention, it is preferred that a microcarrier is employed in the production, and preferably also in the infection step (a) and/or incubation step (b), of the at least one host cell and preferably a plurality of host cells. By using a microcarrier cells readily attach thereto within 1 hour after inoculation and continue growing upon confluence. A specifically preferred microcarrier is comprises or consists of dextran beads, preferably having a particle size of 10 μm to 500 μm, preferably 20 μm to 300 μm, more preferably 30 μm to 200 μm and most preferably 50 μm to 100 μm. Such microcarriers are available under the trade name Cytodex® 1. The microcarriers are preferably contained in the bioreactor in an amount of 1 to 20 g/L, more preferably in an amount of 1.5 to 6 g/L and most preferably in an amount of 2 to 4 g/L. In a preferred embodiment, host cells are inoculated in a density of 1×10³ to 1×10⁵ cells/cm² microcarrier surface, preferably 2×10³ to 5×10⁴ cells/cm² microcarrier surface, more preferably 5×10³ to 2×10⁴ cells/cm² microcarrier surface and most preferably about 1×10⁴ cells/cm² microcarrier surface. Using lower numbers would be beneficial regarding seed-train steps but the number of microcarrier not covered with cells increases to a level that reduces the overall productivity of the process.

Step (a) involves infecting at least one host cell with a virus stock comprising at least one MV particle. In a preferred embodiment, the at least one host cell is infected with a Multiplicity Of Infection (MOI) of 0.0001 to 0.1, preferably 0.0002 to 0.005 and more preferably 0.0005 to 0.002 and most preferably 0.001±50%. As it is known to the skilled person, the MOI will have to be and can easily be determined for a host cell as well as a virus stock of interest. In order to preserve infectivity of the virus particles, temperature is preferably maintained at 32.5±3° C., more preferably 32.5±2° C. and most preferably 32.5 ±1° C.

Step (b), incubating the at least one host cell infected with the virus stock to allow virus production, typically involves cultivation of the at least one host cell under suitable conditions. For example, it/they can be grown under the same conditions, which were used for infection. In some cases, the at least one host cell undergoes autolysis. In this case, MV particles containing cell culture supernatant is directly obtained. In other cases, step (b) may comprise cell lysis, i.e. an active step, wherein the at least one host cell is lysed. This can be achieved by adding a chemical agent having cell lysis activity and/or exercising mechanical force such as high pressure on the at least one host cell.

The method according to the various embodiments of the invention, may further comprise: determining viable cell density. Monitoring total cell density is generally a reliable method for measuring cell growth. The most relevant information is obtained during the lag and growth phase before significant cell death occurs. When virus particles are produced using host cells attached to a microcarrier, it is however difficult to determine cell growth on-line. However, a lack of on-line data limits the possibility to understand and control the process in detail. It has now been surprisingly found that a probe can determine viable cell density based on permittivity measurements, even when the cells are attached to the microcarrier.

Hence, in a preferred embodiment, the production of the at least one host cell, preferably a plurality of host cells and/or step (a) and/or (b) comprises determining viable cell density and optionally total cell density. The viable and/or cell density determination is preferably based on (an) on-line technique(s). An exemplarily, and herein preferred, technique for on-line determination of viable cell density is based on capacitance. In an alternating electrical field, viable cells behave like small capacitors. The charge from these small capacitors is measured by a sensor and reported as permittivity (capacitance per area). A big advantage of such determination is that it allows on-line determination in real-time and gives a user a deep insight on cell culture or fermentation. Moreover, it is insensitive to microcarriers and cell debris. The total cell density determination may be based on on-line measurements including turbidity and optical density measurement at NIR (Near Infra-Red) wavelengths, or any other suitable measurement, whether allowing on-line or off-line determination of total cell density.

Using data provided by the viable cell probe, it is possible to precisely determine the ideal infection and harvest time points. A great advantage of such a principle is that only live cells are measured, so that the measurement is deteriorated neither by cellular debris nor by the microcarrier. A decrease of the signal thus correlates with a decrease in the total amount of viable cells in the system. Under the prerequisite that all process parameters are within defined specifications, the first decrease of permittivity signals in the course of infection can therefore be defined as ideal point of infection. Similar considerations can be made for harvesting the culture. If the signal falls below a certain threshold most of the cells are dead and the culture is ready for further processing. Predicting the ideal harvest time point is crucial for further downstream processing regarding highest possible yields and lowest possible contaminations due to lysed cells.

Step (c) obtaining a cell culture supernatant containing MV particles and one or more impurities may follow a procedure, wherein the cell culture supernatant is recovered. In such procedure, the microcarriers may be let to settle down, for example for at least 15 min, before withdrawing the cell culture supernatant. The cell culture supernatant may then be subjected to step (i) as defined above, or to one of the following steps (d) or (e). Optionally, the use of a harvestainer for separation of microcarrier beads may be employed. In certain embodiments, microcarrier free culture may be preferred depending on a host cell of interest.

For the purpose of the present invention, the bulk product consists of the virus particles produced in and released from a plurality of host cells (i.e. a cell population) or from a host cell. Clarification aims at removing cells and cell debris from the host cells infected with and producing a virus. Clarification usually does not include separation of impurities that have a size corresponding to the virus particles to be purified or smaller. Common methods for clarification are centrifugation and microfiltration, including tangential flow filtration (TFF), ultracentrifugation, dead end filtering and the like. In the context of clarifying a bulk product comprising MV particles centrifugation is less desired, as centrifugation processes are not easily scalable under GMP conditions.

Step (d), clarifying the cell culture supernatant to obtain a clarified cell culture supernatant, is therefore preferably accomplished by a method other than centrifugation, more preferably by means of dead end filtration, including inter alia depth filtration and/or membrane filtration, and most preferably by depth filtration. A single filter may be used, or a filter cascade comprising two or more filters connected in series may be employed for the purpose of the present invention. A preferred embodiment can rely on a single filter of 3 μm. In another embodiment in the context of the present invention, a filter cascade is used comprising or consisting of a first and a second filter, wherein the first filter has preferably a nominal pore size of 10 to 200 μm, more preferably 20 to 150 μm, even more preferably 30 to 120 μm and most preferably 40 to 100 μm. The second filter has preferably a nominal pore size of 0.5 to 20 μm, more preferably 0.8 to 10 μm and most preferably 1 to 5 μm. In a particular embodiment, the first filter has a nominal pore size of 40 to 100 μm and the second filter has a nominal pore size of 1 to 5 μm. In another particular embodiment, a filter cascade comprising or consisting of a first 50 μM filter and a second 3 μM filter is used. It is to be understood that the second filter comes after the first filter, i.e. is arranged downstream of the first filter, and preferably that the first and the second filter are in direct fluid communication with each other. Single filter embodiments may be preferred for practical reasons (economic concerns, yield etc.).

Any other method known to the skilled person and suitable to perform a crude separation of the host cellular material and the supernatant comprising the virus particles of interest are also comprised by the term “clarifying” or “clarification”. As discussed herein, TFF suffers from certain drawbacks when performed at an early stage of the process. Hence, in preferred embodiments, clarification does not comprise TFF and/or a volume reduction step associated with an increase of viscosity.

To reduce or eliminate contaminating nucleic acids, the cell culture supernatant obtained in step (c) as defined above, or the clarified cell culture supernatant obtained in step (d) as defined above, can be treated with an agent having nucleic acid digesting activity. Accordingly, in a further embodiment of the present invention, the method additionally includes step (e) treating the cell culture supernatant obtained in step (c) as defined above, or the clarified cell culture supernatant obtained in step (d) as defined above, with an agent having nucleic acid digesting activity. The agent is preferably an endonuclease such as Benzonase. Benzonase is a genetically engineered endonuclease from Serratia marcescens. The enzyme is a dimer of 30 kDa subunits with two essential disulfide bonds. This endonuclease attacks and degrades all forms of DNA and RNA (single stranded, double stranded, linear and circular) and is effective over a wide range of operating conditions. The optimum pH for enzyme activity is found to be 8.0-9.2. It completely digests nucleic acids to 5′-monophosphate terminated oligonucleotides 3 to 5 bases in length. This is ideal for removal of nucleic acids for applications where complete digestion of nucleic acids is desirable. It also reduces viscosity in protein extracts and prevents cell clumping. Treatment of the (clarified) cell culture supernatant is preferably carried out by Benzonase in the presence of a magnesium source such as magnesium chloride.

After addition of the agent having nucleic acid digesting activity to the cell culture supernatant obtained in step (c) as defined above, or the clarified cell culture supernatant obtained in step (d) as defined above, it can be incubated under conditions which allow for at least 50% removal of nucleic acids, preferably at least 60% removal of nucleic acids, more preferably at least 70% removal of nucleic acids, even more preferably at least 80% removal of nucleic acids and most preferably at least 90% removal of nucleic acids. The term “removal of nucleic acids” as used herein means that the nucleic acids are digested to 5′-monophosphate terminated oligonucleotides less than 5 bases in length. Typical conditions include incubation at 20° C. to 37° C. for up to 24 hours. The higher the temperature the shorter is usually the incubation time provided that the same amount of agent having nucleic acid digesting activity is used. For example, the sample can be incubated at 20 to 25° C. for about 12 to 24 hours, or at 30 to 37° C. for about 0.5 to 4 h. Afterwards, the treated cell culture supernatant can be further processed, e.g. by directly loading onto the stationary phase material and carrying out flow-through chromatography.

According to a specific embodiment, the cell culture supernatant obtained in step (c) as defined above, or the clarified cell culture supernatant obtained in step (d), is incubated for 1 h at 37.0±1.0° C. and preferably mixed. The required amount of Benzonase and magnesium chloride to obtain a final concentration of 50 U/mL Benzonase and 2 mM magnesium chloride (as a cofactor) are mixed together into a homogenous solution and then aseptically added to the (clarified) cell culture supernatant obtained in step (c) or (d), and the solution is preferably mixed. The (clarified) cell culture supernatant is then incubated at 37.0±1.0° C., 50 rpm for 1-2 hours. Afterwards, the (clarified) cell culture supernatant is removed from the incubator. To stop or at least slow down the endonuclease, preferably the Benzonase, EDTA may be added, preferably in an amount to obtain a final concentration of 5 mM EDTA.

If both steps (d) and (e) are encompassed by the method of the present invention, they can be operated either in consecutive order, (d)→(e), or vice versa, (e)→(d). After the sample has been obtained by a method that comprises at least step (c) as defined above, or at least steps (c) and (d) as defined above, or at least steps (c), (d) and (e) as defined above, the sample is, preferably directly, subjected to the flow-through chromatography step (i) as defined above.

The method according to the various embodiments of the invention, may further comprise: determining the concentration of contaminating nucleic acids and/or the amount of total protein and/or the amount of host cell proteins and/or virus titer, e.g. of the load and/or the flow-through and/or the retentate. In certain embodiments, the method may alternatively, or additionally, comprise determining viscosity, in particular of the load.

Methods to determine the concentration of contaminating nucleic acids such as host cell DNA are known to the person having skill in the art. A standard method for detecting small amounts of contaminating DNA in a sample is quantitative real-time PCR (qPCR or qRT PCR) (e.g. PicoGreen® assay (Life Technologies)). Another method for detecting contaminating DNA or proteins in a sample of interest are threshold DNA assays (e.g. Threshold® Immunoligand Assay (ILA) or Threshold® Total DNA Assay Molecular Devices). Said methods both show a high sensitivity and a good detection limit in the pictogram range and are readily available to the skilled person.

Likewise, methods for performing quantification of total protein, or of specific proteins contained as contaminants in a sample or a composition comprising the purified virus particles can be quantified by methods including a BCA (bicinchoninic acid) assay or a Vero cell host cell protein (HCP) ELISA assay (Cygnus Technologies, current detection limit as declared by the manufacturer: 700 pg/mL) or other enzyme and/or fluorescence based methods. Said methods are readily available to the skilled person. The various assays, which can be used for virus titer quantification include, but are not limited to, plaque assays, endpoint dilution assays, protein assays and transmission electron microscopy.

Plaque-based assays are the standard method used to determine virus concentration in terms of infectious dose. Viral plaque assays determine the number of plaque forming units (pfu) in a virus sample, which is one measure of virus quantity. Endpoint dilution assays report 50% Tissue culture Infective Dose (TCID50) as measure of infectious virus titer. The endpoint dilution assay quantifies the amount of virus required to kill 50% of infected hosts or to produce a cytopathic effect in 50% of inoculated tissue culture cells. An alternative method for determining the TCID is quantitative PCR (qPCR). The TCID₅₀ as used herein refers to median tissue culture infective dose as defined above. When reference is made herein to specific values of viral titers these are preferably determined by endpoint dilution assay, e.g. on Vero cells, and TCID₅₀ is be calculated by using the Kärber method.

In a further embodiment of the present invention, there is provided a method according to the first aspect of the invention and the various embodiments thereof, wherein the sample loaded onto the flow-through chromatography unit has a MV titer less than 5 times higher, preferably less than 4 times higher, more preferably less than 3 times higher, even more preferably less than 2 times higher and most preferably less than 1.5 times higher, than the MV titer in the cell culture supernatant obtained in step (c) as defined above or in the clarified cell culture supernatant obtained in step (d) as defined above. In some embodiments, this may mean that there occurs no concentration step between said step (c), or said step (d), and said step flow-through chromatography step (i) as defined further above, or at least no concentration step that increases MV titer 5-fold or more, preferably 4-fold or more, more preferably 3-fold or more, even more preferably 2-fold or more and most preferably 1.5-fold or more. In some embodiments, there occurs no volume reduction step between said step (c), or said step (d), and said step flow-through chromatography step (i) as defined further above, or at least no volume reduction step that decreases the volume by 5-fold or more, preferably 4-fold or more, more preferably 3-fold or more, even more preferably 2-fold or more and most preferably 1.5-fold or more. In addition to or alternatively to the preceding embodiment, the sample loaded onto the flow-through chromatography unit has a host cell protein content of at least 50%, preferably at least 60%, more preferably at least 70%, even more preferably at least 80% and most preferably at least 85% relative to the host cell protein content in the cell culture supernatant obtained in step (c) or in the clarified cell culture supernatant obtained in step (d). In some embodiments, this may mean that the cell culture supernatant obtained in step (c) or the clarified cell culture supernatant obtained in step (d) is directly loaded onto the flow-through chromatography unit. In some embodiments, there is no host cell removal step arranged between said step (c), or said step (d), and flow-through chromatography step (i).

Advantageously, the method according to the first aspect of the present invention is not based on a concentration step at an early step of downstream processing. In fact, the best results in terms of yield (recovery) and purity are achieved if no ultrafiltration step is carried out before the flow-through chromatography in order to keep viscosity low and thus avoid a potentially harmful pressure increase during chromatography.

In accordance with this observation, a further embodiment of the present invention provides a method according to the first aspect of the invention and the various embodiments thereof, wherein the sample containing MV particles as defined in the method of the first aspect of the present invention, or the cell culture supernatant obtained in step (c) as defined above, or the clarified cell culture supernatant obtained in step (d) as defined above, or the treated cell culture supernatant obtained in step (e) as defined above, is directly loaded onto the stationary phase material and carrying out flow-through chromatography and/or wherein no concentration step and/or no buffer exchange step and/or no host cell protein removal step occurs in between. In some embodiments, no concentration step occurs between the step said step (c), or (d), or (e), and flow-through chromatography step (i). In some embodiments, no buffer exchange step occurs between the step said step (c), or (d), or (e) and flow-through chromatography step (i). In some embodiments, no host cell protein removal step occurs between the step said step (c), or (d), or (e), and flow-through chromatography step (i).

As outlined above, operating the flow-through chromatography in flow-through mode advantageously allows a high recovery of MV particles. Furthermore, impurities, preferably host cell proteins and/or nucleic acids can be significantly reduced to a low level. Hence, in a further embodiment of the present invention, there is provided a method according to the first aspect of the invention and the various embodiments thereof, wherein the MV titer in the flow-through is at least 20%, preferably at least 30%, more preferably at least 40% and most preferably at least 50%, or even at least 70%, preferably at least 80% and most preferably at least 90%, of the MV titer in the sample loaded onto the flow-through chromatography unit; and/or wherein the host cell protein content in the flow-through is 60% or less, preferably 50% or less, more preferably 40% or less and most preferably 30% or less, or even 25% or less and preferably 20% or less, relative to the host cell protein content in the sample loaded onto the flow-through chromatography unit; and/or wherein the polynucleotide content in the flow-through is 70% or less, preferably 60% or less, more preferably 50% or less and most preferably 60% or less, relative to the polynucleotide content in the sample loaded onto the flow-through chromatography unit.

The finding that column chromatography, in particular packed bed column chromatography, is suited for the purification of MV particles was unexpected in view of the difficulties encountered when working with MV, which difficulties arises from the fact that MV is huge and fragile. In accordance with this finding, in a further embodiment of the present invention, there is provided a method according to the first aspect of the invention and the various embodiments thereof, wherein the flow-through chromatography step involves column chromatography. The stationary phase material is preferably selected from the group consisting of at least a resin, at least a matrix, at least a gel and, preferably, beads.

It is further preferred that the stationary phase material, preferably the beads, has/have at least size-exclusion functionality, hydrophobic interaction functionality or ion exchange functionality, or a combination thereof, preferably at least size-exclusion functionality, optionally combined with hydrophobic interaction functionality. Preferred beads may be core beads. These beads do not carry ligands at the outside/surface of the beads, but only in the inside. Therefore, substances not small enough to enter the core, will not be able to interact in a specific manner with the ligands. In contrast to gel filtration or size exclusion, where the separation is exclusively based on the size of the substances to be purified (which can only be directed by the flow rate), core beads in general have the advantage that higher flow rates can be used (small substances can easily enter the cores with the specific ligand, large molecules will rapidly be eluted without clogging the entry into the core for small substances). Another advantage of core beads in general (independent of the specific ligand used in the core) is the fact that much higher loading volumes can be used in comparison to size-exclusion/gelfiltration formats, where only a very small percentage of the actual column volume can be loaded onto the column as sample to be purified. Therefore, a stationary phase material in general should have an inert surface and very small pores to be suitable for a flow through approach according to the present disclosure.

With respect to the physical structure, materials with extremely large pores may be suitable for certain embodiments so that the MV can pass and bind, as it is the case for membrane adsorber, monoliths or fibers, but a process will specifically have to be configured for each new material. Notably, conventional chromatographic beads will suffer the huge disadvantage that the largest pores presently available (macroporous resins) have a pore size of around 400 nm. This will not suffice to allow entry of a MV during column purification simply as the MV or MV structure cannot diffuse into the beads.

The bind-elute mode in contrast to the flow-through mode) also requires that a bound structure, e.g., a virus, cannot only be bound tightly and specifically, but it can also be eluted later on under physiological conditions. This often represents a huge problem in bind-elute settings. In particular, the elution causes huge problems for MV, as it is a large virus having many binding sites making strong interactions with the material. Harsh elution conditions, however, will influence the yield of intact MV. Furthermore, the elution can result in pressure problems, as the increased concentration results in a higher viscosity and thus in a higher shear stress when leaving the flow rate constant.

All these factors might explain why certain membrane adsorber materials, monolithic materials and Eshmuno gels (see below in the Examples section) can likely cause problems when purifying large and pleomorphic MV in high yields and simultaneously with high purities and economically favorable high flow rates in a flow-through mode without further adaptions.

In a further embodiment of the present invention, there is provided a method according to the first aspect of the invention and the various embodiments thereof, wherein the stationary phase material, preferably the beads, comprises/comprise a ligand-activated core, and an inactive shell comprising pores. The pores comprised in the inactive shell have a molecular weight cut off smaller than the MV particles to exclude the MV particles from entering the ligand-activated core, whereas a molecule smaller than the molecular weight cut off can enter the pores and bind to the ligand-activated core. Preferably, the ligand-activated core comprises octylamine. With regard to the pores, it is preferred that the molecular weight cut off is in the range of 100 kDa to 2,000 kDa, preferably 200 kDa to 1,500 kDa, more preferably 400 kDa to 1,200 kDa and most preferably 500 kDa to 1,000 kDa.

According to a specifically preferred embodiment, the flow-through chromatography step (i) involves a column chromatography using Capto Core 700 stationary phase material. Capto Core 700 is composed of a ligand-activated core and inactive shell. The inactive shell excludes large molecules (cut-off 700 kDa) from entering the core through the pores of the shell. These larger molecules are collected in the column flow-through while smaller impurities bind to the internalized ligands. The core bead technology and multimodal, octylamine ligand give Capto Core 700 dual functionality, namely size exclusion and binding properties. Notably, further cut-off ranges may be suitable for certain applications according to the present invention, wherein smaller cut-offs can be associated with a higher degree of impurities, whereas large cut-off ranges might complicate resin production.

The MV particle-containing flow-through can be directly used as a feed for filtration step (ii). Thus, in one embodiment of the present invention, there is provided a method according to the first aspect of the invention and the various embodiments thereof, wherein the flow-through is directly used as a feed for the filtration. In this case, there is no additional step arranged between flow-through chromatography step (i) and filtration step (ii). In another embodiment of the present invention, there is a further chromatographic step arranged between flow-through chromatography step (i) and filtration step (ii). In this case, the MV particles containing eluate of said further chromatographic step is directly used as a feed for the filtration. Regardless whether there is an additional step between flow-through chromatography step (i) and filtration step (ii), due to the inherent difficulties to establish efficient chromatography techniques for the purification of MV particles, the method of the invention preferably comprises the flow-through chromatography step of (i) as the only chromatography step in order to maximize yield (recovery).

In a preferred embodiment of the present invention, there is provided a method according to the first aspect of the invention and the various embodiments thereof, wherein simultaneously with or sequentially to the filtration the retentate buffer is exchanged. In other words, the retentate buffer in which the MV particles are contained is exchanged by adding a (fresh) buffer to the feed reservoir as the filtration, preferably ultrafiltration, progresses in a process called diafiltration.

In case filtration and buffer exchange occur simultaneously, this may mean that the retentate volume or mass is kept at a constant level by adding exchange buffer. In case filtration and buffer exchange occur sequentially, this may mean that the retentate volume or weight is reduced, e.g., to 10% of the initial level, in a first step by (ultra-) filtration and, in a second step, exchange buffer is added at the same rate as permeate forms. This can be controlled by maintaining a constant retentate volume or weight. In some embodiments, the step of retentate buffer exchange comprises adding a solution comprising sorbitol, wherein the solution preferably comprises sorbitol in an amount of 1-20 weight-%, more preferably 2-15 weight-%, even more preferably 3-10 weight-% and most preferably 5±2 weight-%. In some embodiments, the filtration and, if present, the buffer exchange is carried out using a filter comprising regenerated cellulose. Advantageously, such filters do not show significant binding to MV particles. In some embodiment, the filters have a Molecular Weight Cut-Off (MWCO) of 10 to 1,000 kDa, preferably 15 to 800 kDa, more preferably 20 to 500 kDa and most preferably 30 to 300 kDa.

Because MV particles tend to adsorb to hollow fiber module, filtration step (ii), i.e. virus concentration, and if present the buffer exchange step, preferably does not involve hollow fiber filtration. Instead, in a preferred embodiment, the filtration step (ii), and if present the buffer exchange step, involves an ultrafiltration (UF) membrane and/or an UF cassette. Suitable membranes comprise cellulose, in particular regenerated cellulose (RC) or stabilized cellulose (SC), highly cross-linked regenerated cellulose, polysulfone (PS), modified polyethersulfone (mPES), polyvinylidene fluoride (PVDF) or a combination thereof. In preferred embodiments, the UF membrane comprises RC or modified RC, more preferably a composite RC and most preferably a RC membrane casted onto a microporous polyethylene substrate. RC and modified RC have shown the best trade-off between unspecific binding to MV particles, mechanical strength, and resistance to cleaning procedures (chemical agents and temperature).

More specifically, ultrafiltration/diafiltration was operated by ÄKTA flux s (GE Healthcare, Uppsala, Sweden). 300 kDa composite regenerated cellulose (RC) cassettes (Pellicon® XL, Merck KGaA, Darmstadt, Germany), 300 kDa polysulfone hollow fiber (HF) modules (Start AXM Cartridge, GE Healthcare, Uppsala, Sweden) and 30 kDa and 100 kDa stabilized cellulose (SC) based membranes (Hydrosart®, Sartorius, Gottingen, Germany) were tested. A membrane area of 50 cm² was tested and the membrane feed was kept constant at 110 L/m². The transmembrane pressure was kept constant throughout the process between 0.4 to 0.5 bar by adjusting a retentate pressure control valve. Membranes were pre-treated and flushed as described in the particular manufacturer's instructions before the experiments. Feed material was chromatographically pre-purified using Capto Core 700 and frozen material was thawed at 37° C. Throughout the filtration experiments samples were collected from the feed material (F), the concentrate (C) and the permeate (P) at stored at −80° C.

The MWCO of the UF membrane and/or cassettes preferably ranges between 5 and 1,000 kDa, preferably 10 to 700 kDa, more preferably 15 to 500 kDa, even more preferably 20 to 400 kDa, even more preferably 30 to 300 kDa, and most preferably 100 to 250 kDa. Furthermore, in particular preferred embodiments, the UF membrane and/or cassettes are characterized by a MWCO of 300 kDa, in particular if a RC based UF membrane is used. In other preferred embodiments, the UF membrane and/or cassettes are characterized by a nominal MWCO of 30 kDa, in particular if a SC based UF membrane is used. In a preferred embodiment, the UF membrane and/or cassettes are characterized by a nominal MWCO range from about 30 to 150 kDa. For example, a UF membrane with a MWCO of around 100 kDa may allow the possibility of a stronger concentration and a better purity for certain MV preparations. Having, for instance, a MV preparation with relatively large virus particles (on average), a 100 kDa UF membrane may be preferred over a 30 kDa membrane, for instance, as this may result in a 50- to 100-fold concentration in contrast to a 10-fold concentration when using a 30 kDa membrane for the same preparation. An optimum nominal MWCO for the UF membrane and/or cassettes can thus be determined based on the above by pre-testing. Obtaining a highly concentrated intermediate MV-product after this UF membrane step significantly simplifies the formulation of a final drug product. Furthermore, a higher concentration allows for higher concentrations in case a high MV dose is needed for certain MV applications.

In a yet further embodiment, the filtration step (ii), and optionally the buffer exchange step, is carried out by means of at least two membranes and/or at least two cassettes, having different MWCO, e.g. one having 100 to 1,000 kDa and/or another having 10 to 100 kDa, preferably one having 150 to 700 kDa and/or another having 15 to 70 kDa, more preferably one having 200 to 500 kDa and/or another having 20 to 50 kDa and most preferably one having 100 to 300 kDa and/or another having 30 kDa. This different MWCO choice may allow to compensate for the fact that MV is strongly pleomorphic and thus allows a better and more homogeneous purification with simultaneously relatively high yields (low losses during filtration).

In a further embodiment of the present invention, there is provided a method according to the first aspect of the invention and the various embodiments thereof, wherein the filtration and, if present, the retentate buffer exchange involves tangential flow filtration. The tangential flow filtration is preferably carried out at a transmembrane pressure in the range of 0.1 to 1 bar, preferably 0.2 to 0.8 bar, more preferably 0.3 to 0.6 bar and most preferably 0.4 to 0.5 bar. It is further preferred that the transmembrane pressure is maintained within said range throughout the filtration and, if present, the retentate buffer exchange.

It is to be understood that the various embodiments of the present invention can be used to purify VLPs based on MV. VLPs lack genetic information and are thus non-replicative. VLPs per se are thus non-infectious in the sense that they cannot replicate in a cell to give rise to new viral particles and thus to spread to further cells after a replicative cycle. Still, VLPs, after their assembly and based on the molecules exposed on their surface, can interact with a host cell, e.g. through surface receptors, or, after uptake and/or processing by an immune cell, e.g. an antigen-representing cell, epitopes or antigens comprised by a VLP can be presented or cross-presented by the immune cell to effector cells. By means of this interaction, VLPs can induce an immune response in an organism. This ability makes VLPs suitable structures for the provision of safe immunogenic or vaccine compositions. Hence, in further embodiments, the MV particles comprise, or essentially consist of, VLPs derived from MV. In case the sample to be subjected to flow-through chromatography step (i) comprises both virions and VLPs, the method may additionally comprise a further purification step preceding or following flow-through chromatography step (i), comprising: further purifying the virus particles comprised in the sample, respectively flow-through by means of at least one separation technique selected from the group consisting of filtration, centrifugation, tangential flow filtration, membrane filtration, purification with grafted media, aqueous two phase extraction, precipitation, buffer exchange, dialysis or chromatography, including size exclusion chromatography, e.g. for separating the virus particles into a fraction containing virions and another fraction containing virus-like particles (VLPs) (VLPs). VLPs possess relevant surface antigens, but cannot further be propagated in a host cell. This makes VLPs an interesting target for several applications in immunology.

In a further embodiment of the present invention, there is provided a method according to the first aspect of the invention and the various embodiments thereof, wherein the MV particles are selected from the group consisting of live, attenuated and inactivated virus particles, or a mixture thereof, and/or wherein the MV particles are recombinant and/or infectious particles, preferably infectious recombinant particles.

According to all aspects and embodiments of the present disclosure, the MV particles are preferably derived from an attenuated virus strain, preferably being selected from the group consisting of the Schwarz strain, the Zagreb strain, the AIK-C strain and the Moraten strain. In a particular preferred embodiment, a MV particle is encoded by a nucleic acid molecule comprising all or parts of the antigenomic region of a MV, preferably including further recombinant enhancements. A suitable MV scaffold is the MV Schwarz strain pTM having a nucleic acid sequence as shown in SEQ ID NO:1. Further suitable MV scaffolds with ATUs as backbone structures are represented in SEQ ID NOs:2 and 3. The skilled person is well aware of the fact that a sequence having slightly varying deviations from the exemplary sequences presented herein, e.g., a sequence identity of at least 75%, 80%, 85%, 90%, 91%, 92%, 93%, 94%, 95%, 96%, 97%, 98%, or 99% (or even less, e.g., in the case that a sequence is codon optimized for use in a specific host cell) may still be suitable for the purpose of the present invention as long as there is no mutation at a position encoding an essential amino acid or the like, or that no stop codon is introduced, as long as the encoded sequence still encodes all relevant features of a MV-based construct and optionally for an insert of interest.

Moreover, the MV particles herein may comprise (exogenous) genetic material for expressing one or more (foreign) antigens. As such, a platform is provided that is based on the well-established MV vector in order to expose a subject to any antigen of interest. This design enables any antigen to be generated in vivo in infected cells, in particular in infected cells of a mammalian, and thus to elicit an immune response and/or provide efficient and long-lasting immunity, especially which induces life-long immunity after only a single or two administration steps. Accordingly, in preferred embodiments of the various aspects of the present invention, the MV particles comprise, or essentially consist of, recombinant and preferably infectious virus particles derived from and/or comprising a MV scaffold, the scaffold being encoded by at least one nucleic acid sequence, wherein the nucleic acid sequence comprises a first nucleic acid sequence encoding a MV scaffold and at least one second nucleic acid sequence operably linked to the first nucleic acid sequence. The at least one second nucleic acid sequence may encode an antigen, preferably at least one foreign virus antigen, in particular if the virus particles are intended to be used in a vaccine, in particular against infection with said virus(es), which naturally comprise(s) said at least one foreign virus antigen. The at least one second nucleic acid sequence may also encode a protein or a regulatory RNA other than a viral antigen, for example a micro RNA (miRNA), assisting tumor treatment, preferably in a mammalian, or more particularly in a human subject, in particular in case the MV particles are for use in OV therapy. The protein or RNA encoded by the second nucleic acid may for example mediate the interaction between a MV particle and a tumor cell and/or its uptake into a tumor cell. In further embodiments, the second nucleic acid sequence may encode a gene toxic for a tumor cell of interest, e.g. a suicide gene comprising a fusion of a cytosine deaminase, particularly yeast cytosine deaminase, and an uracil phosphoribosyltransferase, particularly yeast uracil phosphoribosyltransferase. In yet further embodiments, the second nucleic acid sequence encodes a protein/RNA that enhances antitumor cytotoxicity and immunity. Furthermore, the second non-viral nucleic acid can be configured to promote a strong anti-tumor immune response, e.g. by activating antigen presenting cells, preferably dendritic cells, for example plasmacytoid dendritic cells, by activating their ability to produce high quantities of IFN-α and/or to cross-present tumor antigens from infected tumor cells to tumor-specific CD8+T lymphocytes to achieve a strong cellular immune response against the tumor cells or tissue. “Cross-presentation” or “cross-presenting” in this context means the ability of certain antigen-presenting cells to take up, process and present extracellular antigens with MHC class I molecules to CD8+ T cells (cytotoxic T cells). Cross-priming, the result of this process, describes the stimulation of the naïve cytotoxic CD8+ T cell. This process is necessary for immunity against most tumors and against viruses that do not readily infect antigen-presenting cells or impair dendritic cell normal function. It is also required for induction of cytotoxic immunity by vaccination with protein antigens, for example, tumor vaccination.

In accordance with this embodiment, the nucleic acid construct encoding a recombinant infectious virus particles comprising an infectious MV (MV) scaffold thus comprises the following gene transcription units encompassing from 5′ to 3′: (a) a polynucleotide encoding the N protein of a MV, (b) a polynucleotide encoding the P protein of a MV, (c) the polynucleotide encoding at least one structural protein used as antigen, for example at least one Chikungunya structural protein, suitable as antigen (d) a polynucleotide encoding the M protein of a MV, (e) a polynucleotide encoding the F protein of a MV, (f) a polynucleotide encoding the H protein of a MV, and (g) a polynucleotide encoding the L protein of a MV, said polynucleotides and nucleic acid construct being operably linked and under the control of viral replication and transcription regulatory sequences such as MV leader and trailer sequences. The expressions “N protein”, “P protein”, “M protein”, “F protein”, “H protein” and “L protein” refer respectively to the nucleoprotein (N), the phosphoprotein (P), the matrix protein (M), the fusion protein (F), the hemagglutinin protein (H) and the RNA polymerase large protein (L) of a MV. The expression “operably linked” refers to the functional link existing between the at least one antigen encoding nucleic acid sequence according to the methods of the invention such that said at least one nucleic acid sequence within the MV scaffold is efficiently transcribed and translated, in particular in cells or cell lines, especially in cells or cell lines used as cell bank according to the present invention so that an antigenic epitope can be presented after.

The nucleic acid sequence encoding at least one antigen is preferably selected from the group consisting of a nucleic acid sequence derived from a virus belonging to the family of Flaviviridae, including a nucleic acid sequence derived from a West-Nile virus (cf. NCBI reference sequence NC_009942.1), a tick-borne encephalitis virus (NCBI reference sequence NC_001672.1), a Japanese encephalitis virus (NCBI reference sequence NC_001437.1), a yellow fever virus (NCBI reference sequence NC_002031.1), a Zika virus (NCBI reference sequence NC_012532.1), or a Dengue virus (e.g. NCBI Dengue virus 1/strain Nauru/West Pac/1974: NC_001477.1), a Chikungunya virus, a norovirus (e.g. Norwalk virus, NCBI NC_001959.2), a virus belonging to the family of Paramyxoviridae, including a nucleic acid sequence derived from a human respiratory syncytical virus (RSV) (e.g. NCBI: NC_001781.1), a MV or a metapneumovirus (e.g. human: NCBI Gene ID: 2830349; avian: NCBI Gene ID: 5130032), a parvovirus (e.g. human parvovirus B19, NCBI: NC_001348.1), a coronavirus, including a nucleic acid sequence derived from a Middle East respiratory syndrome antigen (see e.g. NCBI: NC_019843.3), or a severe acute respiratory syndrome antigen (e.g. NCBI: NC_004718.3), a human enterovirus 71 (e.g. enterovirus A, NCBI: NC_001612.1), a cytomegalovirus (e.g. human herpesvirus 5, NCBI: NC_006273.2), a poliovirus (e.g. human enterovirus C serotype PV-1, NCBI: NC_002058.3, an Epstein-Barr virus (e.g. human herpesvirus 4, NCBI: NC_009334.1 or NC_007605.1, respectively), a hepatitis E virus (e.g. NCBI: NC_001434.1), a human papilloma virus, preferably a human papilloma virus 16, a human papilloma virus 5, a human papilloma virus 4, a human papilloma virus 1 or a human papilloma virus 41 (see e.g. NCBI: HPV-16: NC_001526.2; HPV-5: NC_001531.1; HPV-4: NC_001457.1; HPV-1: NC_001356.1; HPV-41: NC_001354.1), or a varicella zoster virus (e.g. human herpesvirus 3, NCBI: NC_001348.1).

Further details of suitable nucleic acid constructs encoding MV particles are for example disclosed in EP 2 712 871 A1 and EP 3 184 119 A1, which are herein incorporated by reference, in particular for the purpose of preparation and design of nucleic acid constructs comprising genetic information of MV (scaffold) and one more foreign antigens, vectors comprising said nucleic acid constructs as well as the resulting recombinant infectious (replicating) MV particles prepared from said vectors.

From a structural point of view, the MV scaffold represents the majority of material to be transcribed/translated into a virus particle. As the purification and/or production method according to the various embodiments according to the first aspect of the present invention has been specifically designed to produce and/or purify MV particles by relying on physico-chemical properties of the MV scaffold comprised as structural element by the MV particles, any foreign antigen and in particular any of the above antigens operably linked to the MV scaffold can thus be provided by the present invention in high yields and high purity. Hence, also virus particles derived from a MV scaffold comprising other antigens than those disclosed herein can be purified according to the methods of the present invention. The broad applicability of the method according to the present invention are elucidated by the fact that despite different antigens can be expressed, as a common feature the same or similar scaffold structure needs to be produced and/or purified.

Further examples for a nucleic acid sequence encoding a MV particle according to the disclosure of the present invention, which can be produced and/or purified according to the methods of the present invention, comprise a nucleic acid sequence according to SEQ ID NO:4 (MV GFP or MV-GFP with green fluorescent protein encoding insert), SEQ ID NO:5 (MV-Chikungunya, MV-CHIK), SEQ ID NO:6 (MV-Dengue or MV DVAX1) or SEQ ID NO:7 (MV Zika sE (soluble E-protein)) or, or a homologous sequence having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto provided that the homologous sequence after transcription and optionally translation in a cell population or host cell still results in a MV scaffold optionally operably linked to at least one antigen of at least one virus, which is infectious and/or immunogenic, but does not comprise a relevant mutation in a region of the MV genome, which would disturb its natural replication cycle, or which would revert the attenuated MV scaffold back into a non-attenuated virus form. Said sequence homology range is thus caused by the fact that a MV scaffold can, by means of recombinant technology, comprise codon optimized positions, further regulatory or antigen positions and the like. Said modifications would, however, not significantly alter the physical and chemical properties of the MV particle, which properties are responsible for production and/or purification by the method of the invention.

Moreover, the MV particles may alternatively, or additionally to the aforementioned antigens, comprise (exogenous) genetic material for expressing one or more (foreign) proteins. Suitable genetic material may include marker genes, whose expression can be easily determined, e.g. via detection of electromagnetic radiation. Therefore, the at least one second nucleic acid sequence operably linked to the first nucleic acid sequence may encode a protein having properties that allow its easy determination, e.g. via detection of electromagnetic radiation, and/or being capable of emitting electromagnetic radiation. In particularly preferred embodiments of the present invention, the at least one second nucleic acid sequence encodes for a fluorescent protein such as Green Fluorescence Protein (GFP). An exemplary nucleic acid sequence encoding for the corresponding MV particle (MV GFP) is shown in SEQ ID NO:4.

Furthermore, according to any aspect, and any embodiment thereof, of the present invention, the disclosed nucleic acid molecules can be further modified by means of molecular biology to introduce a new or a modified regulatory sequence, restriction enzyme binding/cutting site as well as various nucleic acid sequences encoding an antigenic region of interest, preferably respecting the above identified “rule of six”. This rule was established for certain viruses belonging to the Paramyxoviridae family where the MV scaffold of the present disclosure phylogenetically is derived from/belongs to. This rule is thus derived from the fact that in order for the entire process of RNA synthesis, genome replication and encapsidation which the MV proceeds through in a host cell to be efficient at generating full-length genomic and antigenomic molecules it is necessary that the viral genome is enclosed within its protein coat, specifically the N proteins. Without this, the virus replication machinery will find problems to begin replication. Each N molecule associates with exactly 6 nucleotides, which explains the reason as to why these viruses require their genomes to be a multiple of six. It is thus evident that a variety of modifications of the MV scaffold can be undertaken with the proviso that it still results in a MV scaffold able to infect a host cell. Therefore, means like codon optimization and the like can be applied as long as no mutation introduced which would change the functional properties of a regulatory sequence or a structural protein of the MV. Codon optimization implies that the codon usage of a given nucleic acid sequence can be modified to be compatible with the codon usage of a host cell of interest to allow better transcription rates and the expression of functional amino acid sequences in a host cell of interest. Furthermore, in the case of virions comprising genetic material, it has to be ensured by sequencing that the resulting purified recombinant infectious virus particles derived from a MV scaffold do not comprise a mutation rendering the attenuated virus virulent again. Such methods of nucleic acid sequencing, including deep sequencing, for means of sequence confirmation belong to the common general knowledge of the skilled person in the field of molecular biology and virology and can be applied at any stage of the methods according to the present disclosure.

As outlined above, any recombinant virus particle comprising and/or derived from a MV scaffold, can be purified according to the methods of the present invention, as the methods are specifically optimized taking into consideration the peculiar chemical and physical properties of the huge and pleomorphic MV, which properties are mainly influenced by the MV envelope/capsid making up the majority of the surface accessible area of the recombinant virus particle comprising and/or derived from a MV scaffold. A skilled reader will further note from the present disclosure that the purification steps comprised in the method of the invention rely mainly on the huge size of the MV particles as compared to the small size of the one or more impurities, e.g. host cell proteins and/or nucleic acids. It is therefore evident that the method of the present invention is also particularly useful for the production and/or purification of other huge and sterically-demanding virus particles, in particular those having a size similar to MV, e.g., having a size in the range of 50 nm and 2 μm, preferably in the range of 80 nm and 1.5 μm, more preferably in the range of 100 nm and 1,000 nm, even more preferably in the range of 100 nm and 500 nm. The present disclosure is not restricted to the purification of Paramyxoviridae or MV particles but encompasses methods according to the various embodiments of the first aspect of the invention, wherein instead of MV particles other sterically-demanding virus particles, preferably having a viral envelop covering the capsid, and in particular those described above, are produced and/or purified. Specific classes of enveloped viruses containing human pathogens, which can be purified according to the present disclosure, comprise, for example, Herpesvirus, Poxvirus, Hepadnavirus, Flavivirus, Togavirus, Coronavirus, Hepatitis D virus, Orthomyxovirus, Rhabdovirus, Bunyavirus, Filovirus, or certain retroviruses.

In a particular embodiment of the methods of the present invention, the construct is prepared by cloning a polynucleotide sequence encoding one structural protein or a plurality of structural proteins of a virus other than a MV in the cDNA encoding the full-length antigenomic (+) RNA of the MV. Alternatively, a nucleic acid construct of the disclosure may be prepared using steps of synthesis of nucleic acid fragments or polymerization from a template, including by PCR. It is further disclosed that the polynucleotide encoding the at least one protein of the virus other than a MV, or each of these polynucleotides, is cloned into an ATU (Additional Transcription Unit) inserted in the cDNA of the MV. Usually, there is one ATU per construct. ATU sequences usually comprise three potential regions of inserting a nucleic acid and further comprise, for use in steps of cloning into cDNA of MV, cis-acting sequences necessary for MV-dependent expression of a recombinant transgene, such as a promoter preceding a gene of interest, in MV cDNA, the insert represented by the polynucleotide encoding the viral protein(s) inserted into a multiple cloning sites cassette. The ATU is advantageously located in the N-terminal sequence of the cDNA molecule encoding the full-length (+)RNA strand of the antigenome of the MV and is especially located between the P and M genes of this virus or between the H and L genes. It has been observed that the transcription of the viral RNA of MV follows a gradient from the 5′ to the 3′ end. This explains that, when inserted in the 5′ end of the coding sequence of the cDNA, the ATU will enable a more efficient expression of the heterologous recombinant nucleic acid DNA sequence. The ATU sequence can, however, be located at any position of SEQ ID NO:1 provided that it does not disrupt a coding sequence or a regulatory sequence thereof.

As outlined above, the method of the various embodiments of the present invention can advantageously be used to produce and/or purify recombinant infectious virus particles for providing said particles as vaccines or the like. A huge advantage of such produced and/or purified replication-competent vector, i.e. the recombinant infectious virus particles, present in a therapeutic or prophylactic composition is that it may provide a powerful, antigen-focused immune response to confer long-term immunity by continuously expressing antigens even after immunization.

In a second aspect of the present invention, there is provided a system for producing and/or purifying MV particles. The system comprises or consists of: (i) at least one bioreactor; (ii) a clarification unit, preferably a dead-end filter unit, downstream to the bioreactor; (iii) a flow through chromatography unit downstream to the clarification unit; and (iv) a filtration unit, preferably a tangential flow filtration unit, downstream to the flow through chromatography unit.

The term “consisting of” in the context of a system for producing and/or purifying MV particles is to be understood to refer to a series of operation units consisting of (i) at least one bioreactor; (ii) a clarification unit, preferably a dead-end filter unit, downstream to the bioreactor; (iii) a flow through chromatography unit downstream to the clarification unit; and (iv) a filtration unit, preferably a tangential flow filtration unit, downstream to the flow through chromatography unit. In this case, no further purification unit(s), concentration unit(s) and/or buffer exchange unit(s), and preferably no operation unit(s) at all (besides the optional nucleic acid digestion unit), is arranged between the at least one bioreactor and the filtration unit. Other operation units may optionally be arranged upstream of the at least one bioreactor and/or downstream of the filtration unit, in particular operation units for filling and/or packaging and the like. In some embodiments, the whole system does not comprise any further purification unit, concentration unit and/or buffer exchange unit. In other words, the aforementioned series of operation units may make up a whole process or form a consecutive part of an overall bigger process. Tanks such as surge tanks are not to be understood as operation units in the context of the present invention. As such, a system consisting of operation units may comprise surge vessels between some or all of the operation units. Moreover, since nucleic acid digestion is usually carried out in a tank, a system consisting of the above operation units may optionally comprise a nucleic acid digestion unit that follows the last of the least one bioreactor or the clarification unit.

Herein, a bioreactor is an apparatus, which allows cultivation, in particular cell growth, of microorganisms, preferably a pure culture, under appropriate conditions suitable for cell growth. Usually, a sterile bioreactor is inoculated with microorganisms to be grown under aseptic conditions. To maintain a pure culture over prolonged time, a bioreactor is to be understood to be designed so that contamination with other living organisms prevented. To provide a surface on which the host cells can efficiently grow, the at least one bioreactor preferably includes microparticles such as those described in the context of the first aspect of the invention. Moreover, the bioreactor may be a filled bioreactor including at least one host cell and/or MV particles.

A clarification unit herein denotes an operation unit suitable for carrying out clarification, i.e. a step for removing large impurities from a bulk product such as a cell culture supernatant to be clarified. It shall not be understood to optionally comprise an operation unit different to the clarification unit. In the present context, the clarification unit is suitable for removing cells and cell debris from the host cells infected with and producing MV particles. Suitable means for clarification include centrifugation, ultracentrifugation, microfiltration, including tangential flow filtration, dead end filtering and the like. In preferred embodiments, the clarification unit does not rely on separation by density, e.g. does not comprise a centrifuge. As outlined in the context of the first aspect of the present invention, the clarification unit preferably comprises a dead-end filter, preferably one or more depth filters. In order to be suited for clarification the dead-end filter must allow MV particles to pass through the pores of the filter, while large impurities such as cells and cell debris are retained. Further preferred embodiments in relation to the clarification unit comprised in the second aspect of the present invention are described in the context of clarification step (d) according to an embodiment of the first aspect the invention.

The flow-through chromatography unit as defined herein at least comprises a stationary phase material that is preferably selected from the group consisting of at least a resin, at least a matrix, at least a gel and, preferably, beads. In a preferred embodiment of the second aspect of the present invention, the stationary phase material is comprised in a container, e.g. for carrying out column chromatography. In preferred embodiments, the stationary phase material, preferably the beads, has/have at least size-exclusion functionality, hydrophobic interaction functionality or ion exchange functionality, or a combination thereof, preferably at least size-exclusion functionality, optionally combined with hydrophobic interaction functionality. Further preferred embodiments in relation to the flow-through chromatography unit, and in particular in relation to the stationary phase material, comprised in embodiments of the second aspect of the present invention are described in the context of flow-through chromatography step (i) comprised in embodiments of the method according to the first aspect the invention.

For example, in preferred embodiments of the second aspect of the present invention, the stationary phase material, preferably the beads, comprises/comprise a ligand-activated core, and an inactive shell comprising pores. The pores comprised in the inactive shell have a molecular weight cut off smaller than the MV particles to exclude the MV particles from entering the ligand-activated core, whereas a molecule smaller than the molecular weight cut off can enter the pores and bind to the ligand-activated core. Preferably, the ligand-activated core comprises octylamine. With regard to the pores, it is preferred that the molecular weight cut off is in the range of 100 kDa to 2000 kDa, preferably 200 kDa to 1500 kDa, more preferably 400 kDa to 1200 kDa and most preferably 500 kDa to 1,000 kDa. According to a specifically preferred embodiment, the flow-through chromatography unit comprises Capto Core 700 stationary phase material contained in a container such as a column.

The filtration unit comprised in the system of the second aspect of the invention shall be generally understood to allow an increase of a concentration of MV particles. Hence, in a preferred embodiment, the filtration unit comprises a filter for retaining MV particles. The filter may be designed for letting liquid, preferably along with further impurities, pass through the filter. In preferred embodiments, the filtration unit comprises an ultrafiltration module (preferably a TFF module), a feed tank, a retentate line connecting an outlet on a retentate side of the filtration unit with the feed tank, a feed line connecting an inlet on the retentate side of the ultrafiltration module with the feed tank and a permeate line connected to an outlet on a permeate side of the ultrafiltration module. Further tanks may be included, for example, a permeate receiving tank connected to the permeate line and/or a tank for holding diafiltration buffer connected via a diafiltration line to the feed tank, the retentate line or an inlet on the retentate side of the ultrafiltration module. Furthermore, the filtration module may comprise one or more flow generating means such as pumps, for example, at least one flow generating means in the feed line and/or at least one flow generating means in the diafiltration line. The filtration module may further comprise one or more valves such as a check valve arranged in the retentate and/or permeate line. Furthermore, at least one pressure sensor must be present to enable a constant pressure during the methods, or in the system according to the present invention. Preferably, a pressure sensor is at least present in the feed line, the retentate line and the permeate line to ensure a constant pressure and thus process control.

Further details of preferred embodiments relating to the filtration unit result from the various embodiments described in the context of the first aspect of the invention, which shall be taken to define embodiments of the second aspect of the present invention.

In one embodiment of the second aspect of the present invention, there is provided a system according to the second aspect of the invention and the various embodiments thereof, wherein the bioreactor includes a probe for determining viable cell density and/or total cell density. The viable and/or total cell density probe is preferably based on (an) on-line technique(s). As outlined in the context of an embodiment according to method of the first aspect of the invention, an exemplarily, and herein preferred, technique for on-line determination of viable cell density is based on capacitance. This allows on-line determination in real-time and is insensitive to microcarriers as well as cell debris. The total cell density determination may be based on on-line measurements including turbidity and optical density measurement at NIR (Near Infra-Red) wavelengths, or any other suitable measurement, whether allowing on-line or off-line determination of total cell density.

In correspondence of the various embodiments according to the first aspect of the invention, the system according to the second aspect of the present invention does not necessarily reflect a method that is based on a concentration step at an early step of downstream processing. For the advantages associated with such feature, reference is made to the corresponding part of the first aspect of the present invention. Hence, in one embodiment of the second aspect of the present invention, there is provided a system according to the second aspect of the invention and the various embodiments thereof, wherein no concentration unit, preferably no ultrafiltration-based concentration unit and more preferably no tangential flow filtration unit, is arranged between the at least bioreactor and the flow through chromatography unit. Further embodiments can be deduced from the description made in the context of the first aspect of the present invention, which, as repeatedly emphasized herein, shall be understood to form corresponding embodiments in the context of the second aspect of the invention.

The system according to the second aspect of the invention, and the various embodiments thereof, can be advantageously used for continuous and/or integrated processing. In one embodiment of the second aspect of the present invention, there is provided a system, wherein the clarification unit, preferably the dead end filter unit, is either in direct fluid communication with the bioreactor or via a first vessel; and/or wherein the flow through chromatography unit is either in direct fluid communication with the clarification unit, preferably the dead end filter unit, or via a second vessel; and/or wherein the filtration unit, preferably the tangential flow filtration unit, is either in direct fluid communication with the flow through chromatography unit, or via a third vessel; and preferably wherein the first vessel is a storage vessel or a mixable vessel, and/or the second vessel and/or the third vessel is a storage vessel.

A main difference between the prior art methods and systems for producing and/or purifying MV particles is that it relies on flow-through chromatography. Using a flow-through chromatography unit in a method or system according to the first, respectively second aspect of the present invention overcomes problems relating to low purity in case no chromatography step at all is used, and low recovery in case the chromatography step is performed in bind-elute mode. A hypothesis for these observations has been provided above in the context of the first aspect of the present invention. Hence, a third aspect of the present invention relates to the use of flow-through chromatography as defined in the various embodiments of the first and second aspect of the present invention for the purification of MV particles.

Due to the high purity and high recovery that can be achieved by the first and second aspect of the invention, the various embodiments of these aspects are particularly useful in providing a prophylactic and/or therapeutic composition. Hence, in a fourth aspect of the present invention, there is provided a prophylactic or therapeutic composition produced by the method according to the various embodiments of the first aspect of the invention and/or the system according to the various embodiments of the second aspect of the invention, wherein infectious MV particles are comprised in the composition as an active ingredient.

Preferred prophylactic and/or therapeutic compositions include immunogenic and/or vaccine compositions, which are suitable and/or intended to be used for use in a method of eliciting an immune response, or in a method of prophylactic treatment of a subject for protecting the subject from infection with a virus. In such method, protection is achieved by exposing the subject to the MV particles comprised by the immunogenic composition or the vaccine composition. Further preferred prophylactic and/or therapeutic compositions include compositions, which are suitable and/or intended to be used for tumor therapy such as oncolytic tumor therapy based on infectious MV particles.

Hence in a fifth aspect of the present invention, there is provided a composition according to the fourth aspect of the present invention for use in a method of therapeutic and/or prophylactic treatment. In one embodiment of this aspect, a subject is exposed to the MV particles comprised by the composition for protecting the subject from infection with a virus and/or an immune response is elicited by exposing the subject to the MV particles comprised in the composition. In another embodiment of this aspect, a subject is exposed to the MV particles comprised by the composition for tumor therapy such as oncolytic tumor therapy and/or an immune response is elicited by exposing the subject to the MV particles comprised in the composition.

In a sixth aspect of the present invention, there is provided a method of prophylactic and/or therapeutic treatment, the method comprising exposing a subject to the MV particles comprised by the composition of the fourth aspect of the present invention. In one embodiment of this aspect, the method is used for protecting the subject from infection with a virus. In another embodiment of this aspect, the method is used for (oncolytic) tumor therapy. In both cases, the method may comprise exposing the subject to the MV particles comprised in the composition and/or elicit an immune response by exposing the subject to the MV particles comprised in the composition.

In a seventh aspect, the present invention relates to use of MV particles, obtained by the various embodiments of the method according to the first aspect or the various embodiments of the system according to the second aspect of the invention, for the manufacture of a medicament for therapeutic and/or prophylactic treatment, in particular as a vaccine or in OV therapy.

The invention will now be further described with reference to the following not limiting examples.

### EXAMPLES

The following acronyms are used herein above and below: Master Cell Bank (MCB), Working Cell Bank (WCB), Phosphate Buffered Saline (PBS), Bulk Drug Substance (BDS), Drug Product, In-process Sample, Water for Injection (WFI), Unpurified Harvest (UH), Benzonase treated unpurified harvest (BUH), Purified bulk (PB), Concentrated bulk (CB), Column volume (CV), Transmembrane pressure (TMP), Upstream processing (USP), Downstream processing (DSP), Room temperature (RT), Tangential flow filtration (TFF).

Examples 9 to 20 have been carried using MV-CHIK encoded by a nucleic acid having the sequence according to SEQ ID NO:5. Example 21 employed MV-GFP encoded by a nucleic acid having the sequence according to SEQ ID NO:4. These examples shall not be understood to be limited to MV-CHIK respectively MV-GFP but shall be understood to provide support for the production and/or purification of any MV particle, and in particular those specifically disclosed herein, as well as large virus particles in general that preferably have a similar size and optionally similar physico-chemical surface characteristics as MV. Moreover, the examples are intended to provide disclosure for further preferred embodiments, wherein individual features, whether disclosed alone or in combination with further features, can be isolated and combined with further features, whether disclosed in the same example or in different examples or whether occurring in the description or the claims, to define further embodiments which are covered by the present disclosure.

Furthermore, while the following examples employed a cell bank comprising specific host cells, the present invention shall not be understood to be limited to a specific type of host cells. It is clear that any host cell susceptible to infection with MV particles will be capable to produce MV particles, which will either result in autolysis or in lysis of the cell by means of a lysis step. Hence, regardless which host cells have been used to produce a supernatant can be obtained comprising MV particles and one or more impurities such as host cell proteins and/or nucleic acids. Though the precise physico-chemical properties of impurities stemming from different host cells might slightly differ, the overall purification concept still allows to readily exploit the advantages of the present invention also for these kinds of host cells. This is in part due to the fact that a substantial part of the purification scheme is based on separation by size.

### Example 1: USP Starting Material

A master cell bank (MCB) of Vero 10-87 cells, lot No 1416.01 MCB, P #145 was produced. The MCB is contained in cryovials, each containing 1 mL of cell suspension at a concentration of 1.0×10⁷ cells/mL. The MCB should be stored in a vapor phase liquid nitrogen cryogenic tank at two different locations.

From the Vero MCB a working cell bank (WCB) with a concentration of 1.0×10⁷ cells/mL was produced and 120 cryovials were stored in a vapor phase liquid nitrogen cryogenic tank.

### Example 2: WCB Revival

One cryovial of the WCB containing 1.0×10⁷ cells in a total volume of 1 mL is removed from storage in the vapor phase of liquid nitrogen and placed in a 50 mL tube containing 25 mL of 70% ethanol. The tube is transported to a cleanroom where the cryovial is removed from the ethanol and immediately thawed in hand until the cell suspension is fully thawed. For sanitization, the vial is placed back in the tube filled with 70% ethanol and transferred into a biosafety cabinet. The cryovial is poured on a layer of tissue papers and the thawed cell suspension is pipetted dropwise to a 50 mL tube filled with 10 mL VP-SFM medium. The cell suspension is subsequently centrifuged at 300×g for 5 minutes at room temperature. The supernatant is discarded and the cell pellet is loosened by gently tapping the tube to the working area of the biosafety cabinet. The cells are then re-suspended in 10.5 mL of VP-SFM medium and 0.5 mL of the suspension are removed for cell counting. Exemplary revival parameters for the WCB are shown in Table 1 below.

TABLE 1
WCB Revival Parameters

| Stage | Parameter | Operating criteria (range) |
|---|---|---|
| WCB storage | Temperature | Vapor phase liquid |
| conditions | | nitrogen |
| WCB thaw and | Thaw temperature | In hands |
| recovery | Centrifugation | 300 × g |
| Stage 0 | Centrifugation time | 5 minutes |
| | Centrifugation temperature | Room temperature |
| | Medium used | VP-SFM |
| | Pellet resuspension | 10.5 mL |
| | media volume | |

In order to determine cell number and viability 10 μL of cell suspension is mixed with 10 μL of trypan blue (0.4% in 0.81% sodium chloride and 0.06% potassium phosphate dibasic solution) in a microcentrifuge tube and briefly vortexed. 10 μL of the mixture are withdrawn and pipetted into a counting slide. Cell count and viability is measured using an automated hemacytometer (e.g. Biorad TC20). Cell counts should be documented. Usual acceptance criteria for cell viability are 85% viability. Lower viability values are possible, yet this might guarantee optimum results. The remaining 10 mL are used for cell expansion.

### Example 3: Cell Expansion

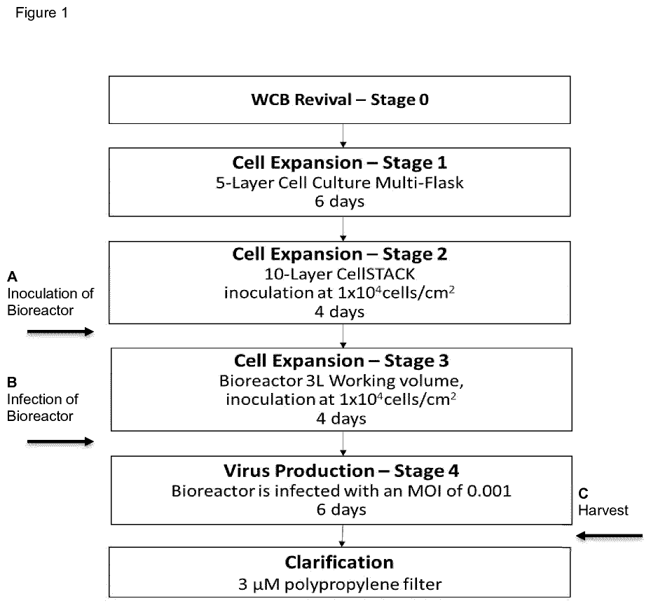

A procedure to obtain the at least one host cell and preferably the plurality of host cells is described, exemplarily for a 3-stage cell expansion using Vero 10-87 cells, in the following and illustrated in FIG. 1. Notably, the final clarification step has been performed according to different strategies, i.e., as depicted with e 3 μm polypropylene filter, but also with a filter cascade (50 μm and 3 μm).

In stage 1, the remaining 10 mL cell suspension from stage 0 are transferred to a 5-layer cell culture multi-flask filled with 140 mL VP-SFM medium and incubated for 6 days. Exemplary parameters for stage 1 are shown below in Table 2.

TABLE 2
Process parameters for stage 1

| Stage | Parameter | Operating criteria (range) |
|---|---|---|
| Cell expansion | Seeding density | From WCB Revival |
| Stage 1 | Culture volume | 150 mL |
| | Culture medium | VP-SFM |
| | Temperature | 36.5 ± 1° C. |
| | Duration | Approximately 6 ± 1 days |
| | CO₂ | 5.0% ± 1% |
| | Humidity | 80% ± 10% |
| | Final cell density | 90% confluent cells |
| | Final cell viability | 90% viability |

In stage 2, upon confluence of the 5-layer flask from stage 1, the supernatant is removed, discarded and the cell monolayer is washed with PBS. TrypLE Select is added and distributed evenly over the monolayer. The flask is incubated to detach cells and then observed for cell detachment under the microscope. If necessary, the flask is tapped gently for cell detachment. If detachment is below 90%, the flask is further incubated until detachment of greater than 90% is reached. (Parameters are the same as in Stage 3 described below).

25 mLVP-SFM medium are added to the flask and the cell suspension is transferred to a sterile centrifuge tube. The cell suspension is then centrifuged as described in Table 3 below. After centrifugation, the supernatant is removed and discarded while the cell pellet is loosened by tapping and re-suspended in 25 mL VP-SFM medium by pipetting up and down. The culture is fully suspended if no cell clumps are visible. 0.5 mL from the prepared cell suspension are removed and used to determine cell number and viability as described above.

TABLE 3
Cell harvest parameters

| Stage | Parameter | Operating criteria (range) |
|---|---|---|
| Cell harvest | PBS Cell wash volume (T875) | 50 mL |
| Stage 1 | TrypLE select volume (T875) | 25 mL |
| | Cell detachment incubation | |
| | temperature | 36.5 ± 1° C. |
| | Cell detachment | 5-10 min until 90% |
| | incubation time | detachment |
| | VP-SFM added for | 25 mL |
| | centrifugation | |
| | Centrifugation (g) | 300 × g ± 5% |
| | Centrifugation time | 5 minutes |
| | Centrifugation temperature | Room temperature |
| | Pellet resuspension media (VP- | 25 mL |
| | SFM) volume | |

Based on the determined viable cell count the cells are diluted with VP-SFM medium to a concentration of 4.2×10⁴ cells/mL in a total volume of 1500 mL and filled in a 10-layer CelISTACK. Cell expansion parameters are shown in Table 4 below:

TABLE 4
Cell expansion parameters

| Stage | Parameter | Operating criteria (range) |
|---|---|---|
| Cell expansion | Seeding density | 1.00 × 10⁴ cells/cm² |
| Stage 2 | Growth area (10-layer | |
| | CellSTACK) | 6360 cm² |
| | Culture volume (10-layer | 1500 mL |
| | CellSTACK) | |
| | Culture media | VP-SFM |
| | Incubation Temperature | 36.5 ± 1° C. |
| | Duration | Approximately 4 ± 1 days |
| | CO₂ | 5.0% ± 2% |
| | Humidity | 80% ± 10% |
| | Final cell density | 90% confluent cells |
| | Final cell viability | 90% viability |

Before starting with stage 3 of the cell expansion, the bioreactor may be prepared according to the following exemplary procedure. All bioreactor tubings and pipes are pre-assembled and the pH, DO and cell density probes are calibrated. For a process run with 3 L working volume around 9 g (or more, i.e., 18 g) dry Cytodex I microcarriers are weight in a class container and swollen in 500 mL (or more, e.g., 800 mL for 18 g microcarrier) Ca2+ and Mg2+ free PBS for at least 3 h at room temperature. The supernatant is decanted and the microcarriers are washed for a few minutes in 400 mL fresh Ca2+ and Mg2+ free PBS. The PBS is discarded and replaced with 2000 mL fresh Ca2+ and Mg2+ free PBS. The swollen microcarrier are finally transferred into the pre-assembled bioreactor vessel, the system is fully assembled and autoclaved for 20 minutes at ≥121° C.

When the bioreactor is sterilized and connected to the control unit, the microcarrier were let to settle down and the PBS is removed using overpressure. In order to wash the microcarrier 500 mL VP-SFM medium are pumped in the bioreactor vessel using a peristaltic pump and stirred at 40 rpm for 5 minutes. The microcarrier were again let to settle down and the medium is removed using overpressure. The bioreactor vessel is now filled to 2900 mL final working volume with VP-SFM using a peristaltic pump. Subsequently the heating, the stirring, all relevant process loops and the cell density probe are started and the system is let to stabilize overnight (Process parameters are shown in Table 5). The bioreactor is now ready for inoculation with the cells.

TABLE 5
Process parameters-Stage 3

| Stage | Parameter | Operating criteria (range) |
|---|---|---|
| Process | Vessel type | 3.8 L Bioreactor |
| parameters Stage | Surface area per 9 g Cytodex I | 39600 cm2 |
| 3 | Total culture volume | 2900 mL |
| | Revolutions per minute | 40 |
| | Culture media | VP-SFM |
| | Incubation Temperature | 36.5 ± 1° C. |
| | Dissolved oxygen | control by process air |
| | via sparger | >40% |
| | pH control | 7.2 ± 0.2 |
| | by CO2 via overlay (to decrease pH) and | |
| | by sodium bicarbonate (to increase pH) | |
| | | 36.5 ± 1° C. (during cell |
| | | culture) |
| | Temperature control | |
| | | 32.5 ± 1° C. (during virus |
| | | production) |
| | | fmes: 382 kHz fhigh: |
| | Cell Density probe-lncyte | 10000 kHz integration: |
| | | High |
| | Agitation control for homogeneous mixing | One pitched blade impeller |
| | Process Air | Air via overlay, 0.8 mL/min |
| | CO₂ | CO2 via overlay with pH |
| | | control |
| | Air (O₂) | Air (O₂) via sparger with |
| | | DO control |

Upon confluence of the 10-layer CelISTACK from stage 2, the supernatant is removed, discarded and the cell monolayer is washed with PBS. TrypLE Select is added and distributed evenly over the monolayer. The flask is incubated to detach cells and then optically observed for cell detachment. If necessary, the flask is tapped gently for cell detachment. 550 mLVP-SFM medium are added to the flask and the cell suspension is equally distributed to 4 sterile 175 mL centrifuge tubes. The cell suspension is then centrifuged as described in Table 6 below. After centrifugation, the supernatant is removed and discarded while the cell pellet is loosened by tapping and re-suspended in 62.5 mLVP-SFM medium each tube by pipetting up and down. The culture is fully suspended if no cell clumps are visible. Cell suspensions are pooled in a sterile container resulting in a final volume of 250 mL and 0.5 mL are removed and used to perform a cell count determining viable cells and viability as disclosed herein.

TABLE 6
Cell harvest parameters-Stage 2

| | | Operating criteria |
|---|---|---|
| Stage | Parameter | (range) |
| Cell harvest | PBS Cell wash volume (10L-CS) | 500 mL |
| Stage 2 | TrypLE select volume (10L-CS) | 250 mL |
| | Cell detachment incubation | 36.5 ± 1° C. |
| | temperature | |
| | Cell detachment incubation time | 5-10 min until |
| | | detachment |
| | VP-SFM added for | 550 mL |
| | centrifugation | |
| | Centrifugation (g) | 300 × g ± 5% |
| | Centrifugation time | 8 minutes |
| | Centrifugation temperature | Room |
| | | temperature |
| | Pellet resuspension media | 250 mL |
| | (VP-SFM) volume | |

General process parameters are not modified during inoculation procedure. The required amount of cells for inoculation of the bioreactor is filled in a transport vessel equipped with tubings for welding. The vessel is aseptically welded to the bioreactor and the cell suspension is transferred into the bioreactor using overpressure. Parameters for inoculation are shown in the Table 7.

TABLE 7
Inoculation parameters-Stage 3

| Stage | Parameter | Operating criteria (range) |
|---|---|---|
| Inoculation of | Available surface area per | 39600 cm2 |
| bioreactor | 9 g Cytodex I | |
| Stage 3 | Seeding density | 1.00 × 10⁴ cells/cm² |
| | Total cell number needed | 3.96 × 10⁸ cells |
| | Seeding culture volume | 100-250 mL |
| | Total volume bioreactor | 3000-3150 mL |

Following inoculation cells were let to attach the microcarrier for 2 hours. Then the bioreactor is sampled in order to microscopically monitor the cellular distribution on the microcarrier and to determine cell counts of cells attached to the microcarrier and in suspension (Counting procedure for cells attached to the microcarrier is shown in section 3.4.4).

The bioreactor was sampled every 24 hours as follows. Using the sample port of the bioreactor 10 mL of microcarrier suspension is withdrawn and discarded. Using syringe 2 and 3, 10 mL sample each are immediately withdrawn and the microcarrier suspensions are transferred to 50 mL tubes. From the analysis tube 1 mL suspension is transferred to one well of a 12-well plate and the attachment/growth of the cells is microscopically analyzed. 10 μL are taken from the analysis tube and the cells in suspension are counted.

The cells in the cell counting tube are let settle down and the supernatant is discarded using a serological pipet. The same amount of crystal violet/citric acid solution is added, well mixed and incubated for at least 30 min. A sample of 10 μL is taken and the released nuclei is counted using a hemacytometer, or alternatively a nucleocounter. Cells are grown under process parameters indicated above for stage 3 and sampled every 24 hours.

Since the medium is depleted during cell growth phase and toxic by-products accumulated, a medium exchange is preferably performed. The stirring, as well as the DO control are stopped, the microcarrier are let to settle down for at least 15 minutes and the spent medium is removed using overpressure. The bioreactor is re-filled with fresh medium and the stirring and the DO control are started again and. Now, the host cells are ready to be infected.

### Example 4: Virus Production

Process parameters for infection phase are shown in Table 8.

TABLE 8
Process parameters-Stage 4

| Stage | Parameter | Operating criteria (range) |
|---|---|---|
| Virus | Total culture volume | 3000 mL |
| Production | Revolutions per minute | 40 |
| Stage 4 | | |
| | Culture media | VP-SFM |
| | Incubation Temperature | 32.5 ± 1° C. |
| | Dissolved oxygen control | >40% |
| | by process air via sparger | |
| | pH control | 7.2 ± 0.2 |
| | by CO2 via overlay | |
| | (to decrease pH) | |
| | and by sodium bicarbonate | |
| | (to increase pH) | |
| | Process Air | Air via overlay, 0.8 mL/min |
| | CO₂ | CO2 via overlay |
| | | with pH control |
| | Air (O₂) | Air (O₂) via sparger |
| | | with DO control |

General process parameters are not modified during infection procedure. The required amount of virus for the infection with a MOI of 0.001 is filled in a transport vessel equipped with tubing for welding. The vessel is aseptically welded to the bioreactor and the seed virus is transferred into the bioreactor using overpressure.

This MOI may again vary depending on the host cell and the MVSS chosen, but can be easily determined after standard pre-testings with the respective host cell and the respective virus. MOIs between 0.0001 and 0.1 are preferable. Notably, the time point for harvest will change depending on the chosen MOI, which can be determined by the skilled person. Virus is grown under parameters listed in Table 8 and sampled every 24 hours.

### Example 5: Clarification

In order to clarify the virus containing cell broth, the stirring and DO control are stopped and the microcarrier are let to settle down for at least 15 min. The cell broth above the microcarrier bed is withdrawn using overpressure and filtered through a 3 μM polypropylene filter (cf. FIG. 1), or a filter cascade consisting of a 50 μM pre-filter and a 3 μM main filter.

The clarified material is transferred to the downstream part directly (processing on one day) or stored at room temperature overnight (processing on two days). Do not store the viral suspension longer than overnight.

The clarification method chosen here can vary depending on material to be clarified and any suitable filtration method can be applied. It is important to consider the polymorphic large surface of the MV. Therefore, suitable filter materials have to be chosen, which do not show unspecific binding of the MV based preparation, which would result in a loss of yield or functionality. As detailed above, centrifugation should be avoided due to the limited scalability thereof under GMP conditions and/or the risk of contaminations associated with this procedure.

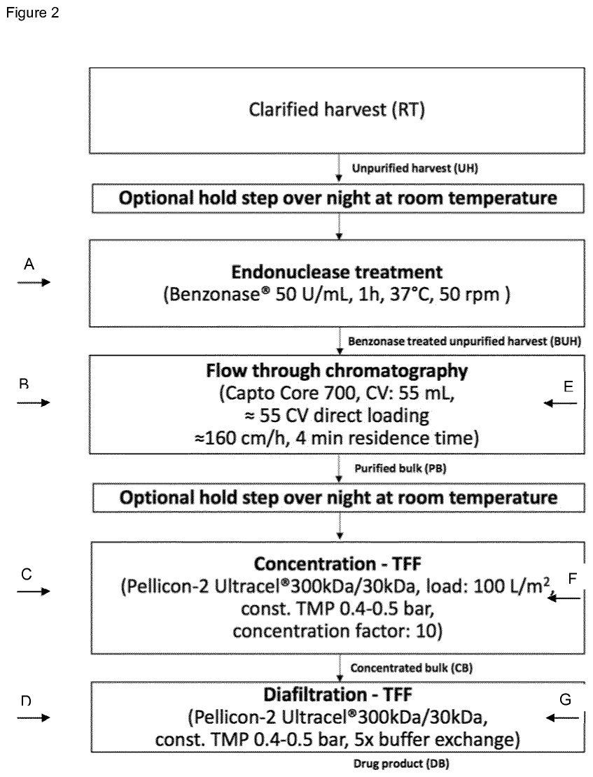

The following examples 6 to 8 relate to downstream manufacturing for MC-CHIK. An overview is provided by way of FIG. 2 showing a corresponding process scheme.

### Example 6: Endonuclease Treatment

Additionally, the protocols disclosed herein can comprise a DNAse treatment step. This treatment can be performed before or after clarifying the virus suspension depending on the host cell and the recombinant infectious virus particle to be purified. A preferred DNAse is a benzonase, but any suitable DNAse having comparable activity, specificity and purity can be chosen for this purpose, whereas the choice of a suitable DNAse can easily be made by a person skilled in the art.

The bag is incubated for 1 h at 37.0±1.0° C. and 50 rpm. The required amount of Benzonase, magnesium chloride and EDTA is calculated to obtain a final concentration of 50 U/mL Benzonase, 2 mM magnesium chloride and 5 mM EDTA in the 4 L bag. Benzonase/1 M magnesium chloride stock solution/500 mM EDTA stock solution is removed from −20.0° C. storage, transported to the cleanroom on dry ice and thawed at ambient/37.0° C. The required amount of magnesium chloride and Benzonase are mixed together into a homogenous solution and then aseptically added to the viral suspension and swirled gently. The final concentration in the viral suspension is 50 U/mL Benzonase and 2 mM magnesium chloride. The bag is incubated at 37.0±1.0° C., 50 rpm for 1-2 hours. The bag is removed from the incubator and the required amount of EDTA is aseptically added to the viral suspension and swirled gently. The final concentration in the viral suspension is 5 mM EDTA.

TABLE 9
Benzonase treatment parameters

| | | Operating criteria |
|---|---|---|
| Stage | Parameter | (range) |
| Material for | Concentration of magnesium chloride | 1M |
| Benzonase | stock solution | |
| treatment | Concentration and pH of EDTA stock | 500 mM, pH 8.0 ± |
| | solution | 0.1 |
| | Benzonase | High purity grade |
| Benzonase | Final concentration of magnesium | 2 mM |
| treatement | chloride in viral suspension | |
| | Final concentration of Benzonase in | 50 U/mL |
| | viral suspension | |
| | Benzonase incubation temperature | 37.0 ± 1.0° C. |
| | Incubation shaker speed | 50 rpm |
| | Benzonase incubation time | 1-2 hours |
| | Final concentration of EDTA in the | 5 mM |
| | viral suspenstion | |

These parameters may vary depending on the DNAse used but can easily be adapted by the person skilled in the art in the knowledge of the present disclosure.

The 4 L bag containing the Benzonase treated virus suspension is immediately transferred to purification. It should be avoided to store the Benzonase treated pool longer than over night.

### Example 7: Flow-Through Chromatography

The chromatography system can be set up as described, exemplarily for a Capto Core 700 26/40 column connected to an ÄKTA Pilot, in the following. The pH electrode is calibrated according to standard procedures. Inlet tubings are connected to the ÄKTA Pilot system inlets A1, A2, B1, B2, B3 and sample inlet S1 are tie wrapped. Using a sterile connective device (SCD), a two T-piece is connected to the inlet tubing on S1 to create a bypass on this line. Using the SCD, cleaning tubing is connected to the inlet tubing on A1, A2, B1, B2, B3 and both lines of the T-piece on S1. Outlet tubing is connected to the ÄKTA Pilot system outlets F1, F3, F5, F7 and each connection is tie wrapped. Air vents are clamped off on the outlet tubing with Kocher clamps. Using the SCD a cleaning outlet tubing set is connected up to outlet tubing on F1, F3, F5 and F7 and to one 10 L waste bag. Using the SCD, the 1 M NaOH solution (>3 L) is connected to the inlet cleaning tubing. All inlets, outlets and the system are flushed with 1 M NaOH. Using the SCD the 1 M NaOH connected to the inlet cleaning tubing is replaced with 0.1 M NaOH (>3 L). All inlets, outlets and the system are flushed with 0.1 M NaOH. Using the SCD the 0.1 M NaOH connected to the inlet cleaning tubing is replaced with WFI (>3 L). All inlets, outlets and the system are flushed with WFI. The Capto Core 700 column is connected aseptically in up flow direction to column position 2. Using the SCD the equilibration buffer (>3 L) is connected to A1 and S1, the regeneration buffer (>3 L) to B1, 1.0 M NaOH (>3 L) to A2, WFI (>3 L) to B2, 20% EtOH (>2 L) to B3. All inlets, outlets and the system are flushed with the respective buffers. When flush is complete, the outlet tubings are emptied by opening the attached air vent. Using the SCD the waste bag on F1 is replaced with a new waste bag. Using the SCD a sterile 4 L bag is connected to F3 to collect the flow-through. Using the SCD a sterile 1 L bag is connected to F5 to collect the wash fraction. Using the SCD a sterile 1 L bag is connected to F7 to collect the regeneration fraction.

TABLE 10
Capto Core 700 column preparation parameters

| Stage | Parameter | Operating criteria (range) |
|---|---|---|
| Connections | Inlets—Buffer | A1, A2, B1, B2, B3 |
| | Inlets—Sample | S1 |
| | Outlets | F1, F3, F5, F7 |
| NaOH flush | Buffer | 1.0M NaOH |
| (1.0M) | Volume | 3 L |
| | Flow rate | 300 mL/min |
| | Flow path | Complete system |
| NaOH flush | Buffer | 0.1M NaOH |
| (0.1M) | Volume | 3 L |
| | Flow rate | Equilibration flowrate |
| | Flow path | Complete system |
| WFI flush | Buffer | WFI |
| | Volume | 3 L |
| | Flow rate | 300 mL/min |
| | Flow path | Complete system |
| Buffer flush | Buffer | Equilibration buffer (A1, S1), |
| | | elution buffer (B1), NaOH |
| | | (A2), WFI (B2), EtOH (B3) |
| | Volume | 3 L |
| | Flow rate | 300 mL/min |
| | Flow path | Respective connections |

Using the above example, the purification is now described. The maximum loading volume is set to 55 CV. A bag comprising the MV particles containing sample is connected to one of the bypass lines of inlet S1, using the sterile connective device (SCD). Inlet S1 is flushed with a minimum of sample material. The bypass on S1 is used to remove air from the line. No air should be introduced into inlet S1 later on. Otherwise only part of the virus material will be processed on the column. Purification is performed as outlined in Table 11 below. Virus flow through is collected in F3 with UV monitoring (start >100 mAU, stop <50 mAU). Maximum delta column pressure is set to 2.0 bar. After completion of the run, all outlets are emptied by opening the air vent on the outlet. The outlet air vents and bags are clamped off and the bags are then disconnected from the outlets using the SCD. Product collection during flow-through is determined by UV280 reading on the UV detector. The collection is started during sample loading when UV280 is >100 mAU and is stopped during the wash step when the UV280 reading is <50 mAU. Product collection parameters are summarized in Table below. The exact volume of the flow-through fraction is noted. Typically the volume of the flow-through fraction (purified bulk material) is 1.0 up to 1.1-fold of the loaded sample volume.

TABLE 11
Virus purification parameters

| Stage | Parameter | Operating criteria (range) |
|---|---|---|
| Final | A1 | Equilibration buffer |
| connection | A2 | 1M NaOH |
| setup | B1 | Regeneration buffer |
| | B2 | WFI |
| | B3 | 20% EtOH |
| | S1 | Sample (Benzonase treated |
| | | unpurified harvest) |
| | F1 | Waste |
| | F3 | Collect flow-through |
| | F5 | Collect wash fraction |
| | F7 | Collect regeneration fraction |
| Equilibration | Buffer | Equilibration buffer |
| of the column | Volume | >500 mL/10 CV |
| | Flow rate/flow velocity | 140 cm/h |
| | Flow path | A1 > Column > F1 |
| | Column flow direction | Normal flow direction |
| Sample loading | Buffer | Sample |
| | Volume | 55 CV |
| | Flow velocity/residence | 140 cm/h/4 min |
| | time | |
| | Flow path | S1 > column > F3 |
| | Column flow direction | Normal flow direction |
| Wash | Buffer | Elution buffer |
| | Volume | 500 mL/10 CV |
| | Flow velocity | 140 cm/h |
| | Flow path | A1 > column > F3 (UV280 > |
| | | 100 mAU)/F5 (UV280 < 50 |
| | | mAU) |
| | Column flow direction | Normal flow direction |
| Regeneration | Buffer | Regeneration buffer |
| | Volume | 500 mL/10 CV |
| | Flow velocity | 140 cm/h |
| | Flow path | B1 > column > F7 |
| | Column flow direction | Normal flow direction |
| Clean column | Buffer | 1M NaOH |
| with NaOH | Volume | 500 mL/10 CV |
| | Flow velocity/contact | 140 cm/h/min. 120 min |
| | time | contact time |
| | Flow path | A2 > column > F1 |
| | Column flow direction | Normal flow direction |
| Clean column | Buffer | WFI |
| with WFI | Volume | 3 L |
| | Flow velocity | 140 cm/h |
| | Flow path | B2 > column > F1 |
| | Column flow direction | Normal flow direction |

The purified bulk material is then transferred to the concentration step (processing on one day) or stored at room temperature overnight (processing on two days).

The buffers used during chromatographic purification correspond to those shown in Table 12. Usually, all buffers are filtered 0.2 μm and de-aerated in a sonicating bath for 15 minutes.

TABLE 12
Buffer preparation

| Stage | Parameter | Operating criteria (range) |
|---|---|---|
| Equilibration | Equilibration | 50 mM HEPES, 150 mM NaCl, pH 7.5 ± 0.1 |
| | buffer | |
| Wash | Equilibration | 50 mM HEPES, 150 mM NaCl, pH 7.5 ± 0.1 |
| | buffer | |
| Regeneration | Regeneration | 50 mM HEPES, 2M NaCl, pH 7.5 ± 0.1 |
| | buffer | |
| Sanitization | | 1M NaOH |

Any other suitable stationary phase material can be used for purification provided that it allows specific retention of the one or more impurities and pass the MV particles through the stationary phase material. An exemplary procedure using an ÄKTA Pilot chromatography system is set up as described above. Any suitable chromatography system can be chosen for the purpose of the present invention provided that it is compatible with the column chosen.

The main peak fraction can additionally be subjected to a further round of purification, polishing or buffer-exchange to separate recombinant infectious virus particles containing genetic material from optionally present virus-like particles.

### Example 8: Virus Concentration and Diafiltration

In this exemplary embodiment, the virus concentration/diafiltration is performed with a sterile Hydrosart, or a Pellicon 2 cartridge (Ultracel®, PLCMK=300 kDa/30 kDa) in a TFF system. During the concentration the purified bulk material obtained from flow-through chromatography purification is fed and the retentate is recirculated to the feed container. The required membrane area is calculated to obtain a maximum feed of 110 L/m². After concentration the diafiltration is performed by adding diafiltration buffer to the feed container at the same rate as permeate is removed from the process. The entire concentration/diafiltration is performed at a constant transmembrane pressure (TMP) of 0.4-0.5 bar.

The conditioning buffer and diafiltration buffer shown in Table 13 are used in the concentration and, respectively, diafiltration step. Prior to purification, all buffers are usually filtered 0.2 μm and de-aerated in a sonicating bath for 15 minutes.

TABLE 13
Buffer preparation

| Stage | Parameter | Operating criteria (range) |
|---|---|---|
| Conditioning | Conditioning | 50 mM HEPES, 150 mM NaCl, pH 7.5 ± 0.1 |
| | buffer | |
| Diafiltration | Diafiltration | 5% Sorbitol (w/v) or PBS for Hydrosart |
| | buffer | |
| Sanitization | Sanitization | 0.1M NaOH |
| | buffer | |

According to a specific embodiment, membrane parameters are as follows: Typical TFF flow rates for Pellicon 2 membranes (C-screen): 540-876 LMH (9.5-14.6 L/min/m²). The flux unit of L/hr/m² is usually abbreviated as LMH, which will also be used herein. Chemical stability Ultracel material: 0.1 M NaOH—contact time 30-60 min, pH compatibility: 2-13. Hold up volume 0.1 m² membrane: upstream: 18 mL; downstream: 10 mL

The TFF system can be set-up as described below. Flush volumes are calculated based on a system hold up volume of approx. 200 mL. Either, a sterile single-use flow path is provided, or the system has to be sterilized using 1 M NaOH. Inlet and outlet tubings (feed, permeate, retentate) are connected using a SCD. Waste bags (20 L) are connected to all outlets. All inlets, outlets and the system are flushed with 1 M NaOH (>5 L). Using the SCD the 1 M NaOH connected to the inlet cleaning tubing is replaced with WFI (>5 L). All inlets, outlets and the system are flushed with WFI (>5 L). The transfer lines are flushed with conditioning buffer and diafiltration buffer (>2 L each). One Pellicon 2 filter capsule is aseptically connected to the TFF system. The membrane is flushed with 100 L/m²WFI with a crossflow rate of 360-600 LMH. A feed tank containing the appropriate amount of WFI is aseptically connected and the feed flow is started. Additional WFI is added to the feed tank if required. One third of the total required WFI amount is flushed through the retentate outlet. Two third of the total required WFI amount is flushed through the permeate outlet. An integrity testing can be optionally carried out using routine procedures. The membrane is pre-conditioned with 50 L/m² conditioning buffer at a crossflow rate of 360-600 LMH. The feed tank is filed with the appropriate amount of conditioning buffer and the feed flow is started at the crossflow rate of 360-600 LMH. One third of the total required volume is flushed through the retentate outlet. Two third of the total required WFI amount is flushed through the permeate outlet. When pre-conditioning is complete, the outlet tubing is emptied by opening the attached air vent. Using the SCD all waste bags are replaced with new sterile bags. The purified bulk material is transferred from the chromatography part to the concentration/diafiltration part.

The concentration is carried out as follows. The feed tank is filled with the purified bulk material and gently stirred at 50 rpm. The feed pump is set at crossflow rate of 240-360 LMH (e.g., 4-6 L/min/m²) for Pellicon 2 cartridges, or 360 to 600 LMH for Hydrosart in full recycle mode. The feed flow, retentate pressure and the permeate flow are adjusted to obtain a constant TMP of 0.4-0.5 bar. The permeate outlet is opened. The TFF is performed until the feed volume is concentrated 10 times. The feed pump is stopped and the permeate port is closed. The permeate waste bag is replaced with a new sterile bag. The concentrated bulk material remains in the feed/retentate recycle tank for the diafiltration. The volume of the concentrated bulk material is 1/10 of the purified bulk material.

After concentration the diafiltration is performed by adding diafiltration buffer to the feed container at the same rate as permeate is removed from the process. The TFF system is configured to the constant volume diafiltration mode. The feed pump is set at crossflow rate of 240-360 LMH, or 360 to 600 LMH for Hydrosart, in full recycle mode. Adjust the feed flow, retentate pressure and the permeate flow to obtain a constant TMP of 0.4-0.5 bar. The permeate outlet is opened and the permeate flow rate is measured. The diafiltration buffer is added to the feed tank at the same flow rate as the permeate is removed. In total 5 diavolumes are used. The end volume is 1/10 of the purified bulk material. The diafiltration is stopped. The MV particles are now present in a formulation buffer and can be transferred to fill.

### Example 9: Generation of Cellular Inoculum/Expression of Measles Viruses (MVs)

An important parameter in process design for anchorage dependent cells is the availability of a robust and scalable seed train. Using classical T-flasks puts a high work load to the operators of such a process and the usage of stirred formats such as spinner flasks and seed bioreactors in combination with microcarrier bear severe problems caused by bead-to-bead transfers. To address these disadvantages, a Vero cell seed train was developed that is fully based on static cultures using multi-layer flasks in different formats. This setup gives a high degree of flexibility and reduces the number of handling steps thereby eliminating the risk of possible process failure. Within 10 days cells for the inoculation of a 3 L bioreactor can be generated starting from a cell bank, using only two steps. Using a similar setup cell numbers can be expanded within 14 days for the inoculation of a 10 L bioreactor in 3 steps. To reduce the number of handling steps, robotics can be implemented into the process for handling of CellStacks to inoculate industrial scale bioreactors in short time frames.

In detail, expression of MVs was performed in Vero cells grown in VP-SFM medium supplemented with 4 mM L-Glutamine and 0.2% (m/v) Kolliphor P188 under serum-free conditions. For passaging, cells were washed with PBS buffer and detached using TrypLE recombinant trypsin.

A vial of a Vero development cell bank was thawed and cell numbers were increased using 5-layer cell culture Multi-Flasks and 10-layer CellStacks. Bioreactor runs were performed in a BioFlo 320 system (Eppendorf, Germany), using a 5-L vessel (1.3-3.8 L working volume) equipped with standard pH, DO and temperature probes and in addition a capacitance probe for enabling online cell density measurements (Hamilton, Switzerland). Temperature was kept at 36.5° C. during growth phase and reduced to 32.5° C. after infection. The pH was set at 7.2, dissolved oxygen was kept at 40% air saturation and agitation was kept at 40 rpm. Cytodex-1 microcarrier concentration (GE Healthcare, Uppsala, Sweden) was set to 3 g/L and the working volume was kept at 3 L.

After cells reached confluence a medium exchange was performed and cells were infected with MV at a multiplicity of infection of 0.001. The genomic vector comprised a nucleic acid construct containing a nucleic acid encoding CHIKV structural protein being operably linked to a nucleic acid encoding the sequence of full-length, infectious antigenomic (+) RNA strand of MV (cf., for example SEQ ID NO:5).

In order to enhance separation of the virus containing supernatant from cellular debris, the microcarrier were allowed to settle down and the supernatant was clarified using a 3 μM Sartopure PP3 filter cartridge (Sartorius, Germany).

Though the examples were carried out using cells and virus particles characterized by a definite sequence of nucleic acids and/or amino acids, the person skilled in the art will readily appreciate that the general concept disclosed herein is fully applicable to other cells and viruses and in particular to large, pleomorphic viruses. Hence, the person skilled in the art is capable to readily select and/or design further cells and virus particles, respectively, having other sequences than those explicitly described herein, which cells and virus particles, respectively, are suited to be used in the context of the method and system of the present invention.

### Example 10: Production of MV Using an STR Setup

Due to the adherent nature of Vero cells a bioreactor should provide specific surfaces for the cells to grow on such as fibre-cell matrices or different types of microcarrier. Microcarrier have in the past been used for cell culture processes and they have been shown to be suitable for the cultivation of Vero cells even at large scales. However, the exact type and concentration of microcarriers remained to be elucidated. Therefore, different types and concentrations of microcarrier were evaluated. 3 g/I Cytodex I turned out to be ideal for growing Vero cells in supplemented VP-SFM medium under serum-free conditions (data not shown).

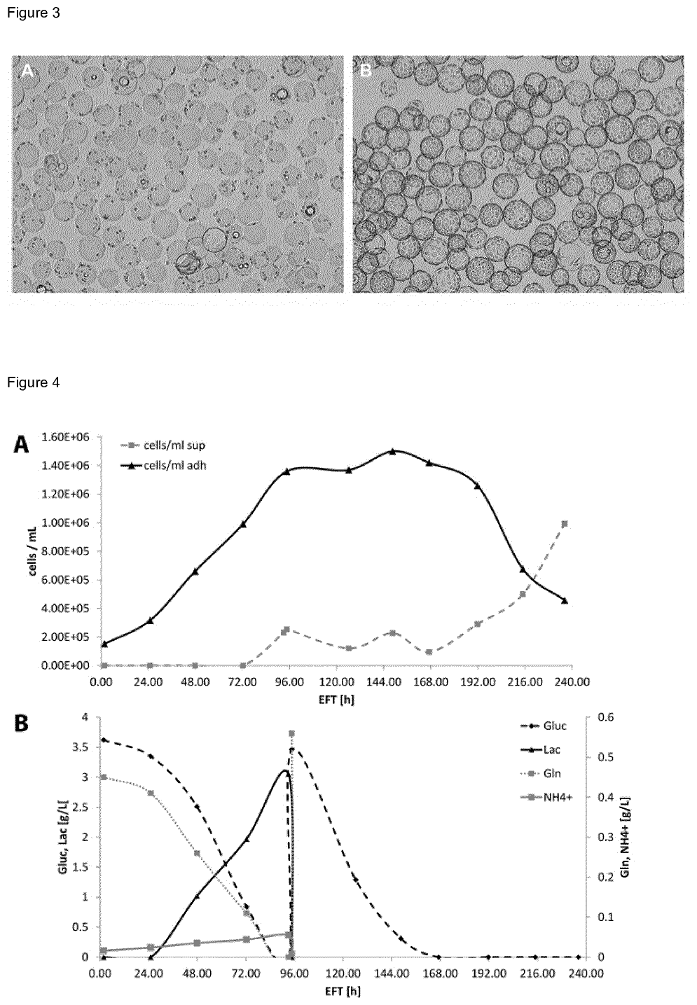

FIG. 3 shows that using the microcarrier in the method of the present invention, cells readily attach to the microcarrier within 1 hour after inoculation and continue to grow until confluence. Different inoculation cells densities were further evaluated, and 1×10⁴ cells/cm² microcarrier surface turned out to be ideal. Using lower numbers would be beneficial regarding seed-train steps but the number of microcarrier not covered with cells would then increase to a level that reduces the overall productivity of the process.

The transfer from static to stirred type culture formats did not affect cell growth. Upon attachment cells immediately started to grow until confluence resulting in a cell density of up to 1.5×10⁶ cells/mL 96 h post inoculation (FIG. 4A). During this growth phase cells are metabolizing nutrients provided by the medium thereby producing toxic by-products such as lactate and ammonium at high levels (FIG. 4B). In order to provide cells with conditions ideal for infection with and production of MV a medium renewal was performed after cell growth declined 96 h post inoculation.

After medium renewal, the temperature of the system was reduced to 32.5° C. in order to prevent a decrease of viral infectivity. Different multiplicity of infections (MOI) were evaluated (data not shown). Infecting the culture with an MOI of 0.001 turned out to be best. Upon infection cells still consumed glucose provided by the fresh medium as shown in FIG. 4B but cellular growth was almost stopped. During the course of infection cells start to detach from the microcarrier and finally lyse. After most cells have lysed the culture was stopped and the virus containing supernatant was clarified using a 3 μM filter. In order to assess productivity of each process run, samples were taken daily and tissue culture infectious dose 50 (TCID50) was determined.

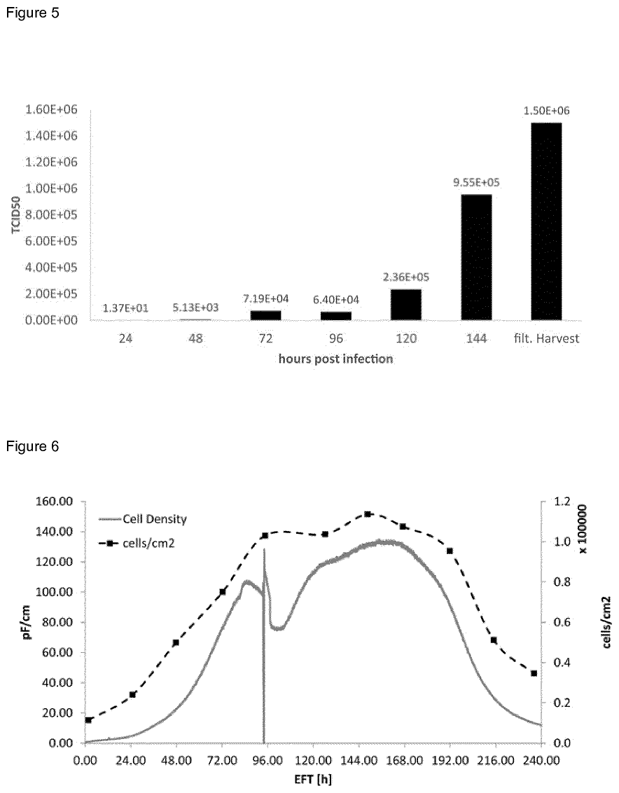

FIG. 5 shows that viral titers increase constantly during production process and reach a top level of up to 1×10⁶ TCID₅₀/mL 144 h post infection. After clarification the titer slightly increased which can be explained by a breakup of viral aggregates during the filtration process.

### Example 11: On-Line Monitoring of STR Based Production Process

When virus particles are produced using host cells attached to a microcarrier, it is difficult to determine cell growth on-line. However, a lack of on-line data limits the possibility to understand and control the process in detail. It has now been surprisingly found that a probe can determine viable cell density based on permittivity measurements, even when the cells are attached to the microcarrier. Viable cells are considered to behave like little capacitors and hence their polarization and depolarization in an alternating electrical field to correlate to the viable cell density. A great advantage of such a principle is that only live cells are measured, so that the measurement is deteriorated neither by cellular debris nor by the microcarrier.

FIG. 6 shows progression of cell densities measured by off-line techniques and on-line permittivity measurements. The on-line signal immediately reacts on the inoculation of the bioreactor and continuously increases upon confluence of the cells on the microcarrier and depletion of the medium. Off-line values are in line with on-line counterparts and deviations can be explained by technical issues during off-line measurements. 96 h post inoculation medium is exchanged and the culture is infected with MV. This causes a stop in cell growth followed by a detachment and lysis of the cells.

Using data provided by the probe, it is possible to precisely determine the ideal infection and harvest time points. A decrease of the signal correlates with a decrease in the total amount of viable cells in the system. Under the prerequisite that all process parameters are within defined specifications, the first decrease of permittivity signals in the course of infection can therefore be defined as ideal point of infection. Similar considerations can be made for harvesting the culture. If the signal falls below a certain threshold most of the cells are dead and the culture is ready for further processing. Predicting the ideal harvest time point is crucial for further downstream processing regarding highest possible yields and lowest possible contaminations due to lysed cells.

### Example 12: Chromatography

Chromatographic experiments were performed on an ÄKTA explorer 100 equipped with a P-960 sample-pump and fraction collector (Frac-950) (GE Healthcare, Uppsala, Sweden). Unicorn software 10.1 was used for control and data acquisition. Conductivity, pH, and absorbance at 280 and 260 nm were monitored simultaneously. Elution fractions were collected by the fraction collector, pooled according to chromatogram and stored at −80° C. For equilibration (mobile phase A) 50 mM HEPES, pH 7.5 was used and for elution and regeneration (mobile phase B) 50 mM HEPES, 2 M NaCl pH7.5. Optional sanitization was performed with 1 M NaOH.

Preliminary experiments were performed with membrane absorbers providing anion-exchange ligands NatriFlo HD-Q Recon (Column volume (CV): 0.8 mL, Natrix Separations, Burlington, ON, Canada) and Sartobind Q (CV: 1 mL, Sartorius Stedium Biotech, Goettingen, Germany). Two resin-based ion-exchangers Toyopearl Sulfate-650F (cation-exchanger, Tosoh Bioscience, Stuttgart, Germany) and Eshmuno Q (anion-exchanger, Merck KGaA, Darmstadt, Germany) and a flow-through chromatography medium, Capto Core 700 (GE Healthcare, Uppsala, Sweden) were tested. Ion-exchange medium was packed in Tricorn 5/100 columns (Eshmuno Q: CV: 1.7-1.9 mL; Toyopearl Sulfate-650F: CV: 2.0 mL) and Capto Core 700 was packed in Tricorn 5/50 columns (CV: 1.1-0.9 mL). All columns were packed according to the medium manufacturer's instructions and loaded with bulk harvest material thawed at 37° C. The detailed experimental performance parameters are summarized in Table 14.

Preparative purifications of MVs were conducted by flow-through chromatography using Capto Core 700 packed in Tricorn 5/50 or XK 16/20 columns (GE Healthcare, Uppsala, Sweden) according to the medium manufacturer's instruction. Bulk harvest material, optionally endonuclease treated, was directly loaded on the column. Equilibration and wash was performed each for 10 CV with 50 mM HEPES, 150 mM NaCl pH 7.5. For regeneration (10 CV) 50 mM HEPES, 2 M NaCl pH 7.5 was used. Optionally, a sanitization step was performed with 1 M NaOH for 10 CV hold. The flow velocity was adjusted to obtain a constant residence time of about 4 min throughout all purifications. The eluted product material was kept at −80° C. for storage.

### Example 13: Preliminary Screening Experiments

Different stationary phases were screened for purification of MV (MVs) allowing a direct loading procedure of filtered (clarified), and optionally endonuclease treated, cell culture supernatant. Two anion-exchange membrane adsorbers (NatriFlo HD-Q Recon and Sartobind Q) and three bead based resins, operated in flow through, cation- or anion-exchange mode (CaptoCore 700, Toyopearl Sulfate-650 F, Eshmuno Q) were tested.

TABLE 14
Summary of preliminary screening experiments

| | Stationary phase | | | | |
|---|---|---|---|---|---|
| | NatriFlo | | Toyopearl | | Capto |
| | HD-Q Recon | Sartobind Q | Sulfate-650F | Eshmuno Q | Core 700 |
| Column | 0.8 | 1 | 2 | 1.7 | 1.1 |
| volume (mL) | | | | | |
| Loading | 50 | 50 | 6.6 | 20 | 20 |
| volume (CV) | | | | | |
| Flow rate | 4 | 10 | 0.5-0.08 | 0.5-0.01 | 0.2 |
| (mL/min) | | | | | |
| Flow velocity | — | — | 153-25 | 155-3 | 62 |
| (cm/h) | | | | | |
| Equilibration | 50 CV 7.5% B | 50 CV 7.5% B | 10 CV 7.5% B | 10 CV 7.5% B | 10 CV 7.5% B |
| Wash | 40 CV 7.5% B | 40 CV 7.5% B | 3.5 CV 7.5% B | 10 CV 7.5% B | 10 CV 7.5% B |
| Elution | SGE: 40 CV each | SGE: 20 CV each | LGE: 7.5-50% B | LGE: 7.5-50% B | — |
| | 7.5/12.5/25/50% B | 7.5/12.5/25/50/75% B | in 20 CV | in 20 CV | |
| Regeneration | 40 CV 100% B | 20 CV 100% B | 10 CV 100% B | 5 CV 100% B | 10 CV 100% B |
| Recovery | <1.0% | <1.0% | 1.2% | 17.0% | 61.1% |
| MV (%) | | | | | |

SGE: Step gradient elution
LGE: Linear gradient elution

Notably, all methods except for the Capto Core 700 procedure were performed in the bind-elute mode as control and to compare performance. NatriFlo has a specific physical structure, as it is a 3-D macroporous hydrogel and thus has a specific structure. Principally, NatriFlo can be use in the bind-elute as well as the flow-through mode.

The purification processes based on both membrane adsorbers and on the cation-exchange medium yielded very low infective virus recoveries 1%; Table 14). When the anion-exchange medium (Eshmuno Q) was used the active MVs were captured from the filtered cell culture supernatant and 17% of infective virus eluted during a linear salt gradient (150-1,000 mM NaCl). Majority of MVs (11.1%) eluted at the end of the gradient at high salt concentrations between (73.4-97.8 mS/cm). Whereas majority of protein impurities did not bind under these conditions and were present in the flow-through and wash fraction (43.5% of total loaded proteins). Majority of dsDNA impurities (63.0% of total loaded dsDNA impurities) eluted at intermediate salt concentrations (17.1-73.4 mS/cm) prior to MVs together with another portion of 32.0% of total loaded proteins. During the wash step and the gradient elution the column back pressure increased and exceeded the maximum column pressure (data not shown). This might primarily be caused by high DNA concentrations. A benzonase or more generally DNAse digestion may thus result in a better viscosity of the material to be purified. Therefore, the flow rate initially used during the loading procedure (0.5 mL/min) could not be kept constant throughout the elution. The flow rate had to be reduced to the minimum of 0.01 mL/min to enable elution of active viruses. In contrast the purification operated in flow-through mode (Capto Core 700) enabled the capture of major contaminants and MVs were collected in the flow-through fraction. This process resulted in a high MVs recovery of 61.1%, in combination with a 75.1% dsDNA and 73.1% total protein impurity depletion. Although, the column back pressure increased exponentially during loading, the maximum column pressure indicated by the manufacturer was not exceeded in this case. Consequently, a consistent flow rate (0.2 mL/min) throughout the purification process was possible.

These screening experiments demonstrated that a chromatography operated in flow-through mode enables high recoveries of infectious virus. In contrast, all tested conventional bind-elute chromatography steps resulted in insufficient recoveries. Based on the above screening experiments results Capto Core 700 was selected as an exemplarily stationary phase material suited for performing chromatography in flow through mode.

### Example 14: Development and Evaluation of the Endonuclease Treatment Step

Majority of free dsDNA impurities can optionally be removed by an endonuclease treatment step, e.g., using Benzonase® endonuclease. For the design and development of a dsDNA digestion procedure the efficiency of two Benzonase® activities (500 U/mL or 50 U/mL) were compared at 37° C. When 500 U/mL were used the DNA level could be reduced by 75.8% from 195 ng/mL to 47 ng/mL. When 50 U/mL were used the DNA level could be reduced by 67.0% from 251 ng/mL to 83 ng/mL. After selection of the appropriate endonuclease concentration different endonuclease incubation temperatures were tested (Table 15).

TABLE 15
Viral titer and dsDNA content during endonuclease treatment
at different incubation temperatures.

| | dsDNA | Viral titer |
|---|---|---|
| Temperatur | (ng/mL) | (log₁₀ TCID50/mL) |
| | before digestion | |
| 37° C. | 2367.9 | 6.5 |
| 4° C. | 188.9 | 5.5 |
| Room temperature | 2455.62 | 6.5 |
| | after digestion | |
| 37° C. | 127.7 | 6.4 |
| 4° C. | 183.9 | 5.5 |
| Room temperature | 224.0 | 6.5 |

Efficiency endonuclease treatment

| | dsDNA reduction | Recovery viral titer |
|---|---|---|
| Temperatur | % | (%) |
| 37° C. | 94.6 | 81.6 |
| 4° C. | 2.7 | >99.9 |
| Room temperature | 90.9 | >99.9 |

Endonuclease treatment is most efficient at 37° C., whereas digestion is extremely slow at 4° C. However, it needs to be considered that digestion at lower temperatures sustains the viral infectivity. Furthermore, different stop reagents were tested and the effect on the virus infectivity and the dsDNA content were compared. Addition of 5 mM EDTA did not affect the virus infectivity and the amount of dsDNA could be reduced by 94% at 37° C. within one hour of incubation time. An endonuclease step may be accomplished at a variety of different temperatures depending on the enzyme used and the material to be treated. A digestion at lower temperatures (room temperature, or even below) can thus also be suitable.

### Example 15: Flow-Through Chromatography

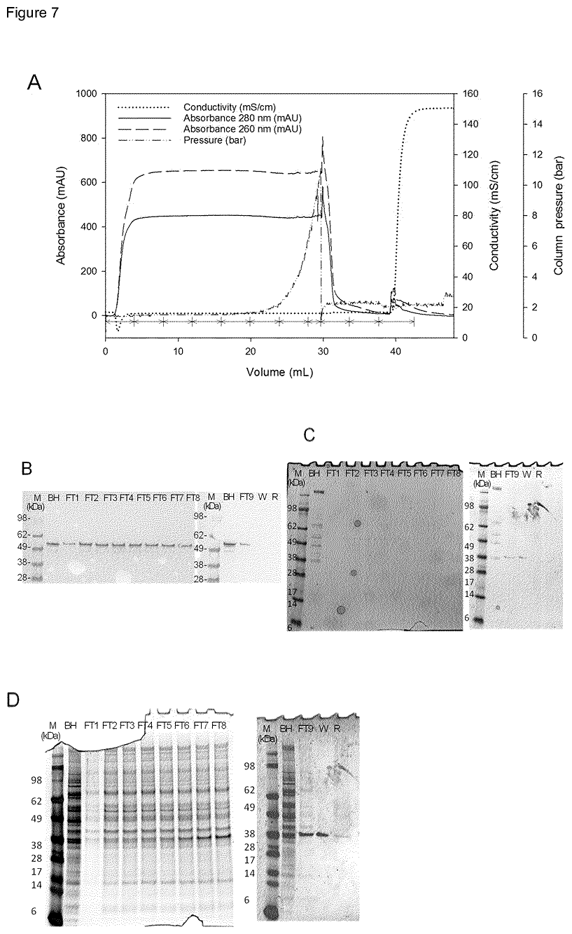

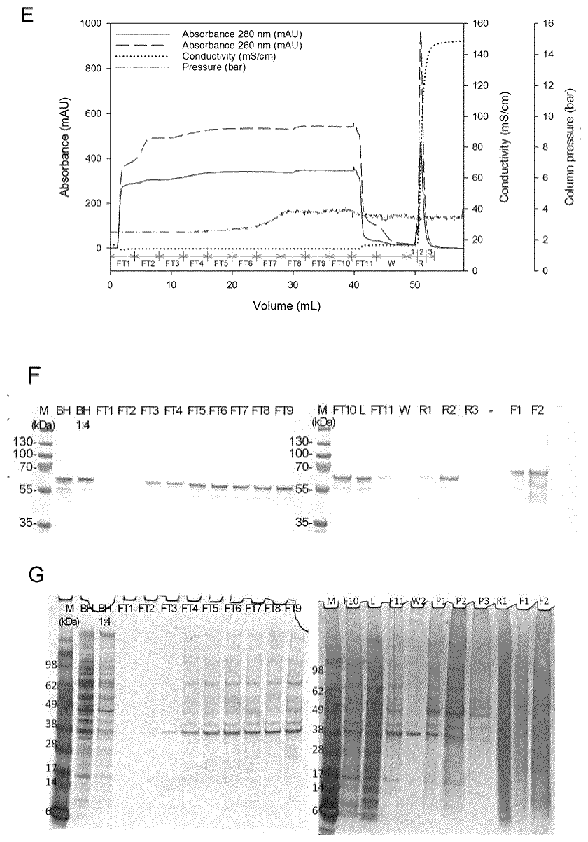

A flow-through chromatography procedure was developed to purify MVs and capture majority of impurities directly from the filtered cell culture supernatant. Preliminary experiments were operated at 1 mL scale, using Tricorn 5/50 columns. The filtered cell culture supernatant was either directly loaded onto the stationary phase material (FIGS. 7A and 7B) or a previous endonuclease treatment step was included (FIGS. 7C and 7D). The MVs passed through the stationary phase material and did not bind thereto. The Western blot analysis (FIGS. 7B and 7D) and the infectivity measured by TCID50 (Table 16) demonstrated that majority of infective MVs eluted in the flow-through fractions, whereas the majority of protein and dsDNA impurities were captured.

TABLE 16
Mass balance of MVs purification by flow-through chromatography in 1 mL scale
using Tricorn 5/50 columns. Loading material was (A) solely filtered or (B) filtered and
endonuclease treated cell culture supernatant.

| | | | | | | | | |
|---|---|---|---|---|---|---|---|---|
| | Viral titer | | Total | | Total protein/10⁵ | | | dsDNA/10⁵ |
| Volume | (log₁₀ | | protein | | inf. particles | dsDNA | | inf. particles |
| (mL) | TCID50/mL) | % | (μg/mL) | % | (μg/1 × 10⁵ part.) | (ng/mL) | % | (ng/×10⁵ part.) |

A) Flow-through chromatography with filtered cell culture supernatant

| | | | | | | | | | |
|---|---|---|---|---|---|---|---|---|---|
| BH = Load | 29.7 | 6.6 | 100.0 | 123 | 100.0 | 3 | 2161 | 100.0 | 59 |
| FT (1-9) | 32.0 | 6.0 | 27.6 | 30 | 26.7 | 3 | 614 | 30.6 | 66 |
| W | 4.0 | 4.4 | 0.1 | 32 | 3.5 | 140 | 23 | 0.1 | 100 |
| R | 6.0 | 4.3 | 0.1 | 36 | 5.9 | 187 | 2440 | 22.8 | 12776 |
| Mass balance | | | 27.8 | | 36.1 | | | 53.6 | |
| chromatography | | | | | | | | | |

B) Flow-through chromatography with filtered and enduculase treated cell culture supernatant

| | | | | | | | | | |
|---|---|---|---|---|---|---|---|---|---|
| BH | 32.2 | 6.8 | 100.0 | 81 | 100.0 | 1 | 2118 | 100.0 | 32 |
| Load | 39.7 | 6.2 | 29.6 | 100 | 152.0 | 6 | 80 | 4.6 | 5 |
| FT (1-11) | 43.7 | 5.8 | 13.9 | 17 | 27.8 | 2 | 40 | 2.5 | 6 |
| W | 5.0 | 3.9 | 0.0 | 12 | 2.2 | 134 | 0 | 0.0 | 5 |
| R | 4.5 | 4.4 | 0.1 | 9 | 1.6 | 34 | 151 | 1.0 | 555 |
| Mass balance | | | 14.0 | | 31.7 | | | 3.5 | |
| chromatography | | | | | | | | | |

The process including an endonuclease treatment step prior to the flow-through chromatography resulted in viruses of even higher purity compared to the purification without an endonuclease treatment step. Furthermore, the column pressure increase during the loading could thereby be minimized.

In both cases the protein level of the virus (FT) fraction could be reduced to a comparable level of 3 μg/1×10⁵ infective particles (without endonuclease treatment) and 2 μg/1×10⁵ infective particles (with endonuclease treatment), whereas the dsDNA level in the virus fraction could be decreased by 11-fold, from 66 ng/1×10⁵ infective particles to 6 ng/1×10⁵ infective particles, only when applying the endonuclease treatment step.

Overall, the recovery of MVs was only between 27.6 and 13.9% and no closed mass balance could be obtained. This indicates that MVs were tightly bound to the chromatography medium or to other column/system components. To investigate this phenomenon, the used column filter frits, were treated with reducing agents for 30 min at 100° C. (F1: LDS-sample buffer; F2: SDS-running buffer) to dissolve strongly bound compounds. Western blot analysis of this fractions (FIG. 7D samples F1 and F2) proved the presence of MVs proteins, suggesting the adsorption of MVs to the filter frit material. In this case quantification of MVs by TCID50 was not possible because of the harsh reducing agents which had to be used for the extraction. To circumvent the usage of filter based frit material the column hardware equipment was substituted from Tricorn (e.g., 7 μm porosity used herein, but also other formats can be used) to XK columns, which provide net based frits instead of filter based ones. Using a net instead of a filter, may have the advantage that the viruses cannot bind in an unspecific manner to the material. Still, suitable filters not showing unspecific binding and having an adequate pore size are also suitable.

The materials used were thus: Tricorn with a 7 μm porosity—material: Ethylene propylene diene (EPDM)*Polyethylene (PE); and XK column: 10 μm net porosity—Polypropylene (PP) (reinforced glass fiber), Polyamide (PA). Still, it has to be noted that porosity might be measured differently by different manufacturers.

TABLE 17
Mass balance of MVs purification by flow-through chromatography in 1
mL scale using a XK 16/20 column. The loading material was filtered
and endonuclease treated cell culture supernatant.

| | | Viral titer | | Total | | Total protein/10⁵ | | | dsDNA/10⁵ |
|---|---|---|---|---|---|---|---|---|---|
| | Volume | (log₁₀ | | protein | | inf. particles | dsDNA | | inf. particles |
| | (mL) | TCID50/mL) | % | (μg/mL) | % | (μg/1 × 10⁵ part.) | (ng/mL) | % | (ng/×10⁵ part.) |
| BH | 27.9 | 5.6 | 100.0 | 214 | 100.0 | 55 | 196 | 100.0 | 50 |
| Load | 34.4 | 5.4 | 85.9 | 173 | 99.8 | 63 | 38 | 24.3 | 14 |
| FT | 38.4 | 5.4 | 95.0 | 23 | 14.5 | 8 | 21 | 14.8 | 8 |
| W | 4.0 | 4.1 | 0.4 | nd | — | — | nd | — | — |
| R | 6.5 | 1.7 | 0.0 | nd | — | — | 5 | 0.6 | 8626 |
| Mass balance | | | 95.4 | | 14.5 | | | 15.4 | |
| chromatography | | | | | | | | | |

n.d.: not detectable

Using the XK column housing enabled the elution of the entire loaded amount of MVs and a closed mass balance could be calculated (Table 17). However, 85.3% of total loaded protein and the 8.9% of total loaded dsDNA impurities could not be regenerated from the column using 2 M NaCl or even 1 M NaOH. This indicates strong hydrophobic or irreversible binding of those impurities to the stationary phase which cannot be altered by aqueous buffer conditions. Only a regeneration procedure containing 1 M NaOH and 30% Isopropanol enabled the full regeneration of residual impurities (data not shown).

### Example 16: Scale Up

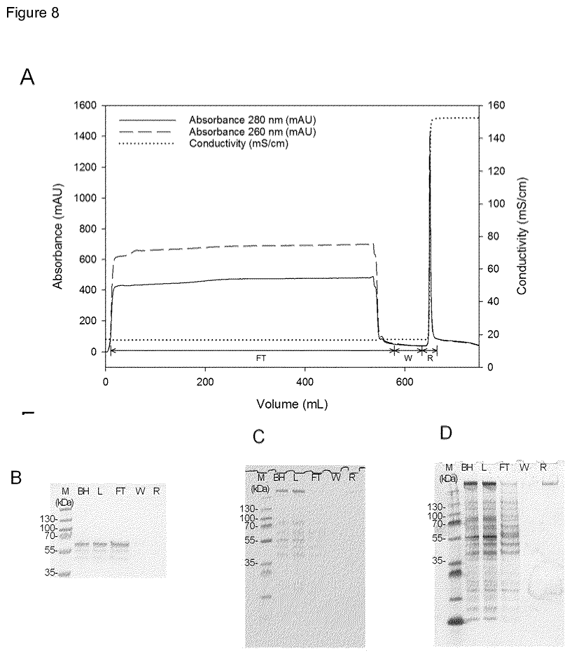

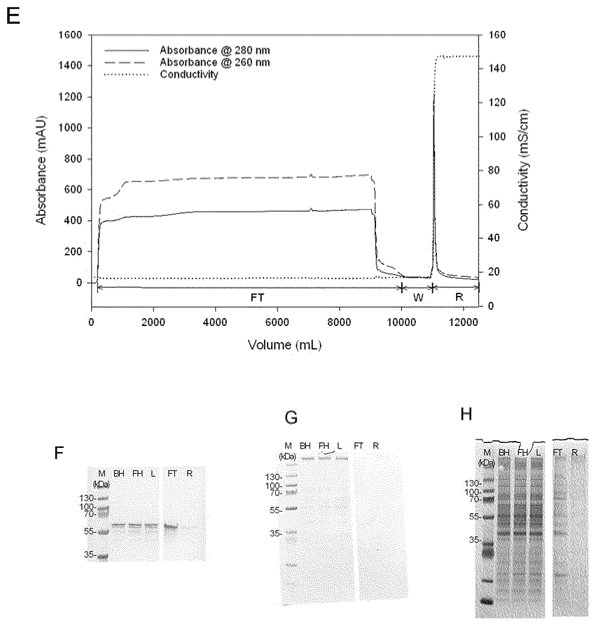

Based on the results gained during process development the process was scaled up to a 10.5 mL and 188.5 mL column keeping the residence time of 4 min constant. Reproducible process performance was demonstrated for four individual runs for purification of three different starting material batches (A, B and C). A typical example chromatogram at 10 mL scale and 188 mL scale and the corresponding Western blot analysis is shown in FIG. 8. A detailed summary of all three runs is presented in Table 18.

TABLE 18
Summary of MVs purification by flow-through chromatography in three
individual runs using starting material batch A, B and C.

| | | Purification run | | | |
|---|---|---|---|---|---|
| | | 1 | 2 | 3 | 4 |
| | | BH batch | | | |
| | | A | B | B | C |
| Chromatographic | Column | XK 16/20 | XK 16/20 | XK 16/20 | XK 50/30 |
| parameters & | Column volume | 10.25 | 9.25 | 10.46 | 188.50 |
| column characteristic | (mL) | | | | |
| | Loading volume | 48 | 56 | 51 | 48 |
| | (CV) | | | | |
| | Flow velocity | 75 | 69 | 78 | 144 |
| | (cm/h) | | | | |
| | Residence time | 4.1 | 4 | 4 | 4 |
| | (min) | | | | |
| Product | BH titer (log₁₀ | 5.4 | 5.3 | 5.1 | 5.1 |
| | TCID50/mL) | | | | |
| | Load titer (log₁₀ | 5.4 | 5.1 | 5.2 | 5.5 |
| | TCID50/mL) | | | | |
| | Final titer (log₁₀ | 5.3 | 4.8 | 5.2 | 5.4 |
| | TCID50/mL) | | | | |
| | Recovery (%) | 73.7 | 36.6 | >99.9 | >99.9 |
| Purity | BH total protein | 93 | 158 | 289 | 185 |
| | (μg/1 × 10⁵ part.) | | | | |
| | Load total protein | 86 | 224 | 174 | 75 |
| | (μg/1 × 10⁵ part.) | | | | |
| | Final total protein | 20 | 66 | 37 | 7 |
| | (μg/1 × 10⁵ part.) | | | | |
| | Overall total | 84.0 | 84.8 | 82.5 | 92.3 |
| | protein depletion | | | | |
| | (%) | | | | |
| | BH dsDNA | 96 | 71 | 149 | 261 |
| | (ng/1 × 10⁵ part.) | | | | |
| | Load dsDNA | 29 | 55 | 40 | 72 |
| | (ng/1 × 10⁵ part.) | | | | |
| | Final dsDNA | 26 | 55 | 29 | 50 |
| | (ng/1 × 10⁵ part.) | | | | |
| | Overall dsDNA | 80.1 | 72.0 | 73.2 | 57.9 |
| | depletion (%) | | | | |

In summary, the process enables consistent and reproducible total protein depletion between 82.5 and 92.3% and dsDNA depletion between 57.9 and 80.1%. The residual dsDNA content in the final MVs fraction varies from 26 to 55 ng dsDNA/1×10⁵ infective particles (Table 18). The total protein content in the MVs varies from 7 to 66 μg total protein/1×10⁵ infective particles (Table 18). The MVs concentration stays constant during the chromatographic step and typically the total amount of loaded virus was fully recovered. Deviations of the impurity level normalized to 1×10⁵ infective particles might be ascribed to the variations in the virus titer determination. In general, the methodological error of TCID50 values which is within 0.5 log₁₀ TCID50/mL.

### Example 17: Ultrafiltration/Diafiltration

After chromatographic purification and depletion of majority of impurities the process volume was reduced and the buffer composition was adjusted during an ultrafiltration/diafiltration step by tangential flow filtration. Three ultrafiltration membranes, one hollow fiber (HF) module, one membrane cassette based on composite regenerated cellulose (RC) and one membrane cassette based on stabilized cellulose based membrane (SC) were compared in terms of productivity and final product purity.

More specifically, ultrafiltration/diafiltration was operated by ÄKTA flux s (GE Healthcare, Uppsala, Sweden). 300 kDa composite regenerated cellulose (RC) cassettes (Pellicon® XL, Merck KGaA, Darmstadt, Germany), 300 kDa polysulfone hollow fibre (HF) modules (Start AXM Cartridge, GE Healthcare, Uppsala, Sweden) and 30 kDa stabilized cellulose (SC) based membranes (Hydrosart®, Sartorius, Gottingen, Germany) were tested. Additionally, 100 kDa stabilized cellulose (SC) based membranes (Hydrosart®, Sartorius, Gottingen, Germany) were tested for certain MV preparations which yielded better concentrations rates (around 50- to 100-fold) in direct comparison to the 30 kDa SC based membrane for certain MV preparations and a higher purity. A membrane area of 50 cm² was tested and the membrane feed was kept constant at 110 L/m². The transmembrane pressure was kept constant throughout the process between 0.4 to 0.5 bar by adjusting a retentate pressure control valve. Membranes were pre-treated and flushed as described in the particular manufacturer's instructions before the experiments. Feed material was chromatographically pre-purified using Capto Core 700 and frozen material was thawed at 37° C. Throughout the filtration experiments samples were collected from the feed material (F), the concentrate (C) and the permeate (P) at stored at −80° C.

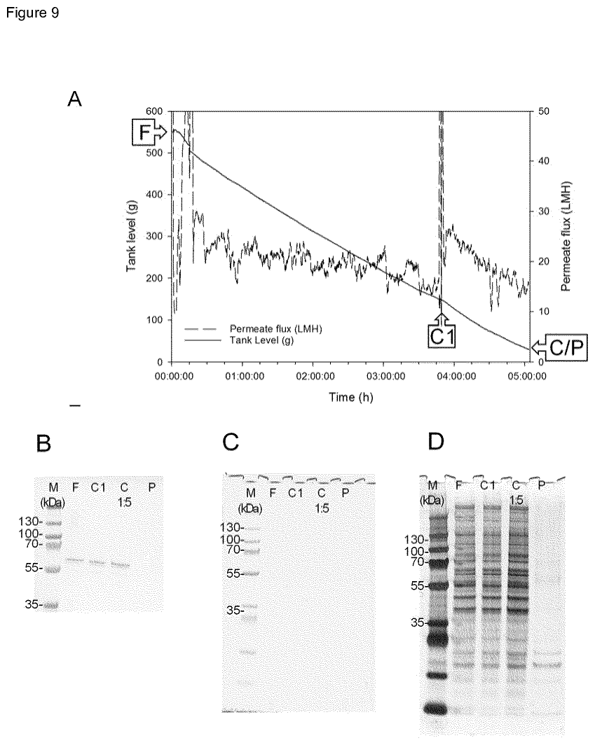

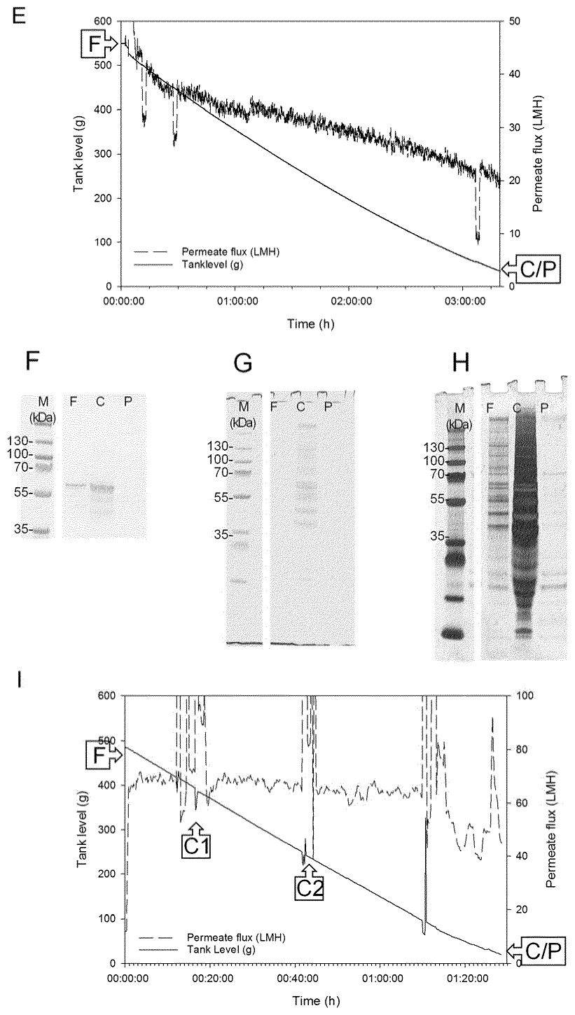

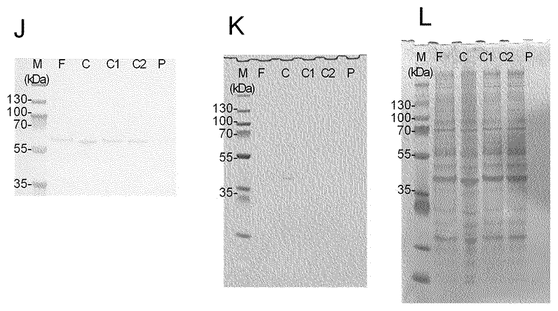

The ultrafiltration and diafiltration was performed at a constant, low transmembrane pressure (0.4-0.5 bar) to maintain conditions which do not harm the shear-sensitive viruses. Consequently, the permeate flux was decreased during the filtration. The volume of the virus material purified by flow-through chromatography was reduced about 12-fold using the HF module (FIGS. 9A and 9B) and about 9-fold using the RC membrane (FIGS. 9C and 9D).

TABLE 19
Comparison of ultrafiltration MVs material by HF module, RC
membrane and SC membrane.

| | | Membrane type | | |
|---|---|---|---|---|
| | | HF | RC | SC |
| | | Membrane area (cm²) | | |
| | | 50 | 50 | 50 |
| Volume | Total dead volume (membrane & | 15 | 33 | 32 |
| | system) (mL) | | | |
| | Feed volume (mL)* | 565 | 583 | 496 |
| | Concentrate volume (mL)* | 45 | 63 | 53 |
| | Volume reduction factor* | 12.6 | 9.3 | 9.4 |
| | Throughput (L/m²/h)* | 21.7 | 34.3 | 71.4 |
| Product | Titer Feed (log₁₀ TCID50/mL) | 5.2 | 5.3 | 5.9 |
| | Titer Concentrate (log₁₀ TCID50/mL) | 5.9 | 6.2 | 6.9 |
| | Concentration factor | 5.4 | 8.5 | 12.5 |
| | Step recovery (%) | 43.9 | 99.9 | >99.9 |
| Purity | Feed total protein (μg/1× 10⁵ part.) | 33 | 23 | 25 |
| | Concentrate total protein (μg/1 × 10⁵ | 38 | 27 | 13 |
| | part.) | | | |
| | Massbalance total protein (%)† | 58.3 | >99.9 | 75.9 |
| | Feed dsDNA (ng/1 × 10⁵ part.) | 33 | 36 | 108 |
| | Concentrate dsDNA (ng/1 × 10⁵ part.) | 64 | 40 | 56 |
| | Massbalance dsDNA (%)† | 92.2 | >99.9 | 69.7 |

*Total dead volume was included for calculation
†Massbalance = concentrate + permeate

As shown in Table 19, when the cellulose based membranes (RC and SC) were used the entire loaded virus amount could be recovered again. The viral titer was concentrated 8.5 up to 12.5 times. The highest average throughput of 71.4 LMH was achieved using the SC membrane, resulting in significantly reduced process time in comparison to HF module and RC membrane. During diafiltration for five volume exchanges the viral titer was not significantly affected in any of these systems. It has to be noted that RC/SC and HF structurally represent completely different materials. HF is structured as fibres, whereas the cellulose-based RC/SC are flat sheets which may be packed in cassettes,

### Example 18: Process Summary

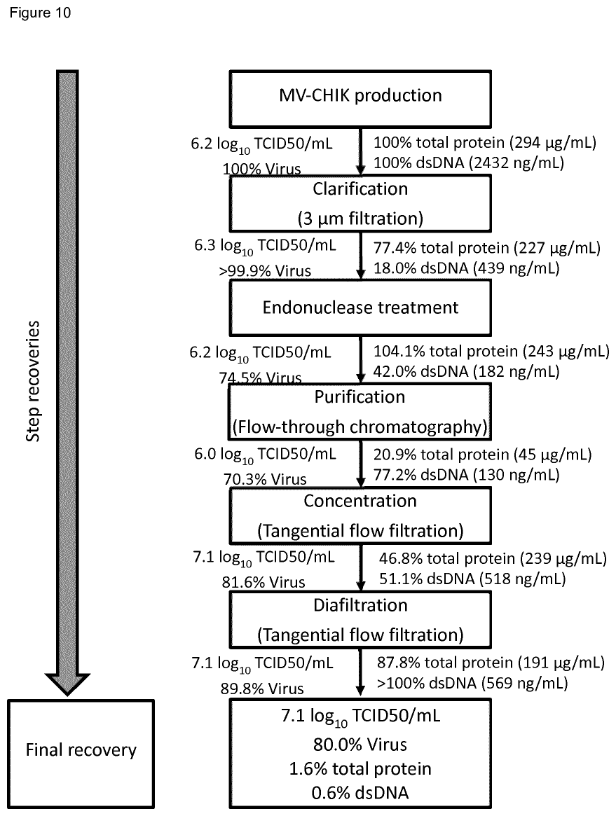

A summary of the above exemplified process is shown in FIG. 10, which shows a process scheme according to an embodiment of the method of the present invention. Recoveries (yields) in terms of TCID50/mL as well as relative recoveries calculated from TCID50/mL are indicated for each step. Furthermore, relative amounts of impurities, specifically total protein content and dsDNA content, are indicated. As it is known in the relevant technical field, the calculation of TCID50 values can be associated with a certain error range (0.5 log). For the determination of impurities (protein, DNA etc.) rather sensitive determinations methods are available.

### Example 19: Testing

In examples 1 to 18, reference is made to the analysis techniques, which were carried out as follows unless otherwise dictated by context.

Sodium Dodecyl Sulfate-Polyacrylamide Gel Electrophoresis (SDS-PAGE) and Western Blot Analysis

For electrophoresis under reducing conditions NuPAGE Bis/Tris gels 4-12% (Invitrogen, Carlsbad, Calif., USA) and MES-SDS running conditions were used in accordance to manufacturer's instructions. If required, samples were diluted in deionized water to obtain similar protein concentrations. SeeBlue® Plus2 Pre-stained Protein Standard (Invitrogen, Carlsbad, Calif., USA) or Page Ruler Plus Prestained Protein Ladder (Thermo Fisher Scientific, Waltham, Mass. USA) was used as the protein molecular weight ladder. Protein bands were stained by Coomassie Brilliant Blue G-250 based EZBIue™ Gel Staining Reagent (Sigma Aldrich, St. Louis, Mo., USA) or by silver using a silver staining procedure as described by Heukeshoven et al. [3]. After SDS-PAGE, proteins were transferred onto a 0.2 μm nitrocellulose membrane using a Trans-Blot® Turbo™ Transfer System with Trans-Blot Turbo Mini Nitrocellulose Transfer Packs (Bio-Rad Laboratories, Hercules, Calif., USA). After electro blotting the membrane was blocked with 3% BSA in PBS-T (0.1% w/v Tween-20 in PBS) for 2 h at room temperature. For detection a mouse monoclonal antibody against Measles NP (3E1) (Santa Cruz Biotechnology, Dallas, Tex., USA) with a dilution of 1:1000 (in PBS-T containing 1% BSA) was incubated for 2 h at room temperature. The secondary antibody, anti-mouse IgG conjugated with alkaline phosphatase (Sigma Aldrich, St. Louis, Mo., USA), was diluted 1:1000 in PBS-T with 1% BSA and was incubated for 1 h at room temperature. Visualization of bands carried out by BCIP®/NBT solution (Sigma Aldrich, St. Louis, Mo., USA), according to manufacturer's instructions. Wash steps between individual steps were performed three times with 1% BSA in PBS-T for 10 min each.

Total Protein Concentration and dsDNA Concentration

The total protein concentration was determined by Bradford assay using Coomassie blue G-250-based protein dye reagent (Bio-Rad Laboratories, Hercules, Calif., USA). The calibration curve was obtained by bovine serum albumin (BSA) standards diluted in TE-Buffer. DNA content was determined by Quant-iT™ PicoGreen® dsDNA kit (Life technologies, Waltham, Mass., USA). Protein and DNA assays were performed according to the particular manufacturer's instructions in 96-well plate format. Signals were measured by Infinite F220/M200 PRO plate reader (Tecan, Männedorf, Switzerland).

Tissue Culture Invective Dose 50 (TCID50)

For determination of a virus sample two 96 well plates were seeded with 5000 Vero cells/well and incubated at 37° C. for 1-2 hours. In the meantime, virus dilutions were performed. Therefore, a 96 well plate containing 200 μL VP-SFM medium supplemented with antibiotics each well was prepared. 50 μL virus sample was applied to row 1, mixed thoroughly and serially diluted to row 11. Row 12 served as a negative control. The cells were infected by addition of 20 μL virus dilution/well and incubated at 37° C. over night. 100 μL supplemented VP-SFM medium was added to each well and then incubated for another 6 days. Each well was observed under a microscope for cytotoxicity or syncytia formation, if one was present the well was noted positive. With the numbers of positive and negative wells per plate a TCID 50 titer was calculated using the Spearman-Kärber formula.

Host Cell Protein (HCP) Concentration

The HCP concentration was measured by a Vero Cell HCP ELISA (Cygnus Technologies, Southport, N.C., USA) according to the manufacturer's instructions. Signals were measured by Infinite F220/M200 PRO plate reader (Tecan, Männedorf, Switzerland).

Endonuclease Treatment

The sample was treated for 1 h with Benzonase purity grade II (Merck KgA, Darmstadt Germany) at a final concentration of 50 U/mL or 500 U/mL in the presence of 2 mM MgCl₂ at 37° C. and 50 rpm on a shaker. Digestion was stopped during preliminary experiments by (A) 5 mM EDTA, (B) 25 mM HEPES, 1.8 M (NH₄)₂SO₄, pH 7.5, (C) 5 mM HEPES, 150 mM NaCl, pH 7.5 or (D) H₂O. The standard endonuclease treatment procedure used for preparative purposes (50 U/mL for 1 h at 37° C. and 50 rpm) was topped by 5 mM EDTA.

Transmission Electron Microscopy (TEM)

Copper grids (400-mesh, Agar Scientific Ltd, Stansted, UK) coated with Pioloform film and shaded with carbon were incubated with samples for 1 min. Sample fixation was carried out with 2.5% glutaraldehyde solution for 15 min and was followed by three wash steps with water. Samples were stained with 1% uranyl acetate in 100 mM cacodylate buffer, pH 7.0 for 30 seconds [4]. The negatively stained and air dried specimens were analysed in a Tecnai G2 200 kV transmission electron microscope (FEI, Eindhoven, The Netherlands), operating at 80 keV.

### Example 20: Viscosity of MV-CHIK Containing Loads

Cell culture media and buffers were purchased from Thermo Fisher Scientific (Waltham, USA). Plastic ware used in cell culture was purchased from Corning (New York, USA). All chemicals were purchased from Merck KGaA (Darmstadt, Germany) or Sigma Aldrich (St. Louis, Mo., USA).

Expression of MVs was performed in Vero cells grown in VP-SFM medium supplemented with 4 mM L-Glutamine and 0.2% (m/v) Kolliphor P188 under serum-free conditions. For passaging, cells were washed with PBS buffer and detached using TrypLE recombinant trypsin.

A vial of a Vero development cell bank was thawed and cell numbers were increased using 5-layer cell culture Multi-Flasks and 10-layer CellStacks. Bioreactor runs were performed in a BioFlo 320 system (Eppendorf, Germany), using a 5-L vessel (1.3-3.8 L working volume) equipped with standard pH, DO and temperature probes and in addition a capacitance probe for enabling online cell density measurements (Hamilton, Switzerland). Temperature was kept at 36.5° C. during growth phase and reduced to 32.5° C. after infection. pH was set at 7.2, dissolved oxygen was kept at 40% air saturation and agitation was kept at 40 rpm. Cytodex-1 microcarrier concentration (GE Healthcare, Uppsala, Sweden) was set to 3 g/L and the working volume was kept at 3 L.

After cells reached confluence a medium exchange was performed and cells were infected with MV at a multiplicity of infection of 0.001. In order to separate the virus containing supernatant from cellular debris, the microcarrier were allowed to settle down and the supernatant was clarified using a 3 μM Sartopure PP3 filter cartridge (Sartorius, Germany).

The sample was treated for 1 h with Benzonase purity grade II (Merck KgA, Darmstadt Germany) at a final concentration of 50 U/mL in the presence of 2 mM MgCl₂ at room temperature and 50 rpm. Digestion was stopped by adding 5 mM EDTA.

Chromatographic experiments were performed on an ÄKTA explorer 100 equipped with a P-960 sample-pump and fraction collector (Frac-950) (GE Healthcare, Uppsala, Sweden). Unicorn software 10.1 was used for control and data acquisition. Conductivity, pH, and absorbance at 280 and 260 nm were monitored simultaneously. Preparative purifications of MVs was conducted by flow-through chromatography using Capto Core 700 packed in XK 16/20 columns (GE Healthcare, Uppsala, Sweden) according to the medium manufacturer's instruction. Bulk harvest material, was filtered and endonuclease treated and directly loaded on the column. Equilibration and wash was performed for 10 CV with 50 mM HEPES, 150 mM NaCl pH 7.5. The flow velocity during purification was adjusted to obtain a constant residence time of about 4 min throughout all purifications. The purified material was collected during the flow-through fraction and was stored at −80° C.

Ultrafiltration/diafiltration was operated by ÄKTA flux s (GE Healthcare, Uppsala, Sweden). Using a 30 kDa stabilized cellulose (SC) based membrane (Hydrosart®, Sartorius, Gottingen, Germany) at a membrane feed of 110 L/m². The transmembrane pressure was kept constant throughout the process between 0.4 to 0.5 bar by adjusting the retentate pressure control valve. Membrane were pretreated and flushed as described in the manufacturer's instructions before the experiments. Feed material was chromatographically pre-purified using Capto Core 700 and frozen material (−80° C.) was thawed at 37° C. After 10-fold concentration the concentrated material was diafiltrated for 5 volume-exchanges to PBS.

Viscosity of process fluid was measured at room temperature by DV-II+Pro viscometer (Brookfield Engineering Laboratories, Middleboro, Mass., USA). Shear stress and corresponding shear rate data were used for evaluation of viscosity by power-law according to Ostwald-de Waele. The viscosities measured at a shear rate of 525 1/sec were selected for comparison.

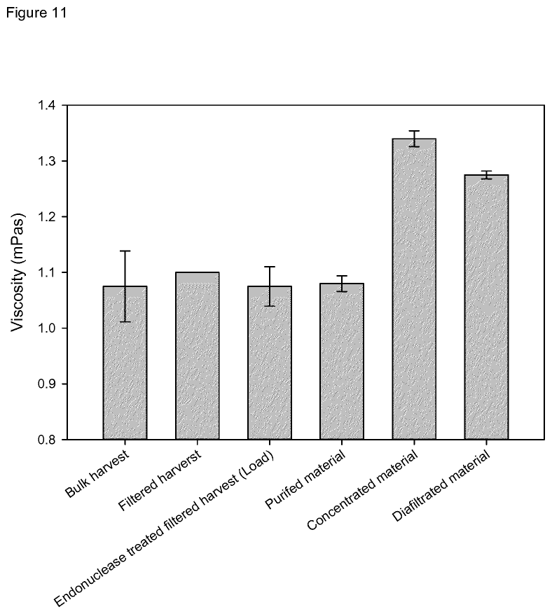

As explained in the general description, the viscosity of the process stream is a critical process parameter during downstream processing, influencing the process control and process sequence. The viscosity of the individual process streams after each unit operation was analyzed and results are summarized in FIG. 11.

The results demonstrated that the viscosity of concentrated material (concentrated and diafiltrated material) is significantly higher compared to the non-concentrated process fluid (bulk harvest, filtered harvest, endonuclease harvest, purified material).

### Example 21: Purification of MV-GFP

Next, purification of MV-GFP is described using embodiments of the method of the present invention. GFP co-expressed—here as soluble protein and not as virus like particle—with MV (scaffold) proteins can serve as excellent fluorescent marker in evaluating success of infection. Afterwards, it is an impurity that needs to be removed by the downstream process.

Cell culture media and buffers were purchased from Thermo Fisher Scientific (Waltham, USA). Plastic ware used in cell culture was purchased from Corning (New York, USA). All chemicals were purchased from Merck KGaA (Darmstadt, Germany) or Sigma Aldrich (St. Louis, Mo., USA).

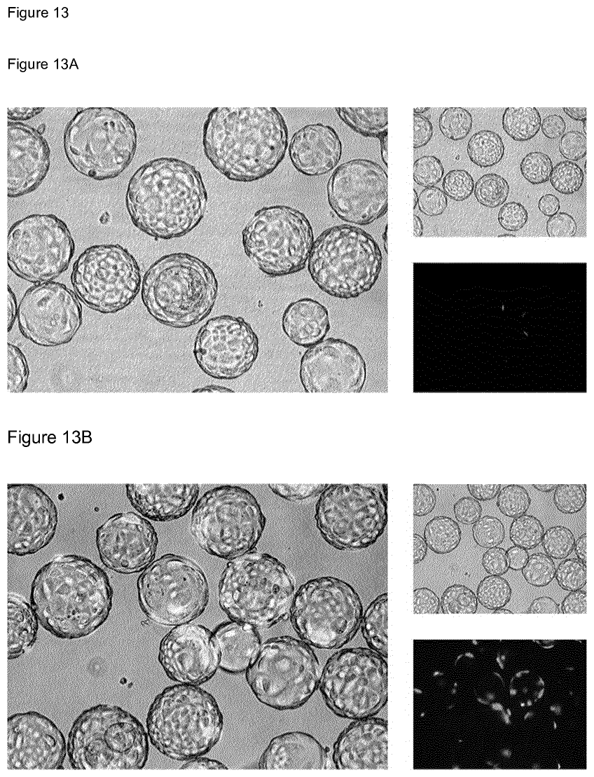

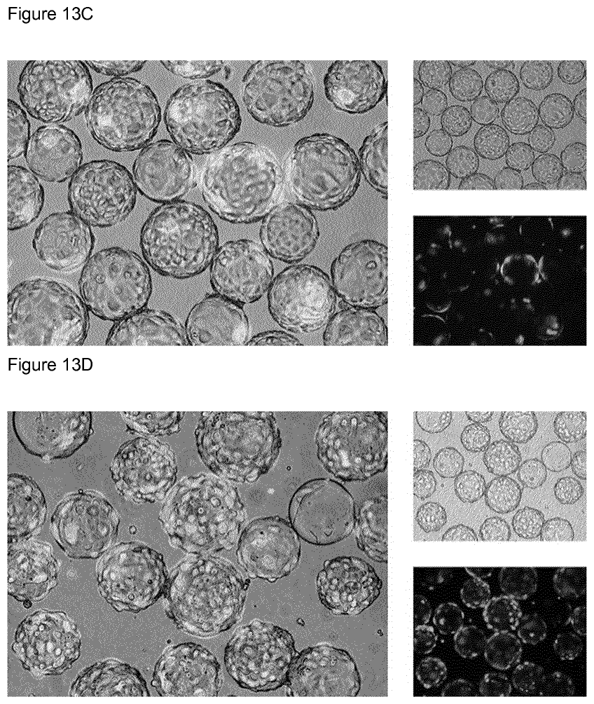

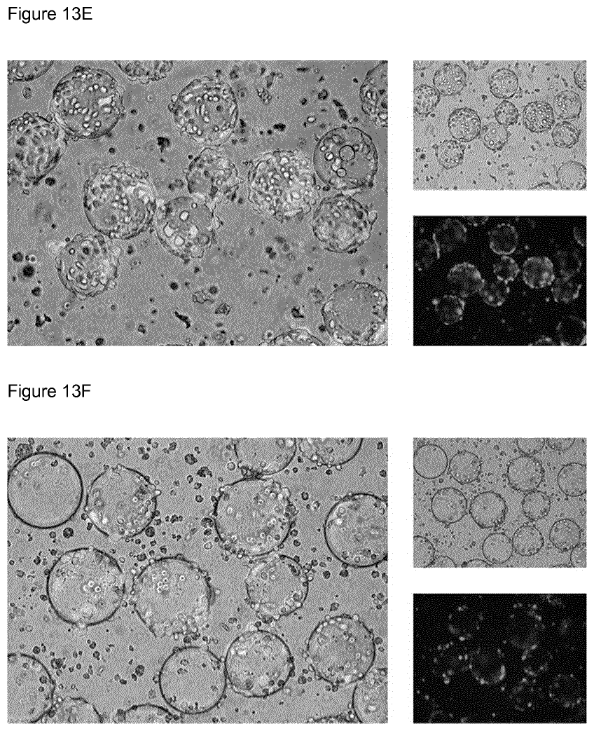

Cell culture supernatant was obtained in an analogous manner as described for MV above in the example section with the difference that the host cells were infected by MV-GFP encoded by a nucleic acid sequence according to SEQ ID NO:4. Progress of infection and virus production was monitored by fluorescence microscopy (FIG. 13 A to F).

The filtered bulk harvest material (i.e. clarified cell culture supernatant) was then treated for 1 h with Benzonase purity grade II (Merck KgA, Darmstadt Germany) at a final concentration of 50 U/mL in the presence of 2 mM MgCl₂ at room temperature and 50 rpm. Digestion was stopped by adding 5 mM EDTA. with an endonuclease and filtered.

The Benzonase treated cell culture supernatant was then directly loaded onto a Capto Core 700 column and chromatographic purification carried out using a procedure that was substantially the same as exemplified above for MV. More specifically, chromatographic experiments were performed on an ÄKTA explorer 100 equipped with a P-960 sample-pump and fraction collector (Frac-950) (GE Healthcare, Uppsala, Sweden). Unicorn software 10.1 was used for control and data acquisition. Conductivity, pH, and absorbance at 280 and 260 nm were monitored simultaneously. Preparative purifications of MVs was conducted by flow-through chromatography using a Capto Core 700 packed in XK 16/20 column (GE Healthcare, Uppsala, Sweden) with a column volume of 9.25 mL. The column was packed according to the medium manufacturer's instruction. Bulk harvest material, was filtered and endonuclease treated and directly loaded on the column. Equilibration and wash was performed for 10 CV with 50 mM HEPES, 150 mM NaCl pH 7.5. The flow velocity during purification was adjusted to obtain a constant residence time of about 4 min. The purified material was collected during the flow-through fraction and was stored at −80° C.

For electrophoresis under reducing conditions NuPAGE Bis/Tris gels 4-12% (Invitrogen, Carlsbad, Calif., USA) and MES-SDS running conditions were used in accordance to manufacturer's instructions. If required, samples were diluted in deionized water to obtain similar protein concentrations. SeeBlue® Plus2 Pre-stained Protein Standard (Invitrogen, Carlsbad, Calif., USA) or Page Ruler Plus Pre-stained Protein Ladder (Thermo Fisher Scientific, Waltham, Mass. USA) was used as the protein molecular weight ladder. Protein bands were stained by Coomassie Brilliant Blue G-250 based EZBIue™ Gel Staining Reagent (Sigma Aldrich, St. Louis, Mo., USA) or by silver using a silver staining procedure as described by Heukeshoven et al. [3]. After SDS-PAGE, proteins were transferred onto a 0.2 μm nitrocellulose membrane using a Trans-Blot® Turbo™ Transfer System with Trans-Blot Turbo Mini Nitrocellulose Transfer Packs (Bio-Rad Laboratories, Hercules, Calif., USA). After electro blotting the membrane was blocked with 3% BSA in PBS-T (0.1% w/v Tween-20 in PBS) for 2 h at room temperature. For detection a mouse monoclonal antibody against Measles NP (3E1) (Santa Cruz Biotechnology, Dallas, Tex., USA) with a dilution of 1:1000 (in PBS-T containing 1% BSA) was incubated for 2 h at room temperature. The secondary antibody, anti-mouse IgG conjugated with alkaline phosphatase (Sigma Aldrich, St. Louis, Mo., USA), was diluted 1:1000 in PBS-T with 1% BSA and was incubated for 1 h at room temperature. Visualization of bands carried out by BCIP®/NBT solution (Sigma Aldrich, St. Louis, Mo., USA), according to manufacturer's instructions. Wash steps between individual steps were performed three times with 1% BSA in PBS-T for 10 min each.

The total protein concentration was determined by Bradford assay using Coomassie blue G-250-based protein dye reagent (Bio-Rad Laboratories, Hercules, Calif., USA). The calibration curve was obtained by bovine serum albumin (BSA) standards diluted in TE-Buffer. DNA content was determined by Quant-iT™ PicoGreen® dsDNA kit (Life technologies, Waltham, Mass., USA). Protein and DNA assays were performed according to the particular manufacturer's instructions in 96-well plate format. Signals were measured by Infinite F220/M200 PRO plate reader (Tecan, Männedorf, Switzerland).

For the determination of a virus sample two 96 well plates were seeded with 5000 Vero cells/well and incubated at 37° C. for 1-2 hours. In the meantime, virus dilutions were performed. Therefore, a 96 well plate containing 200 μL VP-SFM medium supplemented with antibiotics each well was prepared. 50 μL virus sample was applied to row 1, mixed thoroughly and serially diluted to row 11. Row 12 served as a negative control. The cells were infected by addition of 20 μL virus dilution/well and incubated at 37° C. over night. 100 μL supplemented VP-SFM medium was added to each well and then incubated for another 6 days. Each well was observed under a microscope for cytotoxicity or syncytia formation, if one was present the well was noted positive. With the numbers of positive and negative wells per plate a TCID 50 titer was calculated using the Spearman-Kärber formula.

Process performance is summarized in Table 20. Here, the results obtained for MV-GFP (purification run 5) is compared to the results obtained for MV-CHIK (purification runs 1 to 4).

TABLE 20
Summary of MVs purification by flow-through chromatography in different runs
using starting material batch A, B, C and D.

| | | Purification run | | | | |
|---|---|---|---|---|---|---|
| | | 1 | 2 | 3 | 4 | 5 |
| | | BH batch | | | | |
| | | A | B | B | C | D-GFP |
| Chromatographic | Column | XK 16/20 | XK 16/20 | XK 16/20 | XK 50/30 | XK 16/20 |
| parameter & | Column volume (mL) | 10.25 | 9.25 | 10.46 | 188.50 | 9.25 |
| column characteristic | Loading volume (CV) | 48 | 56 | 51 | 48 | 55 |
| | Flow velocity (cm/h) | 75 | 69 | 78 | 144 | 69 |
| | Residence time (min) | 4.1 | 4 | 4 | 4 | 4 |
| Product | BH titer (log₁₀ TCID50/mL) | 5.4 | 5.3 | 5.1 | 5.1 | 6.2 |
| | Load titer (log₁₀ TCID50/mL) | 5.4 | 5.1 | 5.2 | 5.5 | 7.0 |
| | Final titer (log₁₀ TCID50/mL) | 5.3 | 4.8 | 5.2 | 5.4 | 6.1 |
| | Recovery (%) | 73.7 | 36.6 | >99.9 | >99.9 | 83.6 |
| Putity | BH total protein (μg/1 × 10⁵ part.) | 93 | 158 | 289 | 185 | 8 |
| | Load total protein (μg/1 × 10⁵ part.) | 86 | 224 | 174 | 75 | 2 |
| | Final total protein (μg/1 × 10⁵ part.) | 20 | 66 | 37 | 7 | 2 |
| | Overall total protein depletion (%) | 84.0 | 84.8 | 82.5 | 92.3 | 77.9 |
| | BH dsDNA (ng/1 × 10⁵ part.) | 96 | 71 | 149 | 261 | na* |
| | Load dsDNA (ng/1 × 10⁵ part.) | 29 | 55 | 40 | 72 | na* |
| | Final dsDNA (ng/1 × 10⁵ part.) | 26 | 55 | 29 | 50 | na* |
| | Overall dsDNA depletion (%) | 80.1 | 72.0 | 73.2 | 57.9 | na* |

*The dsDNA content of a MV-GFP cannot be measured by Picogreen-Assay (fluorescent assay).

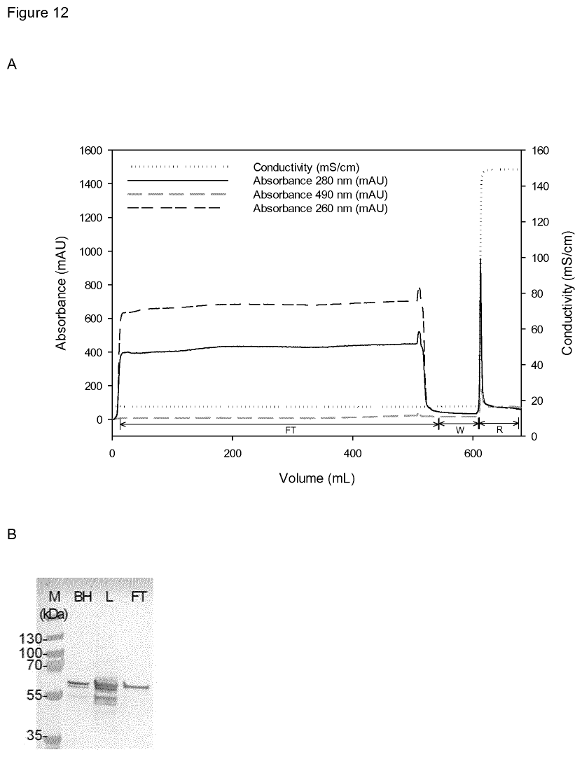

The chromatogram and the Western blot analysis and mass balance of the purification is shown in FIGS. 12A and B, respectively, and Table 21.

TABLE 21
Mass balance of MVs purification by flow-through chromatography in 10
mL scale using a XK 16/20 column. The loading material was filtered and
endonuclease treated cell culture supernatant containing MV-GFP.

| | | Viral titer | | Total | |
|---|---|---|---|---|---|
| | Volume | (log₁₀ | | protein | |
| | (mL) | TCID50/mL) | % | (μg/mL) | % |
| BH | 502.7 | 6.2 | 100.0 | 148 | 100.0 |
| Load | 508.8 | 7.0 | 553.8 | 150 | 102.2 |
| FT | 535.3 | 6.1 | 83.6 | 31 | 22.1 |
| W | 66.3 | 5.2 | 1.2 | 27 | 2.4 |
| R | 92.5 | 4.4 | 0.2 | 31 | 3.8 |
| Mass balance | | | 85.0 | | 28.2 |
| chromatography | | | | | |

| | Total protein/10⁵ | | | GFP/10⁵ |
|---|---|---|---|---|
| | inf. particles | GFP | | inf. particles |
| | (μg/1 × 10⁵ part.) | (μg/mL) | % | (μg/×10⁵ part.) |
| BH | 8 | 51.3 | 100.0 | 2.9 |
| Load | 2 | 57.1 | 112.6 | 0.6 |
| FT | 2 | 2.0 | 4.2 | 0.1 |
| W | 16 | 0.9 | 0.2 | 0.6 |
| R | 130 | 0.9 | 0.3 | 3.8 |
| Mass balance | | | 4.7 | |
| chromatography | | | | |

n.d.: not detectable

The results demonstrated that the GFP tightly bounds to the column. The GFP sticks to the column which was visible by eye, due to the yellow color of GFP. The MV-containing flow-through can then be subjected to (ultra)filtration and/or buffer exchange es described above exemplarily for MV-CHIK.

### Example 22: Immunization Experiments

To evaluate the immunogenicity of the purified material, i.e. infectious virus particles derived from a MV scaffold (MV-Xp), in comparison to the crude, unpurified material (MV-Xup) two animal studies can be conducted:

1. Challenge study—lethal challenge after two immunizations

2. T cell response after one immunization

The animal model of choice would be a transgenic mouse carrying the human MV entry receptor CD46. In addition, these mice are deficient in the type 1 interferon receptor (CD46^{tg}/IFNAR^{-/-}). In previous studies the immunogenicity of various MV/Schwarz based construct was demonstrated (MV-CHIK, MV-DVAX1, etc.). For MV-CHIK we showed that doses as low as 1×10³TCID₅₀ fully protect animals against a lethal dose of CHIKV. Thus, a lower dose would allow the comparison between two formulations in terms of potency. A result of this type of study would be:

Formulation A (purified, MV-Xp) protects x out of 10 mice

Formulation B (unpurified, MV-Xup) protects y out of 10 mice

For the challenge study we propose the following study set up:

CD46^{t9}/IFNAR^{-/-} mice will receive two immunizations. The lethal challenge with the respective pathogen will show % protection against death. In addition, antibody levels as determined by ELISA can be quantified and compared.

TABLE 22

| | No of | | Dose | Vaccination | |
|---|---|---|---|---|---|
| Group | Mice | Treatment | (MV-X) | Schedule | Challenge |
| 1 | 10 | MV-Xp | 1 × 10² | Day 0, 28 | Day 56 |
| | | Formulation | | | |
| | | A | | | |
| 2 | 10 | MV-Xup | 1 × 10² | Day 0, 28 | Day 56 |
| | | Formulation | | | |
| | | B | | | |
| 4 | 5 | MV-Schw | — | Day 0, 28 | Day 56 |

T cell study—IFNγ producing cells after one immunization

Mice will be immunized with a low dose of MV-X (Formulation A (purified) or B (unpurified)) or a control MV/Schwarz. One week after immunization the mice will be sacrificed and spleenocytes will be harvested. The cells will be challenged in vitro with pathogen specific peptides and the number of interferon gamma (IFNγ) producing T cells will be determined by ELISPOT.

TABLE 23

| | No of | | | Vaccination | Spleenocyte |
|---|---|---|---|---|---|
| Group | Mice | Treatment | Dose | Schedule | harvest |
| 1 | 5 | Purified | 1 × 10³ | Day 0 | Day 7 |
| | | MV-Xp | | | |
| 2 | 5 | Unpurified | 1 × 10³ | Day 0 | Day 7 |
| | | MV-Xup | | | |
| 4 | 5 | MV/Schw | — | Day 0 | Day 7 |

### Example 23: Toxicity Studies in Macaques

To evaluate the safety and potential toxicity of the immunogenic and vaccine compositions as produced according to the present invention, the following experiment can be performed under good laboratory practice (GLP) conditions as pre-experiment potentially followed by Phase 1 clinical trials. One group of five male and five female purpose-bred cynomolgus macaques is treated on days 1, 22 and 36 by intramuscular route of the test immunogenic or vaccine composition. The animals are sero-negative to measles. Furthermore, animals have to be sero-negative for the antigen comprised by the MV scaffold and presented in the recombinant infectious virus particles. For MV-CHIK (e.g., SEQ ID NO:5) obtained by the method according to the present invention, treatment is performed at a dose of 1.925×10⁶ TCID₅₀/day of injection. Two other groups of two males and two females receive the composition at doses of 1.925×10⁴ or 1.925×10⁵ TCID₅₀/day of injection. A further control group of three males and three females receives vehicle only (sterile saline). A summary of treatment groups is presented in Table 24 below. The person skilled in the art will readily be able to adapt said scheme to any recombinant infectious virus particle as immunogenic and vaccine composition purified according to the methods of the present invention.

TABLE 24
Summary of treatment groups for cynomolgus macaque toxicity studies

| | Males (M)/Females | Dose (TCID₅₀/day | Volume |
|---|---|---|---|
| | (F) | of injection) | administered (mL) |
| Group 1 | 3 M/3 F | 0 | 2.5 |
| Group 2 | 2 M/2 F | 1.925 × 10⁴ | 0.025 |
| Group 3 | 2 M/2 F | 1.925 × 10⁵ | 0.25 |
| Group 4 | 5 M/5 F | 1.925 × 10⁵ | 2.5 |

TCID₅₀ = 50% tissue culture infective dose

At the end of the treatment period (day 37), the animals are sacrificed, except for the last two animals of each sex in Group 4, which are observed for a 13-day treatment-free period (and sacrificed on day 50). Blood samples are taken for the determination of serum levels of antibodies against the vaccine antigen, for measles serology and for haematology and biochemistry. Other assessments known to the skilled person can comprise body weight, functional observation battery, rectal temperature, ECG and ophthalmology examinations. On completion of the treatment period or treatment-free period, the animals are sacrificed and a full macroscopic post-mortem examination is performed. Designated organs can be weighed and selected tissue specimens can be preserved. A microscopic examination can be performed on designated tissues from Group 1 and Group 4 animals sacrificed on completion of the treatment period.

The following results are expected. No unscheduled deaths occurred during the study. There were no test item-related clinical signs during the treatment and treatment-free periods. In particular, no local reactions were reported. There were no test item-related findings at functional observation battery. There were no effects on the rectal temperature or body weight throughout the study. Qualitative and quantitative parameters at ECG examination were unaffected throughout the study. No test item-related ophthalmological findings were observed at the end of treatment or the treatment-free period. No remarkable changes were noted in haematological parameters at the end of the treatment period, while slightly increased lymphocyte counts were recorded in males and females at the end of the treatment-free period. After each round of vaccination and then in detail at the end of the treatment period injection site inflammatory lesions (e.g. increases in inflammatory mononuclear and/or granulocytic cell infiltrates or interstitial oedema).

1. A method for producing and/or purifying measles virus (MV) particles from a sample, the method comprising in sequential order the following steps:
(i) loading a sample containing MV particles and one or more impurities onto a stationary phase material for carrying out flow-through chromatography to bind at least a fraction of the one or more impurities contained in the sample and to produce a flow-through comprising at least a fraction of the MV particles contained in the sample; and
(ii) carrying out filtration, and obtaining a retentate having an increased MV titer relative to the MV titer comprised in the flow-through.

2. The method for producing and/or purifying MV particles according to claim 1, wherein the sample is obtained by a method comprising one or more of the following steps:
(a) infecting at least one host cell with a virus stock comprising at least one MV particle;
(b) incubating the at least one host cell infected with the virus stock to allow virus production;
(c) obtaining a cell culture supernatant containing MV particles and one or more impurities; and
(d) clarifying the cell culture supernatant to obtain a clarified cell culture supernatant.

3. The method for producing and/or purifying MV particles according to claim 1,
wherein the sample loaded onto the stationary phase material for carrying out flow-through chromatography has a MV titer less than 5 times higher, less than 4 times higher, less than 3 times higher or less than 2 times higher, than the MV titer in the cell culture supernatant obtained in step (c) or in the clarified cell culture supernatant obtained in step (d), and/or
wherein the sample loaded onto the stationary phase material for carrying out flow-through chromatography has a host cell protein content of at least 50%, at least 60%, at least 70% or at least 80% relative to the host cell protein content in the cell culture supernatant obtained in step (c) or in the clarified cell culture supernatant obtained in step (d).

4. The method for producing and/or purifying MV particles according to claim 1,
wherein the sample containing MV particles is directly loaded onto the stationary phase material for carrying out flow-through chromatography, and/or
wherein no concentration step and/or no buffer exchange step and/or no host cell protein removal step occurs in between.

5. The method for producing and/or purifying MV particles according to claim 1,
wherein the MV titer in the flow-through is at least 20%, at least 30%, at least 40%, at least 50%, at least 70%, at least 80% or at least 90%, of the MV titer in the sample loaded onto the stationary phase material for carrying out flow-through chromatography; and/or
wherein the host cell protein content in the flow-through is 60% or less, 50% or less, 40% or less, 30% or less, 25% or less or 20% or less, relative to the host cell protein content in the sample loaded onto the stationary phase material for carrying out flow-through chromatography; and/or
wherein the polynucleotide content in the flow-through is 70% or less, 60% or less, 50% or less or 60% or less, relative to the polynucleotide content in the sample loaded onto the stationary phase material for carrying out flow-through chromatography.

6. The method for producing and/or purifying MV particles according to claim 1,
wherein the flow-through chromatography step involves column chromatography;
wherein the stationary phase material comprises a resin, a matrix, a gel or beads; and
wherein the stationary phase material has size-exclusion functionality, hydrophobic interaction functionality or ion exchange functionality, or a combination thereof.

7. The method for producing and/or purifying MV particles according to claim 1,
wherein the stationary phase material comprises a ligand-activated core, and an inactive shell comprising pores,
wherein the pores have a molecular weight cut off smaller than the MV particles to exclude the MV particles from entering the ligand-activated core,
wherein a molecule smaller than the molecular weight cut off can enter the pores and bind to the ligand-activated core, and
wherein the molecular weight cut off is from 100 kDa to 2000 kDa, from 200 kDa to 1500 kDa, from 400 kDa to 1200 kDa or from 500 kDa to 1,000 kDa.

8. The method for producing and/or purifying MV particles according to claim 1,
wherein the flow-through is directly used as a feed for the filtration.

9. The method for producing and/or purifying MV particles according to claim 1,
wherein simultaneously with or sequentially to the filtration the retentate buffer is exchanged.

10. The method for producing and/or purifying MV particles according to claim 1,
wherein the filtration involves tangential flow filtration.

11. The method for producing and/or purifying MV particles according to claim 1,
wherein the MV particles are selected from the group consisting of live, attenuated and inactivated virus particles, or a mixture thereof; and/or
wherein the MV particles are recombinant and/or infectious particles.

12. A system for producing and/or purifying MV particles, the system comprising:
(i) at least one bioreactor;
(ii) a clarification unit downstream to the bioreactor;
(iii) a flow-through chromatography unit downstream to the clarification unit; and
(iv) a filtration unit downstream to the flow-through chromatography unit.

13. The system for producing and/or purifying MV particles according to claim 12,
wherein the at least one bioreactor includes a probe for determining viable cell density based on permittivity,
wherein the probe preferably allows for online determination of the viable cell density.

14. The system for producing and/or purifying MV particles according to claim 12,
wherein no concentration unit is arranged between the at least one bioreactor and the flow-through chromatography unit.

15. The system for producing and/or purifying MV particles according to claim 12,
wherein the clarification unit is either in direct fluid communication with the bioreactor or via a first vessel; and/or
wherein the flow-through chromatography unit is either in direct fluid communication with the clarification unit or via a second vessel; and/or
wherein the filtration unit is either in direct fluid communication with the flow-through chromatography unit, or via a third vessel.

16. The method for producing and/or purifying MV particles according to claim 1, wherein the filtration is ultrafiltration.

17. The method for producing and/or purifying MV particles according to claim 7, wherein the ligand-activated core comprises octylamine.

18. The method for producing and/or purifying MV particles according to claim 10, wherein the tangential flow filtration is carried out and maintained throughout the filtration at a transmembrane pressure of from 0.1 to 1 bar, from 0.2 to 0.8 bar, from 0.3 to 0.6 bar or from 0.4 to 0.5 bar.

19. The system for producing and/or purifying MV particles according to claim 12, wherein the clarification unit is a dead end filter unit.

20. The system for producing and/or purifying MV particles according to claim 12, wherein the filtration unit is a tangential flow filtration unit.


SEQUENCE LISTING (7 sequences)

SEQ ID NO: 1 - DNA, 18994 bp, Artificial Sequence
  MV Schwarz pTM
tcatgaacgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtgatccttataaagctagatg

SEQ ID NO: 2 - DNA, 19075 bp, Artificial Sequence
  pTM 2ATU MV Schwarz short
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 3 - DNA, 19107 bp, Artificial Sequence
  pTM 2ATU MV Schwarz long
tccaaccgcgcgcgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtgatccttataaagctagatg

SEQ ID NO: 4 - DNA, 19822 bp, Artificial Sequence
  MV GFP
tcatgaacgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtgatccttataaagctagatg

SEQ ID NO: 5 - DNA, 22857 bp, Artificial Sequence
  pTM 2ATU MV CHIK
tccaaccgcgcgcgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggagttcatcccaacccaaactttttacaataggaggtaccagcctcgaccctggactccgcgccctactatccaagtcatcaggcccagaccgcgccctcagaggcaagctgggcaacttgcccagctgatctcagcagttaataaactgacaatgagagcggtaccccaacagaagccacgcaggaatcggaagaataagaagcaaaagcaaaaacaacaggcgccacaaaacaacacaaatcaaaagaagcagccacctaaaaagaaaccggctcaaaagaaaaagaagccgggccgcagagagaggatgtgcatgaaaatcgaaaatgattgtattttcgaagtcaagcacgaaggtaaggtaacaggttacgcgtgcctggtgggggacaaagtaatgaaaccagcacacgtcaagggcaccatcgataacgcggacctggccaaactggcctttaagcggtcatctaagtatgaccttgaatgcgctcagatacccgtgcacatgaagtccgacgcttcgaagttcacccatgagaaaccggaggggtactacaactggcaccacggagcagtacagtactcaggaggccggttcaccatccctacaggtgctggcaaaccaggggacagcggcagaccgatcttcgacaacaagggacgcgtggtggccatagtcttaggaggagctaatgaaggagcccgtacagccctctcggtggtgacctggaataaagacattgtcactaaaatcacccccgagggggccgaagagtggagtcttgccatcccagttatgtgcctgttggcaaacaccacgttcccatgctcccagccaccttgcacgccctgctgctacgagaaggaaccggaggaaaccctacgcatgcttgaggacaacgtcatgagacctgggtactatcagctgctacaagcatccttaacatgttctccccaccgccagcgacgcagcaccaaggacaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtcctaaaggcgaaactctgacggtgggattcactgacagtaggaagattagtcactcatgtacgcacccatttcaccacgaccctcctgtgataggtcgggagaaattccattcccgaccgcagcacggtaaagagctaccttgcagcacctacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccggaaaggaaaaattcacatcccgtttccgctggcaaatgtaacatgcagggtgcctaaagcaaggaaccccaccgtgacgtatggcaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcacgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtcccattcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcttacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccttacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgcccttcggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaataggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtgatccttataaagctagatg

SEQ ID NO: 6 - DNA, 20661 bp, Artificial Sequence
  pTM 2ATU MV DVAX1 long
tccaaccgcgcgcgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatgggcatcatattcattcttcttatgctggttacaccgtctatggcggagaaacttcgaatcaaaggtatgagctatacgatgtgcagcggcaagttcaagatcgagaaggaaatggctgaaacccagcacggtacaactgtggtcaaagtcaaatatgagggggctggcgctccctgtaaagtacccattgagattagggacgtcaataaagagaaggtggtaggtcgcatcatctccagtacacctttggccgagaacacgaactccgtcacaaacatagagttggaacccccgttcggagactcatacattgtgatcggggtgggcaactctgcactcacactgcattggttcaagaagggaagcagtatcggccgcagggataagagagacaaactgaaattgaaaggtatgtcctatgccatgtgcacgaatactttcgttctcaagaaagaagtatctgagactcagcacggaaccatcctgatcaaagtcgagtacaaaggagaagacgtgccctgtaagatcccattcagtaccgaggatggacagggcaaggcccataacggcaggctgataaccgccaaccctgtggttacaaagaaggaagagccagtcaatatcgaagctgagccaccgttcggggagagcaacatagtaattggcataggggataatgctttgaagatcaactggtacaagaaaggaagctccattggccgaagagataagcgcgacaaactccagctgaaaggaatgagctactccatgtgtactgggaagttcaagattgtcaaggaaatcgccgaaactcagcatggcactattgtgatccgcgtgcagtatgaaggcgatggtagcccctgcaagataccatttgaaatcaccgatttggagaaacggcacgtcctgggtcggctcattaccgtgaacccaatcgtgaccgagaaggacagtccagttaatatcgaggccgagcctcctttcggcgacagttacatcattgtaggggtggaaccagggcaactgaagctgaactggttcaagaaaggcagcagtataggacggcgggataaacgggacaaactcacactgaaaggcatgtcatacgttatgtgcaccggctcattcaaactggagaaggaagttgcagagacacagcatgggaccgtgctcgtgcaggtcaaatacgagggcaccgacgctccttgcaagattccgttcagtacacaggacgagaaaggcgtgactcagaacggcagattgattacagcgaaccctatcgtgactgacaaggagaagccagttaacatcgagactgagccgcctttcggagaatcatacattatcgtgggagccggcgagaaggcactgaaactcagctggttcaagaagggcagctcaatcggtcggagagacaagcggtctgtcgccctcgcaccgcacgtgggcctgggtctggaaacgaggaccgagacgtggatgagttccgaaggcgcatggaagcaaatccagaaagtggagacgtgggccctcaggcatccgtaatgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtgatccttataaagctagatg

SEQ ID NO: 7 - DNA, 20896 bp, Artificial Sequence
  MV Zika sE
tcatgaacgcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccaccatggcacgcagaggagcggatacgtcagttggcattgtgggcttgctcctcaccaccgctatggccgcagaagttacaagaagagggtctgcctactacatgtatctggacaggaacgatgctggcgaagccatctcctttcccacaaccctggggatgaacaagtgctacattcagatcatggatcttggtcacatgtgcgatgccacaatgtcctatgagtgtcccatgctcgatgagggcgtcgagcctgacgacgttgattgctggtgtaacaccaccagcacctgggtggtctatggcacttgccatcacaagaagggtgaagcacggaggtcacgacgggcagtgacattgccaagtcatagcactaggaaactgcagactcgctcccagacctggcttgaatcccgagagtacacgaaacacctcattcgtgtggagaactggatcttccggaatcccgggtttgccctggctgctgccgctattgcctggttgctcggctctagcacatcacagaaagtcatctacctggtgatgatcttgctcattgctccagcctactctatcagatgcatcggagtttccaatcgtgactttgtcgaagggatgtccggaggcacctgggttgacgtggtgcttgagcatggtggctgcgtaacggtaatggctcaggacaaacccacggttgacatagaactggtgaccacgactgtcagtaatatggccgaagtgcgaagctactgctatgaggcctctatctctgacatggcatcagacagccggtgtccaactcaaggcgaagcttatctggacaagcaatccgatacacagtacgtgtgcaagcggactctcgtggatagaggttggggcaatgggtgtggtctgtttgggaaaggcagtctggtaacatgcgcaaagttcgcatgttccaagaaaatgacaggaaagagcattcagccggaaaatctggagtataggattatgctcagcgtgcatgggagccagcacagtggcatgattgtcaatgatacaggccacgaaaccgacgagaacagagcgaaggttgagatcactcccaattcacctcgcgcagaggccactctgggagggtttggaagtctcggtctggactgtgaacctcgcactggcctggacttcagcgatctgtattacctgacgatgaacaacaagcattggctggtccataaggagtggttccatgatattccgcttccttggcatgccggagccgataccggaacaccccactggaacaacaaagaggctttggtggagttcaaagatgcacacgctaagcgccaaaccgtcgtggtcctgggtagccaagaaggagcggttcacactgcgcttgccggagccctggaggcggaaatggatggggccaaagggcgcctttccagtggacacctgaagtgtcggctgaaaatggacaagctgagactgaaaggtgtgtcttacagcctgtgcaccgcagccttcactttcaccaaaatccctgctgaaaccttgcacggaacagtgacagtagaggtgcagtatgcaggcacagatggcccatgtaaggtgccagctcagatggcagtggacatgcagaccctgactccagtgggaaggctcataactgccaatccggtcatcacagagtctaccgagaactcaaagatgatgctggagctggaccctccctttggggactcttacatagtcataggggtaggcgagaagaaaatcacccatcactggcacaggagctaatgataagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtgatccttataaagctagatg